SEQUENCE LISTING (109 sequences)

SEQ ID NO: 1 - DNA, 62037 bp, Unidentified
  misc_feature 1..62037: note = Bacteriophage strain LSP1
  source 1..62037: mol_type = other DNA
ttattccaccctttcttctttatatagtgcgtcatattttgctgttccatcatagcgcagcgtcccatcataataagctacattgcgctgtgttatgatctgcatatttcctagttgtccacagggactatttatattcatttgtatcttaattttatctactactctaggtctgttgtttttaaccttgcctttatatctatgccaaatatttaaaatatttttatgtagcacctgcatagacaaaatctggctgttatggtttatttcttctacaacattcaattttattaaatgctgcactatcgatttcaaaataaattccttctgccttattctgtaaggaaaatacagtaacaaatctgtctgcattctgttctgtacaagcactcttgtacagttcctactgcatacaatcctttgtaatgttaaaatgcctatataatttcttactataatacagaccttatagtttcttacagcgttatacagaagtgtgccatcgtagcacgcctgaccattgtatacactacagccccaaaaacttatactgtaaaagctgcatagggacagatttgtatattctctcccttgtatgaatatcttaattctgttatacttgtatgctacatgctgttgtgtctgtgtatgcatcaaatatcttattaacagcccaattttatattttcttacagcattatatagaagtgtgccatcatagcagtatggtccagtataaatatcatctccccagaatggaatatacaaaataaatatacaggcaaccctaagctgctctaaatgtacattattaaagccaatctcaattctgtcatttatgatatagattgtatgcgactgctttaacttgtttaatacatcatgcaccaactttgaatttaatgttccctcccccaaaaagtaagccttaaaaacatttgggtgcgaaggaacaaatccatacactcctaaatcatttgcatctgcaatatgtacttcaaaattcgtaacattttccagatatttttccattcggtaaggggtcatgggcgctctgcaatcccttttttgataaattaagctgcgccgttcttcatctgacaaattcaatcgtactggcaatccccacttaatttcatggtacatcagtccccatgtagctgtttctggaaaaaactgtacaggcagctcctcaataatatccaacactttatcatattctagccccattacttgatacagccatttgccaatataagatttatcatagaaaccatctgacacataacttaacatcttttgggcactaccgcttgtaggaaaatgctccaagtcaaatttctcttttttcatcaagctgcccccttaactgaattgaaaattgccagttttcgggtattcttctttattcaattgaatattctctctttcaccattcataagaaattcatcaaaatctattacaccagcaatattagagattatcggtcttacatcattataccgcagtatattttgttgttttgcaactgtatataccgctttcaccgctgttgaaaagtccttctcaatctgctttattgtggtggtttcatcatagaaaagccctgtaattacataatctatctttacggtagtggcaggtccacagattaacttcgcacaggcagtaggcagcagccttttatttctatcttctggggatacaatataattgtatactttatgtacaagctcatcattggctggctgaccattagcatctaccaaaacaatttttactgttcctggtccctctgctgttggaatcacaatacagtcaccagcaccagactcctttgcccaccgaatataatcactatcattccctaaaaaagttaaactgttatcatattccgtcgcaattctatcatagaaattgtcgttgctctccctttcggttcctcctgttatctgttctggattaattacctctgtgatgtttttatctggttttgccataagagttacagtatttgctggcacattagagctggcaccactttccaccgctaatacaggaataagaattgtcccttccgcctcaatttctttttcttctgtagaataaaacagaatagacggtccactatctgttgctggtgtgcaaaagattgttccctctgctataattgttcctggtatgccagtaactttcacttttcctgatgcacactttggcgggtgcctttcaagatgcacctgctgaccgtgaagatctagccagttatcccatgcatattgcggaaaagcaatcattaaagcccttactagatggtaattaataaattcgtctttctctagtgcagcaggtcttgtaaaatcatagggaaagcctcctggcatattatcaatgtctgctggaaggttgttcatcattcgctgatgaatttcatctgcactgttattttcaataaaatctggtcttataaattccggctgcatatcctctccccctcttaaatcgtaattgtgatttccttatcacaatcaacaccctttactttaaatttacaatgcatactgtcaccttcccaggaaaattcaaaatccagaacgtcctctgtcctaggatttaccataagcgcttctgtaatggttctctgcaccatagattccactgtttctacatcgtcattatccaaagcacgttccatttcaacgccaatagagcctggatatgcaagacaacggtaccgctctgtttgtgcaatcttaaaacaccatatagcgtaagcctcttttccatcacattctacaatacggttcgcaccatctctaacaaaatcattggttacagggtcccattttgcacttcgcctgtattccatatcatatcttgtattttgtggtataaattctggtacttgcaccactgggaatagtggttgtgacatctgcctcgcctccttcttatgattgctcaattacatctattaccaccgcttcactttgcacccatgccactaaaacacgatctcctgcttttaattctggcagcagtacatcatggctgtgtccccaaaaccccgaaggattatcatgtatatgcttatccccctgtatatgccctatccagctagactctgatgtccctagcggatacccattaatagatctgcaaacagagtaatcaccctttggtattggaactggaaaagtatttgttacaagactataatttccctgtatctctccaaaatctaaaacaaaaggtgattctgtttcttttctcattctttcgctcaagacagttgctagttttgctgtgccagggtgtccgtcaaatccttccatctataccctcctctaatcaaatgttccatcatctgcttccccagcgctgctgttgcgacgtcgcaacagagtccctcctctaatcaaatgttccatcatctacccacccatatacattactgccactatctgtatggattagatgccatgggtgcgctttgcctgaaccatttttaatagtgatttttgcctttccagcgcttgcaggataccctttcgcatcagaataactactgacatagtgtgtacctccatggaagttcacaatatctcctacctgatatgctttcttctcctgtccattatcactatccccacttatttcttctgattcaacgtactccaaatccatagacatgttataagactctgcattatgctgaatacttttaacataataataacttgatgttatactgcttataatgtagactagatcaccttttcgtataaatggaacatctggtgcttgtactgccattttcttttcaatcttgccctcgtcctctagtatcttttgtgccgccagatttgcatcacttaatgtttcctcttttcctctggtataaatcctttgccgaatcccatactgaaccgctccattcagtgtagcttctacactgtgtttcccttgcttatctgcctgaccaactactttcactctagtaattaaattttctgtgctaatatttttgctaaaggactgtgtattctctacacaaaacacatatacagttttattgctgccccacggaattacactggtatagcctttcgctgcctgtatcatgcactttttctcgccttttttaactgcatcatctaaaaattccagtataatatctgacagatacttgttattacacactgttttcccatgagaggcattgggtcccttataatctccctgcaatatctcccaatcattaaaaatctcctcaattgcggattttgttccagttccagaaggatagtatcggttatcctggcttttctgcaatttatacaattcatcatagcaggtacatttcaagctgttctcactattcttttccacctggttccagttttctacatatcctctggcaacttcctcactgacaacaccatctgtagcaaaaattcctataaggcagccaggcttaataagactggacaaatatccctttgctgtctcataattgcgtaccgtaaagaacaaacgcatagaaatttcgttttcattttcttcccagcctaaattctgaataaagtcaccaatgttatactgatttccagatgcatccattactacagcataatactttatattcgctaaatcaatcatcggtgcctcctatcctggtatagttagcacttctccaggccatatccaatgcccatgatcagaactggacttcttatgtttctttgcctctgcctcaattgtgtcagcatttgcatcatagataacagtccatttgctaccacttcccaatttctttgctgcaatcccccataaggtgtcgccgcttaccactgtataggtacttcctcctttagaagaatcgccagaatcatttcttggcgctgtttttacaattccaagttcttctgttgtataaatttctaaaggctttttttgcacaaaagttatagaatattcaacgtttccgtaagcacctattggccgcgcttgaaaagaggaaatggtaacgtccatattaatccatgtacctgtcacaatcagattcaatactgtttcatttttaataaaattacttagtatttttacacattcttttggttccttccaactgttttgtcttacaatcggttccatctttttagattttccaaagaaaacaccgtcccatgaaaattccgatattgatgttcctttcggaattttcactgtgccttgagagataatgtcaaaactctggtattttgcaccatattttccttgtattttttctggcaatgccggaaacgtaaactttgcaccgcttccaacaggaatcaattttatgtccatagtctacgcctcctttaatggcatattagaaaaaacatcttccaatgttgcagcaatctctccgtttaattcatctgctaattctttcatatgccttctaataatctgcataatattttcttcgctctgtccatcgcccccatgaataacaaactctggtgccaggcttacatttacctgtataggagaactgcttaaactgctttgtctttctgctgatacaggttcatacacctgtacagtttcttcattatagttgtcctgtgtggtttcgttataggctgtagaagcgtatttgtttgcatcattgaataaattatagctacttgctgtattatgagaaatagagcctcctacataaccaccactggcatgtgcagacactcccaatgcgtcacccgcctgcttataaagttctaatgctctggatcgcctgctcggatttgtaggaataacaaactcaccatatccttcctctgccagccatgaaagctgtggaccaccgcttacataaccgcctgccgcatgaccgtccgtctcttggttaaccgttggaagtgtaggtgtggtaatattataagttggtgtcatatttatagatggattaataaaaaacggatccgctatagctgcctctaatgctgtctgcatatctgttcttaaaagaggtaaagaatctaataaagttgctgaagcaccatgactaagcgctgtgccaaaatcaactcctgtatcttcccattcctttattaattcatcataacgctcatttgagactggaccatactgcgccataatatctttaatatttctagcatcatctgaagctgaaaaattaggcatatactgttttgtaaaagcggatagattttccacacttttattgtcaactaaagaattactcatttgttctgcataatcgtctattaactcatcgtaatcttttccgaactgtcttgcccattcctcaagaggcatatccacttgttttataccattatctggtatcacaacgccaacttgttgtagcatattacttacatcattattggttaaagaagaccaatctacagcgtctctaatttcttctgctgttggtacctgttttaaatagttggcgataagttgcctttttgtttcttctggtactgctaacgctgtctgttttaaatccaatgcaattgtagcctgatctgctgaatcaatattaagcttatccagtccaatccacttaataatgtctgtgtctgtccaagctgttatatttgggtgttctataattgtattatttagaacctcacttaatttttcggacaaagaaccttccatttctggcataaaatctgaaagctgttcctcaaacgcagtagcaatactatcaagattaaaggttcgtacatttacatctagttcctctatatttgcctgatatcccgccattgcttgttcatatttatcttgatactctgcatctgtaattgtgccatatgtatactgcaaattaagattggtgattgtttcaactaatgcattgtaatactgtttttccccagctgacttctgttcctgtgcttccacttgaactgcactaaaactatctgcatcaagagcagctcccttatgcttaatctgcaaagcctgaaactctgcatcagtctttgcctttgctatttttcctgtaatttctgcaatctggctctgtaactcctctaatgcctttgcctcatctgggataataatcccatcttctaatgcagcattcattgtatctgtcagttctgcacttaaatcatttacccgattttttactccctcataatagctatccagactggaagtatctgcttcctctcctgtcagcagtttaagcgcaacagtcgcctgataatgattatcgtcaatgaaggtctgacttgcattaaggaaattttcaattgcacctttataactgtccttatctgtttctgataattccatattaagaccgaccttccaattctcctttttcagatcggaaacagacaattccaggctgtttagcgctgcttctgcgtcagatgttacctgtgcaaacatatttaatccctctgccatattaccaaaagtgatttcacttgcaactttctttacctctgccaaagacagggaaatgtcaccaaatgctttcttggcaacattcgcacattcctcctgaaacatcaaagcaaactgacttgcagatacttcagaatcattcatagcctgaaccagcgctttatttttaaatgtcacatcttcaattgcaaatccagtagcctcaaagactttttgtgccttttgcgcttccttctgcatttcttctacatttttctggtattcttcttggtattcttcctttatcttatttcccttaatccaacctgtaatacccccaacaccagcaccaactaaagcacctaccgctgtaccgattccgggaatgaaggaacctatcgctgctcctgtaactgctccggtaacaactccaccggctttccatgctgctgactcgccgtaagcttttgactcttccttattgtcggacttaattgctttgtatgcgtccagggcactacttatcaacgttacacctgcggcgattgccccgccgccagctgccatgcccgttgcgctcaaagcacctgcgctcatagatgcgcctcctgccagatttccggctcctaggttgattgccagcaatccagattttcccaacagtccggtaccagcagccgccgaacccaggaacgatcccatgagggacgtaccagttgccgcatttgcaccaaacaatccttttccaatacttacgcctccttttgccataccaataaacagtttggcaactttactaagcacaatcgctgataaaacagaggacaaccctgctgactcgcctcctggcaatagctttcctgcactagaaaacatgcctttaaatccctgccacagtttacctgatactgtatcaaagtcaaaaccctctgaaaaaccttttgcaaatgcagcaccaaaacttactccttcgtccgaaatctcgctaatattcattcctaacaatgtcataattccagttcttagtccagttccaattccattgccaatattttgtgcaaatttttctttccctgtgccgttccaccattctaagaacggctctgcaataaattcgtcccaggcaatttttacttttccgaagaaatttgcatctttccatttttttgtttttgtaagtttatcaaactttctctgcatttggtctactttcgtatcaacccaattcataagttcatcaagcgcactttctatcataggcatctgttcagttaaccattccgcaaaactccttatataaggagccattctgccaccgaaagaaatttttactccatcagccgcgctctgcaacagggttattgctccttgcagattatccatcataatttcagccattttcgctgcggctccatctgcattatttacagctttggacaatttttcataatcttctgtagttgcgttcaaaattgcaagaaatcctttttgcgcataagttcctgcaacagtattggcaagattcgctttctgctcatctgtaaaatctgctgttgcatctcttaattctttcataatgtccccaaatgctctcgctgaaccatcagaattaaaatacgctatacctaatttttttaaggcatcagtagcaccatgcgtgttggtagacagtctggtaaatatggaatttaatgcagtaccagacatattgcctttaataccagcatttgccattaatccagttgccagcgcaacatcttcaattgaatatttcaaagttccagccatcgctcctgcatacttaaatgtttcacccatcaaagatacattcgtattggaattggacgctgcaactgccataacatcagcaaagtgccctgcttcacttgctttcattccgaaagctgttaaggcatctgtaacaatatctgatgttgtaccaaggctttctcctgatgcggctgctagactaaggattccctcaataccacccatcatatcttctgttttccagcctgccattgccatataattaaatgcttccgcagactccgaggctgtaaatttggtggtagctcccatctcttttgccttatttgtcagttttgtcatttctgaatctgtagcaccgcttatcgcttttacttgggacatagcagcctcaaagcccttataggtatcaattgtatcttttaaaccaatactgactccaaggactgcccctgcttgaaagacgggattctttagcagatttataatccctctaactggtgaagtaattaagtcaaccgctcgcattgtaacattccatgtttttcctgcaaaattttttaacccacttccaatcgtagaaagaattggcgcaattttgtcctttgcttccagcaatatctcatatttttcttttgcccattttgccagacccttttctgttttctgtgcctgcttatcaaactgtgaaacacgttctcctgccttttgagaggaagtgcctgttttttctgccgccgtttccagatttcccaactttcggattaaattcgatatttctggctctgttttatccacagtttcaatgggtatttcaattctaagtgtctctgctattgtcctcaccttctttcgttcgcgaatctaaagttattctcatagatgcaagcataaatgcctgtacgccttttgatttctgataaaattcatctggaggtatgcctgttgtctgaaaaatgtgatggagcaaacaagcctttccaccagccttaattagttttttgccacttcctccagattgtcatcataaccactaagtttatcaatagcctccaggaccttatccttttcaccagcttttagcgaatactcaatcacgtctaagccgttcataatacggtcctctttagcattcagtgcattccaaaccttgtggttatcccacagatttttcctgtcttcctctacagttgcctcgtagataatggcagattgatatttaattctgtctgtatcttctggcagtttcataccaagctgtttattacgtacatatttggtatactttttcttacatttctcgtactcttgagaactaagaggccgaacgtgaaatgcaaaatacagcctgccttctcgaacaatttcgatacgctgggtttcctctgatgcatactctgccgcatcaatcagcccctgaataaaatcttcttcgtttgctcgaattaatattttggtcccttcctcgttggtttcatactctgttatttcttgttcttctactttctgtgttgcctcattggttgtatcgctttctcccatagtcacaccttttgtaaaattgccttttctaaaatcctttgacatctgtatttccccctatttctgtataagaaaaagggcagctctatgccaccctttaattcaatatcctgttatctaatccctatcaattccaagcaatgactgtaatttaggcgctcgattgacaaagaaattccagttacgcttgattacatcacctgttgtaacattctgaatgtccacctgtcctgaagggatacactcacgataaattacacgttcctcagagccatttctgccaagcaatgacccctgaaaatcccatacaggcataacctgcgtttctaatgcttccatgagttcctcaataaacgcatcatcttctaccacaacttgagacatggtaaggctcacagaaaaggtatttgctgtctcatgttcttgtgcgtcgcccaaaacactatacttagcattgttccaactaacatttgatgtaaactgttccactgttgccagcaatacaccatctttgttataaaaagccccatcttttccagttcttccatgtctggaatcaccggctgctctttcatttctcatagtctatatccccctattcttcgttcgtactgaatcggaacaagaatgtcagataaatatgttccatcgaatccttgtcaatcacatcaatatcaaaccatgcactgtctccgtccgccttgtatgcgctgctctccgttaccgcacacgctaccagcttgccttcctcacgcatggaatcaccaacaccctgtacctggctgataacagttgcccttccattgttgtcattgtccaccttgccgaccagatcatctgtggtagtattgatacgccttatcagctcaaaccgtgtctttacccttctaattttcttccagccgtcatcttggttatctgctggcgtgataagtgtattgattgcattatcaatccacacctgctttactttgttgtaacttagtacaatacagccctttttctctgcggaaatcatctctgtatttgtaagcttttcaagaatctccgaaaatccagtaattacagtatgggtaagtgaagaaccggaagacacagcaccaatcatgccagcaatacgtgcggctgtctgatatccgtcaattactgttccctgctcctctatccatgcatttaaaacataattcatcttttcatcattaagtgccgctgcatgtttcattcttgtttctaaatcaacagtatgtttttctgcaacaactgcctgcgcaagagaacccgcatcaaaaattcgattcacaaaagattgtaacagcagatggatttctgtgtcctctgtatcaagacagactgtattaaactcgtatggctcaatctgggcaaaggcattcgcgtaatctcctgtagttgtctctgggtttgtaccactggtaaaggctctttgcgatatcatttccagaacagcgcgctctttattttgctttaactctgctttgaagttcttagagactgccaaggcatctgccagtgcttttgcctcaccttccccagaagcaaattcaaccttctcaaattctgtagtaccagcataaaaaatacattctttcaagctggaatctgacagtttttcccgaactgtaaccataaaattcttttctccagggtatttggctgtgatgcaaagtgtatctgaatcatttacatcttttaaagtaatggttccctgggtgcctccattacctactctgcaagcaataatcgtctttgcaccgcctgcaatcgcctctctcattgcatctgtggttaatccagttccaaaaatcttttcataaccatcttcagcatttaattctactgctgtattcagtggtccgaaatctgccttaaatattacggctgtcactccattcataataccagctacagcattattgccttttttctggatattaaaataggaacctgggcgaacttttgtttctcctaagataaaagtacctgccatttattgaacctcccttttcataaatttttctacaatcccttttgcctccgtaacagtacattctgtttttccagcagcttttaatgctgcctcaatacactcctgtcttacaccaaaaacctttcttgcattcgctgccagctcacttacggaatagatagattcctgtaaagttctctgttcttttttgggtgcctgctcctgcgttacttccactttgatatcagtggtttcgacagttttagtctcctttaatgtttcctttgccattgtatacctcctacttatacctgacatgggctgctgccaaagaatgtggtttttccttataccgcagcagcccataatgacctgttacaaaaatctgaccatcttttaaatagtccgatttataatttgcctgtaaccgtttaatgaacatgggcgaatggtccaacatgataatttctccatctaacgacatacggtttgcaatcacagccgccatcttgagccttgtttcgctctctggacataaaatatggacagcaattctgccgtccagccatgcaactgtatttgtttcttcagacttatctacagataccaggcgacaatatattacgggtcttttccctgttgcctctgtgatttcctccatctggtcatatcccataataaaacactctggatataactctttaatatacttattcgtcgccataattggatctggatcagatgtctcctgcgacggatattccagaatatcaaaccgcacttcacttccaatcgtagtatttcccttcttttcatctatggtgaaagcgtctgtccttgcccatgcaaaacaatatggtgttcctccttctggtttcaatatcacatctcgaaggcagtcctttatgaacggctcaatctgttctggcatgacttctgttgtattctgacagagaagggacacgaaaagcgtgcctgcactgtgccgttcctcatttgccagaaggtcaaagttataaactatctgtggatactgtgtatttccaccccacccttcctgactttcttctggtggctctgtactgaaaatagcaggaccaccaccaaatgatgcaagattttctataaggttctttgaacaaataaaccgcttataaatcagttcttctaatatcattgtcctccactccttttgtctcctgctgctccttatctgtctctacttcatattcctttatctctgtcatatcagcagaccatcgaatctcccattgtccgtcgattacctcggacaccggaataacaaaatgatttgttacattaccaattccaggataatactgtacaattaactgctgttctgctgccgttgtaacaaaaccggcttttccttcattccatgtacaatgtttaccccaaagcaaatctccgcgcttaatttgggacatatcaaaggcggctgttggtttttccattattagtgccatagcttacccccctatgtataaggctcgctgtaaatcttcaggatttctggcatagccttttcttttattttctctacaaatggtcttggcgctattttgccgtcatttgtaccattttccagtattcctgcatagtattcctggctctcaagttccgctacaatagcaactcctccattagaagaattttcactcctcacctgtccgttccaatgcatacggagattccctgtccgtctagcgggtgcttctcctggtgctgatgctgtatatgttgcatgtttggtaaacggttttctataaacttttccacttctttgtccttttaacacttctaattcagcattcctaattgcatttactgcccgaactcctctggaaattacttttcggttaacttctgatacctgctcttttattgttgcatgaatcgcacttccagcacttccgttttttccatctgcccataatttcattttacatcctgcctttcctccgcataatatattgtcgatatgccaagcataccagtatcatcaatatcaacaatatagaacacacgctctccaagtatcagcttatctgttctttttgctttgggacttccagcctgcacaattgtatgagtaaccacatggtctttctggctatggtttgttctctcctcgtcggaagtttctgcaagacaccccttcaatatttttgtcccatcaccgctatgactgtttgccacccttcctgtgcctgtcaccacctgtttattttcttccacaataaaatctttaaacaaattccctggtctcacatacatcattcttgtattgttcatctgctttttgccctttcattgcgctgcataccagtataaaaatatggaggcttacaagctgcctgcttactaaagtgtggtacactacattctgctgacacttcttttttcagcttgtcgtaatcttcccgccataacttcgccctttcttgcagctccaacgttagtggacccgttttcgtactgacctcataagaaaagcgcctgcacagactttccaacaacataagctttgcttttttccatgattttggataggtttcaatggcggcttggatttcctcatcagtcagcgccgttgtatcggaacaaccttctaccatgacatcgcccagctcaaatcgcatacggtcctttccaagttccttaacatttcctggatcatatgtgtatgtaccctttgccattaggtatcaactccctctgtattggttcctgtggctttattaccgcttgtagattcgtttgaagctgctttaatggagaataatttatctgcctgctctttggctgcatttttaaccgttttacggctgtctgctgcatggagaagtattaatacattctcactcttaacatctgcgattgcctttgcaccttcatcaacatttaattgcataatggaaaatacctgctggatttcctcggattttgctaaaactgtcatatgctgttcattttctctatcagaatctatgactatatcgatcatgaccgtttccatcaatgtttctgaatcaacttcctttcgttctgcggccgactgctgcaaagcttcctgtaattctttctgcttttgctccatttctgtaatcgtattattaacagcttcctctattgcctccgcaagcatcttgtcaacctgctcttgtgtaaaaagggtagcagactgtttatctgctacccccatgctgtcatttacaatcgtaattacacccattttctcttgcagcctggcatctgccacaagattttctggtatttcattgccaatgtagaatttttttcctccaaagctgcaaggtttatttgcaatcaatctcatagcgcgcctcctatactgcatctgcaaagaacatcgccaaatcgtctgctgtctttttcatatcagcagccataagaccctcaataaattcggaatgtgtgccattctctcctagataacttagaattggcaaaatattgccattccccagcatatcccatgtaaagatataacctgcggaaggttcatcaattgatggagaatctgtcgcataagctaacaggaaagcatttggatcgccaatgtattccatctttgcagtttgccccaattctgctttattcataatggagcgctgcactgtaatcctgtcaattccaaaaagctgtgccaacacatttaatgtaacagaggcaggatttgctgtagagcctccatattttactctttccagaattgcagggtgtttctttagtgcgttaaacacattaactcccagcgctaatctgtttggcatacgtccagtagattcttccattgctgttttcttctcatccataaaggacaccggatcggaattggcattgctaaacttaataaactgtccagatgtcggtatggtagaatcaacccccgtaagttcctgtccccatacaccctttttgaaaaaactacgggcaaaccatgcgtcctggtggatatttgcctgtgacgccattgtctttgttctctgctgttttgggtctgttatccttggacccattctccggtttaagtctgtctgcctgatctggtcaatacccataatcatctgatctactacacaagcataagtatccatatgttcggaaagtactgctggatcgactttgccgtatgctggttttctatgccagttgtcacgtagtaaatcctctttatcaaatatgtaataattatcagaggataaagacactgggcaaatcggaaaaatcgtctttgcaaaactcttttcatcactctggtaatatgccagcgccatatttgacaacgctgtgtgtggtctgaaggcaccctttgcaatatctgcttgaatgctcgctgctgttcttttcgccatttttcattccctcctgctttaagctttttgatatttggcaatctgcactctgcaataatcatcttttgcaactgttgaaagtgcgatacctactacatagttaccggattctgccttaacagccaaaccgttagatgctgtgacttcattccctttggcaatttcgccaccagctaagatatagccaatatccttaatctgaatatcaacatcatcaccgacatttacctttccagattccacaccggaaatatcattgattccagcttcaatcagcgcaacccctattaaaacggtagaaccatctgccgctagaacaacattgccgtccttatcatatgccataattcgatttctcacgtcctcgatagcagctccagcctgctctacaatcgtaacactctgattcacctgtactccattgaaattcctgtttcccacttgtttatcctccttccttaaaatcctgcctctgtttcatactcactcataagttctgggtgatcttcccaggctctcgctactgccgcagcataatccaaggaagaatttttctccatataccctttggcaatggtttcaatttttgcctcggctgctcctgctgtagaaccatgaccggactttccaatttcagagaatacgccggatttttcaaccgctgctactgcctggtccaacacagcaatcatatcattgtaggcagttcctcccgcagctttaagacttttcagcataggcaccaattcctcttcctttttaccgataatcacatatctctttgcaacctcggtcagttctctagcctcggcatcttcgcggaatttcttcaagctttccagctctgcccttactgcggggtgcagtcccttatagatgctgtctggttcctctgtttgtacaacagatgccaccacattaggcacaggctgggttacagacttaactactgttggttctgtagatacaggaggcaataactgttctaggggtgctgatgcgcctccatcaacagtattgtctccctcgtccactccatagcgtttttcaatgttttccagaaaaactctctccgcatctgttagtttactcttgtcaatcttcattcccatttccttttcttctcctttcggtttttggttgttatccggttcctctttttcagattcctctttagcagctttttcaattgtttcattcagcctttctacagccgatttcataatctccaactctgcctctgatatatcttcatttttctttacaatgctggctgacctgccgctggaccacttcctgattgattcctgcactactgcgtaaaattcatcaagactctcctgcattgctgctgccgcgctggtactgtccagttcatcatcattgaggattgaacaaagagaagactgcaatgcataaccgatatcccatatctcatcacaaatcttttggttgttgacttcattgactttctcactgaaacttatggaatcgcctttctgtatttcagctactgcactgtcaatttcctcttgattcataccagccgcttttccaataaagccaaacaactttttaagtatgctactggaatttttcggttcttccctgtcttttttcttcctcattttaatgtcagcatcaggatttgccccttcatctacaaaatctaccttcttgattttaagattccttagctttgttgccaccttcttgtctcctttctgtggaatatataaaacaaaaaggcatctgtttacgaatgccttttgatttatcatgaatatatattattcttatgcctcatctatctctatcctttcggcttcaccctcgatagaaaacatggtataagttccatcttttaccttttcccatacaccagaatcagtaactttaaatccaatccaccagccaactggcagcgttcctactggaatccccattgctgccattttatcctctgtaaatacaacgctttctattaatatagcaacaccgcctcgctcatgcatttcgccgccttcaccataaagccttacatattcataagctgcttcctcaagctcacagggagcgataatgtcaccctgccagtcctctattaactcaccatctgcgcgtatggatacactcgcccatccaaaggcaagacgcctgtcatcatcagattttgttatcttgaaacgtccttttagaacctcattttgtttttcaattagttcagagaattttttcactttatcctttctgcccctttcaaatatgaatataaaaaaaggacctgttcccaagtcctttattttttctgtttcttataatcatctaattgttttctgactctttttttataatcttcgatgtcaaggtatgtttcaaaatggaatagaggaaatgatttctctatatctaactccttccactcttttctcatttccaaaagttcctcatctgtacgtaaacgctgcaccatgttttccatgaaacaacctccttatatgcttcatacaaatctttcaacattcccttaaaaccctttctagttggcagtatttatcaattatctctcccaataacagatatcagcaacttgctttctcataatcaattaacagttttctaattgcctccttataaccactcgtcccattgtattcatcataattgaaaggcggaaaaggctcctcaatttcaagttctttccatctttttctcatatcctgcaattccttatctgttttcaaaaaattcattataccagacttttccatgctataatctccttatatgcttcatacaattcctttaatattccatcaaattcttttctgattgcagattcactcatatcaatacatgttatgttcgcaaatacttctaacgaacgcttataatcagatgccaaatattctggagtatgtccaataaaaagcataccattcgcttttcctcttgtcaatgaacttatcaaatcattaaatgcaaaatcactaccataatcatcgttctgtaacaatttttctaaaccatctaaatttgatattattaatgattccgtattatccattgcccgccgaaaattttcattttcccaagaatgaagaaataaataatctactctatgagcattttcatgtgttaatgcaaatctcatatcataatcatttgcattttctgcatatggattatataagataacatccctgtcgctattgtatacaaatggcaaattcagtgtttcattcttttcatatttaacttcatcatgactcataagcaatcttatactcatagcctcatcaccatttgtattaataagactactatcataatcatctattgaagcatctatccactcatctccggttctcataggactttctattaattcactatgattttccacctcaatatactccacggcacaagcacaccgcgggtgcgcgggtggcagcagatcttgtcctggaaataacattctgcctttaaacggaaaacttttgtccatttcaatttctataccttcaagtgcctgacatgtgctgcataccctgtcatctcctgatgtgctccatctcttttttactgttccaagatagccctgttgttgtgcctgccgtattccttcatctgctcctctgttatatgcaaatgcactttctgtctgcgctatagtcatagctctctgacggtgtttcttctctgcatatttgcaggttgcatcaagcgcttttctctgcatattttctggcttcattcgtgggtgctcttttttcagattctctataatactgtcataaagtttagctgctgcctgtgcatcaccttttgtcaagccaatacaagggcggataagtctggatagttcatcaacagtatgcccctctttcatcttctttgccaacagaatagagatagcttctttctgttcacttgtacaggctgtaaccaattgcgcccctcgtgtctgaatccactttaacatgcctggtgtctgtgtgttaaatacaaatgttttgtcatctaaaataggctgtcctgatgcaccagcaatgattgcatttgtccataaacctttcaacctgcctgaaattaatttggaatagtcctgtctccataattctatggcttctttacttaatgttccatctagtacagcctgccgcaactcctgataagtaattgcattctgctgatcttcccaaaagccgcaaagaatctgtactggttcctcacaattattttcaaggtattcttcaaggcgctccaaaacttcctggctgttagctgtctttactttctgtactcgttttggaggaatgattcgtaatcccatgtgctgcttttcctttctgttctggctgcttttttgaagctgtgcagcatactttatatgttaaaatatcagttaccttcctaatcgttttttagcttcttccaccttattatcttgtatttcctcatcaggttcggtacccgcggctgtttttggctctggcggctggttctgctcctgctgttcctgccttgttcggtcaatctccctgacatcagaataagttctatctggaagatgtccgacctgtcgaatgtaatcttctaatccatcatcaggtatcaaaacgccaattccagtcatgtcttttataaatgctgcgacctttgtaatatctgcatcttctatatctccatgtgtcattttgggatattctgtaatgccagaaaaatactgaccgttaatgtcaatcaatgatggtatgccctggctgttaaatgtctcacagataatatccagaaacgctcctatagcaacagcaaaaagctctgttttatcagaactcaatgcccagctacctgtcttgtcatgccccagaaaaatgaaatctgccaatactgtcatagcgattctggtatcataacgatttacaatcgcattggtgtcaaattgtcgcgttccaccagaacttagtaattccaacttataaccaaacggaaggacaactccctccatttcgtccctgcgaatatttctgaccatactttcaagtccagctagtatatcagcattttctggcatgtttttgtcccataaatctacaccgtctggtccatatagcactggaagtcctgccaaatctcgctcaataccaattccttctatctcttggattctccgcttgaagtaccatgaccgataggcatttctaagaatactccttccttctggattgttcttacgactttttgtccgaaagagcagcgccttctccataggaattgtaaatgttccaaaatctgggggcggcatctgtgtcattccaagcagattatcctcattgtcatattcccactgataaagtgtctcttgtgcccttattggaagcttcttccaaccaataagaccatcattaaatttgctctttgttcgtgtatctttcgtatttcccatacgtcgcttatatacaatctcatgaaagctccaaccataggtgagaaaagacaggatttctgatattgtgtcaatccatgtgtcctgcatatcgttcatgcagctttccacaaattcagcagcttctttatcctttgcagtatctccgcctggctgcacattccaattgcattgtcttaccagcatttctatagaaaagaggatagcgcctactatatcatcattctctgacatttccctgtatacctctatccctcgttttccccttagctcatagagaaattcttcataaatatttccgccatagcgccgctgccctatgcgacctatctctttgttatttgccatctgttctcacctcacttcctccaataactgctccttcccagattactgtctgacggtggcgccgaatatgtccagccgttttctatttcgctgaaagcggaactgcttgcatctaccatatccttaaactgggattgtggaaagctctcaagctggttgaaatacatctcattccagtctgctgccagtatatctacatttcctttatccattccttccagtccaagccactgtgccgagaatggttctgctcttgtaaccttatcccctgattccggcaatatctttacagcaaatcctgacagaaatttcaaaaagctctgtgcctgcgccttgcccgcctgtcctgggtcttgaggaaggcgtgttattactcttccatactttgccttgtctgcaatgcaggtcattttaatcagctctcgtacttcagcggaatccaatctgcggttaatcacatcagcaacaatatagcgtccatttttgcgtttaccgattagcacaccagccgtatatgcaggttctccgtcttcgtcctccgaggttgccgctaagtcccagcctctcgcccataaaaccacatctgtaggtaatacttctagcatattgactttagttcttttaaacattagtcctgctgctgcctttatcttccagttgcctctaagtaaccgttccatgtcaacctctgtcattgccttcaagtttgccaaatatccaggatcgctttccatcaagattttattattttccagatagcttgcaatgaatgtaacacttttgcaatcctccacattgacctcatatttctttgctaactcctcaggagtgttttcccaatagatgacatcatttagcaccaccatccaccttacttgccctgatcgttctgggattgcatagccagtttcctggtctatccaccagttaataaactctgctacccatgaatccgcatcagggttgcaggtcgctctcacatacgggcgaatgccacaagtagttctgtttcgagagagcatatacagaaactgatgttttgtgaaatgtgtcagttcgtcaaaaccgatataagcaatctctgtgccttgccatgatttgaggtcctcatctctttcaatgtgagcgaaactcagcttcgcaccaccatcaaacctccaatgtaatttcggtgacttccgtgagtgggcatctggaatctgtccaaatatcttattactggcgtcccacaaaccgccttctgccgttatctgattgtagttatggcgaaatatcacagcaccaaaacctctgacatctttgtgtcgaagcgactccaataacaaggcatacgtcttccctcctccagcagcacctccgtatatgataatatcagccgatgatgccataaactgcgtctgaggtccagcctggggtgcaagctcgaagacatttttcttatcccttccgttgtttggcagattaattgctggatatgttatctcgacaatttcactttcttcctctggtatgaaactaaaacggttcaaatctccagtcaattctccaatcaaccggatagcagatgtgtcaccttcaaccaaagccttttgcataagtcggacaacaaccgctgtttgataattctgatctccctttttgatgccaaaggcttccaagttgagcttatttttttgtccaaccacctccattgacatgagaacttttgctgtctcacgcatggttttttttgcccttcggacctccccagattttatgccaccgtttctgcccctctcctttgcttcttccttggttcgtaccggcttcaagtccttctgtgccacatctgtcacaccacctttccataggcacaaaaaaggaactcattatagcctttttcagcctttttcgagttcccttgatggatttatttcctattcaatttccatgcttctattccccatacagacaactgactacttctattcccttctctattgccctgggtatgtctattcctatctgcttatagaattctggatgcactatgcactcatacgcccttgccattgtatctctctgtgcctttgttacatttatccgaaagtcctttgcaatccgaagagctttcttccattccccagcttttactgcttcccttacgatatctgactgctttaccaactttccatatcttactaccatatggacaactcctataatctgaatatatcaacatgggaattgcagaatgcggcagtctctccatttcccagatccaataccagatactcgttatattcatctcgactgttttcatcagtttccaatccgatttcaatagcccgatcccctgtcactacttcaaatccttcaatctgatattcaatctctgctaccttcttgctctcctctttatacttaactccgtcatatactttaactttaaccttcataagtgctaccaccttttctttgttatttatcgtactcgtaacatagcacctacgttttttgtgtcaatagatatttataaaattttttatataaaaaagccagggcaaattttacactgtcctagcttttttcaaccatttcttcatgatgtaaaaagtacaaacaaaccaacaacgactataacaaaccatacgattgtaactacaagctttaaaagctcttttataacctttttattcatcttgattttgcagatggactatgatatagttgcattggggaggacgacctcccctttactttattggatactctcaataatcattttaattactgcaataagagttccaatttctaacgcaagctgaacaagtgctcgaatcactttagtcagcttgcgtatttttttgtccatcaacaacctcctccttcctgtaatatgtattgtttaattacaaacgtactctaacacatacaccatcattgtcaataaatttttcaagaaaaatcatcttcataatcaatactgatctggctccagctttcttcctctcttgaactgctcgaaatctgcccattccttattccccactttaccttgatttctcgcccatgtatagtaattctcccatgagcagtcatcgaacagagacatctgttttggttccagtgccgcctgctcctcaattgtcatgtctaggtctgttttaccgttccttttcccagtgagagtatgaacatcgaacacccaatccggtatcccttccactcccaggtgacatttcgtaatgtcgatatgttcaatttcgctttcatccagaatgcgatctggcaacatgaaattgcaggctacatagcaggcatcccgattcttccttgctaggcaaagaagcgtgatcgccttcgctgcaaacagagggtccttgtcatatcccttcctgcctttgttgcagaaatcatctgccagcttcagcgcaattacctcctttgtcataataccatagcagtcctccgctgataccaccaccagccgcttccacatataggtgtggaaattcccgaataattcataggcagcatatcccgccaggtttgggttgctccgtcgaattgctttctgtagcatagatgccatgtcgtacatattatgaccattcttagtcatcaaattataagccatgtaatcaacctcctattttttatcgtaatcgtatcgtaacacctacaccaccgttgtcaaataggaattgacgaaataactgaattatcttggtacatttatctgaggcttgttattcttgtaattgtacttgaaatacttcccccatttccgcttcatattctcaatactatctatctgatcctggcgcagtttcgagctatctcctccggcattgacatcttgataatctttacaaatgatatatcgtggcttcagaataatacgattatatagaagctcctgtaataccaggtccaaatcataattatatttacagttttcatctggacgggcttttaacaccttcttatacacccacttcaaagaaccagacgtacccttaaaggcaaattcaccatcatatccatatggaataccagtagcatctatacaggcataaccgacattcagatcaaccatcatctgtgcgatacgctcaatctccgccataatagtatctttatccttgttcaatcgggtggtgtcgtcaagtcggtacatcatatcttcaatatcatcatcagcaatgaaaacaatatcctctggtgcatggtccacaatccaccaatacgttcggatagcattgttaacttcctcgtctggaaccgcccacacattctctacacctgccttacggtacagttccgcttcgcttgccctcaccacataaacacactgttccagtagatcctgagtgagtattttattgtaccgatgatagctctgaacataaattccaagcgtcaatttatcttcttttttcatctgctatcaccttctccatctttttaaaatgactatcgatgtcatgtccttcattcattgcatagtctaggattgacatataggggagaaaatctccccatagctgctggtactccatcgattcccattcctgatacaccagcttaataccattctcctggaaatgaacatctttctggtagttcctacctccggttcccgataaataaacatcagcaccaagacgtttacatatctcaatcaccctggcttctgctctgctttccagtttccagtttgatgtgtaataaacctttttctcattccagccaaaccaaaacaggaagaaattcatcagtgcaatatttagctgtgccaggttatcataacatttattgacaatttctttataggcaggaaaaagctctgaaaaatacggagccttcttatagttcatctctaccatctttatgtgtttgtattgccatttaagatcatcattcggagttacctccaaaagtgtctgaccgaactttttatgcaacggaattttcagtcggcactcgccctgtggtgtcttgattcggttccattcaaacacatagccattactatactgtgcatcatccaagaatacaaaagcatccgccttccaaatcttgtggaacactcccaaatatggcaaatagcatggttgatgtatcgtaatcatcatattttatacccccaatccaaacaccatctgaaaagactctgccattagcaaaccagattggcttcctcttagaactgccatagcattgataacttccctagaacgtgggtgaggtgctttcctgataacatctttatagacgcctactgccttaactttctcagacaaatctttctcacttataccaacaaatatattcggagtaaagaaaccattcgtcgtacttacattccaatccgtagaggaaggaacctccatatacataatttttctgattggctgaatatttacaatctgtctcattgggagtttcgatgcttctatacagcattctgatgtgataccatgatccacatgaacatctgctggatgatgcgtaatcagaatatctggttgacaatctttaattgctgactctatgaaccgaacaattgcatgatggtcttcatctttgaacctcatacacccaaaatctccaatataggactttttcactccaagaatggcatggctagatgcaataccatcttccaagttatcatccctggtagggctccaatgactaagtagacaaacataaacttcatcgccatttgtgactgccttgtgcatggtggcgccagctcccaaaacctcatcatctgggtgcgccgctactaccaaatatttcatgcctgcttctccccttcgtctttcttactattttctaaaggcgattcgactttctccaacttttctacaatctgcgccttcatctgatcgtaccagattgctctcgccttaatcttccgctttttcgtaatggcaactttcctaccctctatccccaatttgcgaataaggtcattataatcaatctcactcttgcaaactatcattacataattgtacttctcgtaccgaatcagctccatatcctcaattttccgttcttctggactttcattgttgaggtcaattccgagatcaagattaaggtccgcagtccaatctgccagcaactctaaatcccattcgccagcgtgggtattatccataatatttatagagcgaagttctgcttcagaatatcctatcagtctcttaacatccacctcaatatctggatcacgcctagcaagtatgacggatcgttggtttccggcaattacattgtcatgttcatctacaagaaaaatgccgaagtcaccgaacaagtccattgaccgttccagctcctcggttttcttctttgaaattttcctagggttgccaaaattgtgtttcagatcgccaaccttcattttacacacttcaatccttttcatagtttattatgcactccttttatgttatagttcttctttctttccgatagcaaaaaagaccgtccactttaagacagtcctgctcgttaattggtacaccacacaacaaaacgctcaaggaaattatccttgggcgttatttttatgttttaataaagaaaaactatatggatttgtgtcggtggtgacatttgattggctaataccattacaccacattctttttctaaatgcaacctactctttttctacctgcaatttatagtcgcaaggaaggtttaaatcatatcccaagtaggtaaacagccagtattccacatgctattcctaaatctttatataccgtcttttcacttatattttctaccgtagaaatttcttgtaccgaatattctctatcttctaaatacatcatgctaagttctctgtatcgtcgttttgcttctttatttccagacttttcgcattcttcatgatacagttctattgctttttcaatacgataaacataatataaatcttcctgtcttcgtttctctccgttctggattattctttcagatttacttactgtctcccttgcattccccataaggtcctgtatgaatttccatctaaggtcaatctgttcttcttccgtgaatcttgcctcatctgacaaagtagccttaattcttcgatatgaactgagcatctttttcgttttgtttactttattttcttctctagctcgtttcttctctactttggcttgttctgccctaaatgtttgcaccgcttctttccctgctatagcagcaatctggtttatttgttcctgggtcaaaacatacatatgtatttcgttttttgtttccatactgtcgcccccttgacttatcttttcttatgagctataatgctcatatgtcaaagggctgtctcataagaaaattggggcggctttttttaattgacattgtactccaccaatctattcattttaacatcaactctctttcgattggttcagttttacagtcattcttatgcaatccgaaaggaagttcataatcttctggcaatacccacaaatgatacattccggcttcgtcaatcaatctttcctcactcggaaatacttctattgctgtccgctttcttcctgccatgtcattcttgattttttgtttttcagaccaagaaatatctgtattatctctatttcttatgcagaaatgttcaacaatgccaatatttgttttgattaggcgtgtcattacaatatattttctatcaagtcgatagcatctatctaaatctccgcaccaaccatcaccatattttttgtcttttggtgataattctcttatccatttactcattccacttctccgcaaaccacttcaaattttctttatgacatttatcgctttaaaggaaatatctttgtttccaaattctgctaaagaaactaatgctgatagttctcttgcatcatcatagaaccttctgttatacttctcattcttcattctttttctttccatgaaacgaagataattctttgctgtttctactgtcacctttatatttctcctatttacttacaagctatttcaatgttaattaatcttctgttcgcaacactccaaccgaattaataattgcaaaaagtctacctgaaccttttgcaattataatttctccgttctgaatggcatccttcaattcatttgttataatttcttttgtgcattcaattccttttacaaatcccattctgtggcttgtctttttctctctttcaagcctactttcatgttttttcatttttaacctcattctttttacttacaaatcacttgatttatactcttgtaaacgcttgtttgacaaatcatattttctcgattattcgagtttatttataaagccttattccacggtgtgaaatttcgtactttttcaagcaaggtttctgggtgtccgtcaaaacagattgcccgatcatctatataacaaattgcaggtggtttttctttcataactgcatcaacaacgatattattatctgccaaatacttcttgaccgccgacattccttctggacatgcacatctagtagaaacaataatcacttcgtaatcttcttgccttaaagaattgatcgttgattgtattccttcaacaactggatctggaattacatctgctcctttccaaccgcttgtgtaactgtgaattactccatcaaaatcaaatactactgtctttttactcattccctattcctttcttattgtcttcttgaatatttttattttactgaatccaagcttcctgtggcagcttgttcaaaaataaatctagcttatccatttctttcccaaacggatatttaaactctattgtgtctttttcagccttgttgtatagttcttcgatttgttttaccatttggagataacacgtattctgatctcgcaggatatccatagatgcctttaggcaatcatatctgtatgtaagggctataaaagattgataaaccctaaaacaccgcatggcagcttctatcaggttatcttttgtatattgtttcaatatttttcgagattgatcttctgctgctgtgtcgctgcacactaatctaaaataatccccttcataggaatcatgtcccaataaccctccgtaatcttctccggctcctaccgccacaaaaaactggtcaaagcactctggaacaaattcctcttccaaatcccaccgcagttgttcacactcgctacataaatctccgaacatcattttgaactcatatgcctcgtcctcgtcaccgtctaaagcattaataagagtatcatcttcatcttcaaaataccaacgaacatcttcacatttcccttgaatgtcaagtagctcctctttgatactatcaagattcaggtttctcacaattggctttttatatcgcaattgtcttgctttttcccgctttgtcgcctctacatctatcaaagcactttcctcctcaaatccttattttaacgattattcatcaactatcaatgtaaatatgtctttgtcactaccttcatcttctcgcatccaacgtactggaaaaccttgtattttaaggtttgcgaagtcctcatttattacatacgcgggatttaaaataatttctttgatttcactactttttatttcttttctcataaaatcaccaccaatgaaatttcagtttagttgattaaatccttatttactcaatatttatagtcttcaaatcttttagttttttcaaatactgccctcatgtttacccatctttgcatttttcgtacaatatctgtagattttgtatgctccttgtcatatatcatcacataaggccagtaacctaaatctcgcaatatgtatatgtgctctaagtcttgctctatcgtggtatcaaagtttgtcaacacgtatactgtcaatttccgtttatcccattgagtgacccgcttaaactccttgaattttggaataactttctctttatccccgtatctatcccacgcaaaatgaatgtttttcgtcttgatgcgctttatcatctccgctttttcctctgtcataaggcgaatatcaacgccttgtgagaaatccacccatgatttactatcaataagctgtccaagaatatcttcccaatctttacacgctatggggtttgggtcaagcaagacaatgtttttctgccctctccaaaactccgacaaatcagctactttataagccttgtgcccttcttttgctttaacatggcagaaatcacaccctctcgggcaacctcggctcataaatccataggcagtatcttttgtcaattcggaatatagactgtaatcgggatatatgtgttctatctcgtaggaaagttgagaatcattttctttgtgataaacttctttaccatcaactaactttatacaatatccactaccaccctttatcacttcatcagcatttacaaagtattcgtaatcaggtgtaaagctgaatacttttgacatatatattttgtccataggtggaaagggaaaaccatgttgtgttggtatgtaccattccacgctatctccttgctgtttatgccatgcagacaacttcatcagtggtaaattcgggaaattatggctgtcaacatctatcaatccaattctaatattttcacccctaaatcctttttcagctccttatcaggttcccaattccaaaatccctgcctgccttttgttggtattggttttggcagcagcttaacattttccagcttccatgcataccgccccaacgtgtagtcaccaagagctagttcgtcctctgtcaatgtagtaatatattctgatgttatctgaatacaatccacaagatcggcgatagcaagtacataacccataggaaaggttacaggaccatcaattatctctggcagtttcattcgtctaataatcacttctcgtgcttcgtcgctcatgtagaggttgctccatgtatgatgtattgccttcatggcagaatgaatcgcaatctgtcccctgtatttcgtggaccagcttcttgtctcattcttctttatacctgccgccacagctcctgcccatggctgccatattgttaatgctttcatatgtcactttctccttactatgtatgacgatagttataaattcctgtaatacaattatcaacttctttgcggttattggcggtagggttcaggtaatggttgccatgcaataaccttaaattttacccctctttgcccaccattccattttccgtcaacagtatgcagcgtttttgtttttctttccccgttctcaaattccactgtgacgttcacatcatctgaagttttctcaaacattgcgctatcccacttttcggtaccttttaacttcgcaaacatactgtcatgttcttctggtaaattcttcccatcaccgcagtatatccacccgctgcaatcctctactgactgctccatatttactacttctgatttttctgtaccacttccccatttcagatgttgaccacatccaggacaataagcatatacatgacaaactggctctccgcaaatagggcagattccttcatcataatcttcatgttcctcatattctaacggtcttttaggagttgtcgccgccagctttgcggataaggcttctatggtgtcggcggcttctctgaacgcttttacaattccgtcaacggcgcacccacttttttcaaaaatatctgccttataccgcaattcctttacctgttcgccaatgctcattctgtcaatcctccataatctccacaccaatcagcacaactttatcagaatgccctatatcatcaatgcttaattcagcattgtatgtatcacaaggaaatttatatgtccttttctgcaattcctcctccatgacttctaaatgcactcgtgcttctatgtcaaaatctctgtacggctctaattgccttattagctctgctactttcatttccttatcctctccttaccatattgtcagacacttctttctctttctgtacggtctttaaatccaatacccttcagcatttccgcaaattcctcaatcgttggttttagtccctcaatctgaatcctgcccataaggtcaatcagttccggcatgtgacttaatacttcccatgaatcatcataggcttgcaactggattccgacattatcccaaacaattttaaattccccgatacagccgccgctttcgcagtacatgccaacgtatatgtgtggctttcctaaaactatatctttgtcccatgcttcaaacggcatattaaaggcttttatatgttctttcatacctcttatcttatcctttctatccctttcgctctccggtatggcttgctgtattctttgccgtgggtgatggattggttacggctcatgatatggctccagcaattcacgccatgctatcacatcagctttattttgtgaccaattttttacgccgttaatgttagaagtaacagcccttacgcacacattaccctgtttatcacagacaagtacaggtcttcctacttctggcattctccttttacatggaatccactcttcgctataatcttttcttgactgctccatatctgccagctttgcggatagaaattctatggtgtcggcggcttcttttattaccctgtctttctctttgtgtggtgctccatattcgtaaatatccctcaattcccttatctgttcgttaagactagctttatctttttccaccacttcaaagcacttttccaaccattcaaataaaaaatccagattataagaactatacccaacatgaatctgcccatctgacaaatcacggtattttatttcataatatggtttcttctttgtcccatgcacgacaatttccgcactcgatactcttatcttttctatccatccgttcaatacttcactcatttttttatttccctatcctttctttaatttcaatgcagcttcttctctgataaggaaaactgtttttccccattcacaaaccgacaatgtaatagtttcctcacaccaatcttcgggacttaaatcattttctgaatctggtttcctgtatggaaatattttttcattagcatcaagcgtaaatactatatttccgtctgcttctatctgcactccgcaattaatgcattcgacaatttcatcttcaaaaatatagtaaacgtaatctccactgctttcatatacttctgtacaggacaattttagaattttcccttgttcctctaagtcctcgtattctgccagctttgtaatggctgtatcaatatcacaatctatagcatatggcggcgcctgctctgctgttgtgtttgtatacttcccatttgttttgtatgttaatctctccatatctccctttccctccacaatctcccaaaaatctaatctctctgaatgacctcaaaatcgtctatatcttctatgtcctcaacgtcctctatctccatattgccttgcacatcctcatcaacaatcacataagctgtgcaggacatttttactttaacttttctctttctaccatatagtaagtttcttcgtgtaatacttggtaggttatggagtatctctccaatcaataatctgcccgcactttgaccagtattctccattcagttttgcaacacttttccgcaattagggcaagcgtaactataagatattttcttacctctttccatcttgaccaatacttacctttacaacttctcatacttttcattcttcgcctccaatctttataaagtcctccaacgtcatctggctatcctcagacgcaaaccttgcttcaaggtcatgtatcgttgttccatctggccgaaaagttttgggactatccctgtcaagacgaagcatgacttcccatttgtcagggtggtttttcctcaaatccctcaactgattaagtgtttgataatggcaaaaccagcaaccatctctcgaaaaactctcatagattggacttaataaatcattcttcttgcaccactcataacaatctttttctgtcaatcctgcttttacaagaggatacatgtttaagtttgtgcaatttatgattttctcctgccgttcggttttcttttcatctgtcgcatatccgatataccagaaactttctcctaactgtgttttgtacttctcaatggcagacatttttaagtcgctgttgcaccatgccccacgcacaataggaaatccgtatatctttcctctgttctctttctttgccctttctccgcggacttgataaaatctgttttcgtaggtcaaatctgcccttaaatgctttatctcaaatccatatctgttctttataatttcgtctgctttctgtctaaattcctcaacttcaggataataggcagaaattgtattgttaaacataacatcaaccgttacaatttcatcgagtggaagtttcaaaacatttaaaacaacatctatcgttttaaggttatccttcccataactgcaacttgcaataaatttcacaacaccacgctacaaataccgtatcgtggatatacattcgtttgctcttatacgacaataagagtttccgcttactgccttatagccacggtgttatattttcctgtaccacacctcaatcactcggcacaaacccggtttgccgggattcgttattcctttccatcaaacatttctgaatatgcctcattcatttttttcataatcccaaaatacaaccttgcacaactttgagttacaagtttattaccttctatttgcctttttaaccttttcaatctccgtgagggaaggtgcattaatacatttcttcataccctcatataattcttttgctcccacattgcctctctccacttcatctgccaggtggcgtagaaccaatacaattagtcctgcgtccgcctttgcatatggagatatggcactgataatcttttcggaataatgctgcaaacctttttcaaccagcttcatagcttctggcgtctttccatcagcaatcagcttatttccacgatctacgtaactgctcattctctctttctgaaatgccataattgcctcctattcttcgtctatattaacataaaatcttttccgcacttccaacaattatattttgtatgaagatcaatgtaatgcccttctgcccaatttggacttctccacatatcttgcctcccgtcattttttcatttcatcaaacagccctgcccatcaactttgcaaatcgttcttttaagcgttctggaataggcgatgcactctgctcggcaatttttctttcctcgctaacagataacgtctcctgattagtctggattactggtgtttgttcatttgaattgcctgtcagttttaaaatatctggtggaagtttttccaatgtcttttcttgggataactctgcccgatatgttcgcatgaagttagactgtgtaacgttcatccacgttccatcaacgtcctctgccattgcccattcacgaagctgtccaggacttacaacagctttctgtactacaggtggtagtttagcaaattcttcctccgaatgataaatggaatttctcattgcttttaatacaagtccccatgctgccatttcgttaagctcctcattgccaataaataactgtaatttctcaacaatctgtccaatgctgggcgcaaatccacttgtatcagaaagtatatatgctttcaacgccatattaacctgctcgtatgtatatctccccagcatatctgtccatacctccacaacaatatctacattaattggcttgtagttcggatacgttgccatcattacagctatgacttttttaacttctgttttggtcattttcttcatcctcctttatactgtttaaaagatactcaaattgtgactgacgattttcagaaccgccatcccttgatgacgaattgtcatcattcaacggaaatagccctatccaattacgaaaaatagactgttccaatatttgaatagcaacttcattatcgccctgagataacttttgcagcttgctcattgctagactgatcgctctatcagtcatagactttttcatctggtttcgcatatccaaaaaatccataaaagtctgattgagtttttcatcattagggtaataaatttctacctttgattttttaggtatcttatttttgcctttactattatttttactattatctttactaatatctttattatctttattaagatctctattattgtctttactatatatatcagtaaacgaaaacgttaccgttgtatccataccatttgcatacggtatagataccgtatctgcattagatctaatttctagtaaataacgcttaaattccttatctttaacttccctaatagatttgtctaaacccgttaaaaacttttcgcttttagtccaattgtatttataccagttcaggagaagaatttctttactttcttttgaatagacaattactttatgaaccttttcaaatctttcaatcagtctttctattgtatctctgttatatcctgtttggtttgcaatttgcttcaaactaacttcatagcatccacataaatttgtctgtggatttgtaaacaaatatagataaaaatacttgtcttctggtgtaaaatcatctgccactttattatctgtccaaaatgacaaatgaacatttctgtatattgccatttgcgatttttcacctcttttctttttgcatatatgcatctctgtgacgcactatttggcgcggagagcgtttaaaagtgcttttaggtaaaatataatgtctaatactttttcgctctctacggggctatttcagcctttattatcatttcggttctttacatttcttctggttctccaaaataacgcttatatactgtttcatgtattccagatggatatttattcataaatgcatgtgcctctttctggcttaaaactttatattcatcatctcctgctaaatcataagttgtctgtaaaataaacgttttgccttttgcataatataaccgctttgtctgtgtattgaagtcaaaaagcgctccttttctgctgattactgcctcacataacaaatcctgattggacagaacatactttttcccgttaataattatgcctgccatactgtcgtctccctgcctctcaaaagagccacattgtcaaggagtttcattgcagttttaaatcctgcaataaaaccactctcttcaaactcaacggcaacattcatcatcttgtcatacagctcactctgtagcttaacattgtcgggaaatgcacctacacaaaactggtcatacatatcagcttctgtttcagcagcagtcgtatgccttggcttatcttccatacttttaacatactcgcaaaataactctgcgatagcagcgtctctgcctgtttcttttttctctgggattgtttttgaaactttcatggtacggctctccttttctcttgatttcgccgcaatgatttgatagaatagatttatcaatcgttttggcggttgtgtgaagaatggtcgctaaactttggacggtgcagcggctattcttatttttcttgttcaaactgaacatcaattccctttctaataacatctgctttcgagatttgttgctttttagataaaaattccaatttatccattgtaatttcatctaatctaagcttaaaagtattgtttttagggttttttgtcagctttgttcctttttgaatacccaatatttacacctcactttctttgtgggcacaattaaattataatgtacccacaaagaaaagtcaatagatattttctaattttccaaagttttctaccacaccttgccgtaccatattcaattttcaaagttcccaattatgcgcaatatttaatctcatattctgaaacaattttggagaaaatttctcgcagtttcttgtcttcgtcaatgatatccatgcgatttgtattctttatctcagtttttgtgcacccactatcggacattcgctgttttttgttccgaagtctggttcctaagtcacaaccagcgcggcactctaattcactgtacatctcatttcgcaaaacgttaaattcggttccacaactcttttgaatacgattgaactttgtattaatttcttcacgccagttatcaagtattggtttaacggcgtccttaatattttctgtcacctcgtaagccttagatgctacttcttttgcactgtccgcaatcttcttggcttctttagcttctctttccgcttgtgccaactgatcacataatccataaagtaatttcatttgtggagataaatcatctacaagctgtctgacacgaaaataattatctacaagttgatcttgtacttcccatgctaaatcatcagtaaaagattttaccaacatgagatagcctctctcagtaacaagatttacatcattttgctgtcgcaagtccatatttccaatcgttgtccgaaattcggacgccgttatttttataaaatgcttcccctcaataaaccgctctctattatcagaaaaccgctttctcgcagttccttctggtctgccatgcacaacatcaatatccttgaatgtcactactcgctgaccattatattcttttactgaaatgtcagcttcaccaatatgtactaaattatttgcattcacatttctcactgttcctttctcaaattcctgctgtaaaatcagcaatagttattccctttcatttcgtatatgcgtatctctatgacgcactattgagcacagagagcattcaaaagtgcttttagataaatataatgtctaatactttttttgttctctacaaaactattttccttcattgtcacttagacattcctttagcatcttcttcatcatgcaacaactaaaaacttattaataaaattctgttgtccttttccagaaaccaaaacagttttggttattcttacagaaccatcaggattagcaatcgttcgttcctttatttcaaacaagtcaagttccattgatttctgggttggcatattataatctgttcctttacgcttaattagataaccattgccacgcatccactcaaataggcgattttgtcctatgtctattccgttttgcttcaatatctttgctaactctcctatcaaaatagatgtgtttgatactgatactgcatcggcaaatatttcttttggtttcatacgttttacatcttctaacaatatagctttatccatttttagcttgtctatagccttgtctgccatttttagcactcttgccataatttgctctggggtattccatgccttttccaaatcaataagatactggcgacacatctttcctttttcgctcttactcataaggcacaaatgtttagccatatcaattgacaaattataatcgtctatagtctgtgttcctccatattgattgcctatcacatctatgtgacccctagaaaagtccacattttcaacaaattctttaaagttgctgttgacccaacgtgcaaaatcatacttcacttccaatttcttatgaagttccctcgccgatactgtcgacggttctgtttcatagttaaccttaactaattcatacatgcaatatatcttcctttctcgttatattctaatattgccatctcttttgcataaaatcatcgtgcgcacttgtgtgcacgcccaaatctgccaagtatccctcactctgctccacataacaacctcatttccgcttgtggctgttcttgtaaatcttaatcctcctttccggcagcagaaaccaactgctgccattttctgtgatatattgcatgaacaaatactataattgatagttacccatttattaagatactgccttatcttccacactttttatttttttcagacaatcttttagctgaaggcttgtcaaatgttccacgctgtctaccttatacttttttataacacttgaccatgtataaccagtacgaacaagttcattagaaagctcattccttagattgtctaaatctttagattgcttattaccactattctttctattaagttctggcgtatctgcatctggatcatcaatcaaattttctgttggtatgcaaaaagtttgaaaacaggcatacttaaatgctactgacatagctttatttgtggctttatcgccgctgtccattccttcgccaaccaccacagcatcaacataagaaccatcaacagcaaaaaacctatatttcattttgcacacagaataaatcaatgtactgccctttcccgttgttcgttcctctcgaatctgctcaataacttcaggtgcacaaaaaatcttatgtttaatcattgcaggatttaatgcattcattactgcatcaatactacggtacttaaatccctgcgttttattcaaagcgtcttttccgactgccttaatatcttccataaccccacagatagcagcataaatcataggttctctcatcttttcttcactcattcaatcatctcccattcaatccccacactatccatatacatctccagcttctcctttgcatcattggagagagaaatacgatactcatagagattttcttctccctcaatatcaggaataaaactttcaattacttcctgtgcggcatcttctcttgcctgttctacagcagcgattttttctgcttctgcctgtcgcaacgcctcttccttttctgcttctgcctgtcgcagcactgcctccttttctgcttctgcctgtcgcagcgctgcctcctgtgcttctctggctctttgctctgcaagcatcttttcgcgttcctcacgtctgattttctccacttcctcctggcgctgcttttcctgttcacgtgcaaagatttgttgtttctgctgctcatagttatttatatatgtaattgcatctgtaaggcttaaactatccttgtatatgttcaatgctttttcaacagcctctgactgcatagaggtaattgtatttaagtcattctctgttttatgtacaaactctgcaatatctgcctttatatctttttccttatatgtagcgttttcccactttggattgtagatacgctccaaagaaatatatacctgcaaatcttctggaacacatttataaattttctgaataagctgtttcttttctgcaatgcgctttttttcaaacgcctgtatctgcccattaattaggtcaatcggctcatcatacagccctatcagcttctttgcctgtgcctcgaaattctcccaaggcttcatatattgcgtctttgcatcacgcatgttgtcttgtaaagacttcttttctgctcgcagactagcaacttcctttctcgcagatgccttactatcctctgtgaatactatcccttcatactccgcaagcttatcctggatcgctttttccacctgtttaaaattacaggtaatcttcgcattctcctgcgtaattactgcttgaataccattcatttctattctccctttctgcctcttcataggcttgtctttctctctttaagcgcactctctctggcagcatagattcattccaatcccacgcttcatcaacaaccatttggtcctcaatctgacaaatcattgtcaacctccttgtaatcctcgcaaaccggaccccactcacgctgtccacggtcaaaagcactgaaagtcttaggggttatgcaaataagacttttgctacaggagccatctatacgatatgcgcaatcttcaacctcacacataattgttgtaataatcatttactttcctttccgcttatgttataataagcacagatttatttttgaaaaaacagccccagccaaggaatactttccttgggggctgttttttcttgtacttgtcggaatcctcaccgttgaggagatgcagcactctcttgtacctgtccaagcattttccgctagacagaatctttaaaacttcttgctccatacttatcccccatcaaacgccttttttctgcatctagcatagacctgtatatatagcaagcagcccttgaactaccatcttttttatcatacacgcctatatttatgtgctgatgcttcatccagcttcgtagcgtagctgtggagacacctaattcaagagccgcctgcttagttggtactagctctgctttacatgctttgaacataactccacctcctctactacaatgcccttcctgccgtctgacaggggtatgacgctacctaactcaatgttatctgactttaacggctttgtgtttaaagggtctgctgcctgctttaactgctgctgtgtcacctttctcacctccttccgaaatatacagctctctcattcttcatttctcctccatgttaaacaagttcatctgattatactttggaactttcacaaaatcgactggaaggattattccaaactgttctgaaaccatcttgaacgcttcacatatcttccaaggttctgtaccctgcttttccatgcgcctgtccatttcctttgtgtatgatgcgacttctccaagtgaggtagcttttgtttcatacaccttctgctgaatggcattttccatatcgtgaaaccgcttcacataccgagcagtaaataaaacacctttttctccggtgaacttgttagcaaggaaatcgcagccgagttttgttacttcatagcaaggtctttcttttccttgtgcatcaatatagacagatttcacaaagtaatcagccacggacatttgtccttcgctaataacctgtatgtaacctttgcggtctttcctaccctccaacttctttaaaatgtcagcgtgtgacacttccatcatttcagcgatttccaatgttgtaattgttgtctttcttaaattattcatacaccctcctattctgctcttgtctgagtacacaattctgaaaagagagaaactataaatccgtttgcttagttgtgtcttggcttgcattgtctttgtcaagttcttgtcttgctaacagcgccattgcattggacataataatcatcatgccaaccttgtctagccttgttagcttatctattacctctccaatactgtttcttttactttcagtcagcatatttttactcctttctatagatttcaagtcaaatgatatcaattattatttgcttgttaagcatactttatattatttttatgtccttgtcaagtgtttttgaaattattttgcgcttgacaatcatttttttaagtgatactattgttttaggaggtgatatattgactgttggagaacgtttaagatatcttaggaaaacaaaacttggatataaaaattgtgatgattttggagttcgtattggtctttcaggttctaatattagaaatatagaaacaggtagaataaaccttaccgaccgtgtagtttctgatatttgtaatgttttttcagtaaatgaaaattggctacgaaacggtggtgatgataaagacatttttttagaactaaatgaagatgaagaactagctatgtgtacacaaatgttaatagattctgaagtagatgatactatagctaacatgataaaaaaatttatatgtatttatgaaaaattagatactgattcaaaaaaagtattgatgaatatcgcagatgaaatgattaacaacttgaaaaaagagtagggcagacttaatgtctgcccgatagatgggttttaatcatgattaggatttgaattataaacttcttttccttttcctcatctagcttttgaatgagttttaagacttcacttttatcattcatatgcaacgctcctttgtatctaaaataaatattacatgtgttttaagttattaaaatattaacataaagcaagccgtaaaatctaccatcatcttttcataccttgcaattatggcaattttcgtgaaacacatacaaaaaaataaccgtcatttttccataatttactgcaaaataacgaaagtattttataacaaaggagaatgtaaaaaccgctcagtgttaccagcactgaacggttttaaatagatacccgaatatgatatctgtacgcaaaaatattgtatcatcttcgggggcagcttgcaagcgaaaaatcgctggctgttatttttatacactttttaaggaggattgatacaatgaaaaacccaaatggatatggaggaattgttgatttaggcaagaaccgccgacgccctattgctgtaagggtcccaaacggtgtcaaactgacagaagattgcagagaaataccacaatacaaatatttaggatacttcgaacgtaccccacaaggtaagaaagatgctcaattattattggcacagtataatgcaggtatgcctgtgaaaacttccaatgttaattcttgtcctacttttgcagaacgttataagttgtggttggacagacatctaaaacatgtagaaagccagaagggaactgtatcaaatcaattgtctggttcttacaatgctgctttcaaacggtgtcaaacagttcattccaagaaaattaattgcatcaaatatcaagatatacaagacattgcggataatgctgcatcaatgtctagctcaagtgtgcacaatttaaatatagtgctttttaatgtgtttgatttcgcgagaaaacaaaaatatattgaagaaaattttataggtgatatcgaatttaattacaaaaagaaagcaaattctatccatgaaacctttacagaagatgaactatctatattatggaaaaatgctgacgatgcaaatacacgtatcattttaataatgatttacaccggtctacgcatagaagaattgctttctatgcactgtaaaaatgtattccttgcagaaaagtatatgataggcggtataaaaactgaggctggaaaagatcgtatcattccgatttctaataaaatactcccatttatatcccaattatattgcgaaaacttatatttaataagacatggtaaaaaacgatactctcgtcctaattttttaaataatatatggaatccaactatgaacaaatttaatttaaatcatttgcctcatgacacacgatacacttgtgctactctgatggatagggctaatgttaatcaaaattgcatcaaagaaattcttggacactctaaaggtgatataacaagtaaagtgtacataaagaaaaacttagaggaccttatttgcgctataaacatgatataaagttgtatattatttgtatatttctatcatatttttaatcattttaacacgcactaagcccgcaaaaacgcgttatttaaattatataattttaatgtgaatttaaaaaataatccttaattttaaatggttaagagattttttgtatattatatgtatatttttattaaaatctttatccattttatactattcatatttgtatcacaatgtgaacactcacacgactaaagtcgcgtgcttccagtcctttagagaccagaatacttatctacaatacagatcccaactcagatattatacagtttttctgcacaaatccgcattgagcctaattaatgtagacaaggtgcgcgttgttcccgtaccataatcagagtcttttgtctggctaggcaacctcttcctcacggaatcggattttaaaagtccccgtataaaatatctcgcacctatgttgtagcttgcgttcaggtcacagttatactgcttgccattcttgaacaggcatatggagtagttgtatttcctctctccgttctggtaatactctccgcgctcaactttcccagaaccgtcaaatgccaatctgctcgtgttccatgcacatatcctgcttatcctaattccaagaatatgcgccctatgctcaactatcttctgtatttcctgtttcctccataggtgaagcctttgctttttacttcctctctttttgccacttgtgtcaaggtgttcaaatacaatgacatccgcattatgcaacactacaaaatctataatctgttttgctgtctttctgctgatatcagtattgatgtcattgacatgcttccataatataggagttttcctcaccccattttgctgtactttctttaccctgtttaatgctttatacaaatggtctttttctgtcgcaagattgatgaattttcttgcagcgacagttccatcagactgcattattgagcatgtagcattattattaagccctaaatccacagaacagatcgtctggtcttgtatgtccacatcctcaaatttcaccttgtcttcaaagggaaataccagataccagcatttaccttgtttctttaatgttggaacatattccttcttgaatgcacaatggctttttatgtatcttacatcctgattgttcagtcttatgtccagccaaatccaatcgttcctatggaatatctttatccttgccgtatcatctcctgtgcgtatatacatattgtccttatataatgtcggcattacattcctgtcaagctgcaatctaggctttcttcccaattggtttccttcccagttctttaagtttgaccgataggatgaatagcatccagcagcagtattgattgcagacctgcgcaggtagcctggcattttataaaacttactattgaaatcatacttaggatttagattcttctttgtatgcaaagataattcttcaatctttctgcatcgctcaagattcttcaacggttccaacatatcccattctttatcacatatatctatgaagaaggatactgcttcacgatatattcttactgtctcttcaaatatcttgttgtagttctttattttgactttgtatgacgaaaatatattcatagtatcctccttcctggtggtatatgttataacttctatattctccgctcacttactcatgactgaagtcacgagtatgcgttcgctatttaatcaacaacatgcaaaacattgcaaatatcgctttttttgacaatcgcgattgttcgtatctcgatagacgaaatgcacgaaagaacttgtccttgtctttttctgcgccctttatggtgtggataacctctttgaactttctctcgtctctcatgcagttttcctcgctttctttttcatttcagcctgtgctacacgatagccctttgcgtagccataccggaaaagggaaacaatcgcataaaacaagccgtttccatctgcggattgtatctccttaaacaacgcaaacacatcttctgttgtaagcgaatagtttctgtcttcaggtttgctcttgaagaaccgctctacatacttcttaatcttgtccataataagattctcctttcgccaaccctgcacctactagacagccaagataaaatagataaacactgccaaaatcagttatgtttttaggtgtcttcttttttctatcataccctgcgcaatatatctgctgcaaaagcgcttcatcataattggcaatagggatttcactgtctatctgtttttcaatcagcggaataatggtctttgctgtatcctttgccttttgttctgtcttctgtttcattcgccatgtatgggcgtctaaaagctctctgcatgtgtggtgtccatatttataatacacataatcaagaaatggaccttttgcttcattatcagctaccccatattctggaaataaaatcacttccaagaatccatcaatatcttcttggggcactttcttgttgacaaacaaaatctctctcttattgtccactattgcgataaactcatcaagcttcgcatgatacagattcttcaaacgaaaaacaaggttcgctgaaagttctactggttgaattttctttactaccataataagattctcctttatctttttacttccccaaactctcaaacgtcaactctgtgcctttctaccttgtcaggttgtgagctatgaactcacaacctttcttggtagtgccatagcaaggaagtattctgattttttacatttcttctggttctccaaaataacgcttatatactgtttcatgtattccagatggatacttattcatgaatgcatgtgcctctttctggcttaaaactctatattcatcacctcctgctaaatcataggttgtctgtaaaataaacgttttgccttttgcataatataaccgctttgtttgcgtattaaaatcaaaaagcgctccttttctgctgattactgcctcacacaacaaatcctgattggacaaaacatactttttcccgttaataattatgcctgccatactgccgcctccctgcctctcaaaagagccacattgtcaagaagtttcattgcagttttaaatcctgcaataaaaccgctctcctcaaactcaacggcaacattcatcatcttgtcatacaactcactctgtagtttaacattgtcggggaatgcacctacacaaaactggtcatacatatcagcttctgtttcagcagcagtcgtatgccttggcttatcttccatacttttaacatactcgcaaaataactctgcgatagcagcgtctctgctttctgggattgttcttgaaactttcatggtacgactctcctttctcctcttatttccccaaactttcaagcgtctgctgtgcgtttatgatttcgattgtttcagccagtaccagcggcggcacataattctggataatcataaccgctgtattggtctggttgcgtttaatggctttataagtggatatgccgccaaactgccgccttaactccctgtgtaagtcggagtagagccgccctctaaggcttttgtcctgataagcgttactctgcttgccgcccagacactccacaccacgtttctttacagcagatgtgataaggctttcttccacgccgaggattggcatatcgttcttgaactgctccaagtctgctttgacggaatcaatctttgcttccagttcaacgtgaccttgtgccaaaagctgaatttgcccttgtatggtggtcgggaattgcggcgttctgttctgtcggtcaaaataattatcttccaaatagtcaaattgctcccatgccttgtctgtgcctaaaatcttgcaatggcggcttgctccccgacgtgtccagaggtaagcaatagatgttgtggtaggcagtccgctttcagcggacaccctcataaactcttttaattcttcaccttgcaatacaaaatagtgttttccctctgtaaatctgtccttattccttgaaaaattattagaaacatttttagtagtagttccatagacttctgcaagctgtgatgtggtaataaccacttgaccgccccactcaacgagcggttgactggaaatgtctttgattttgtgcataaaataaaaactcctttcaaattgttcttgaaagaagcttcccattctgatatgatagatttatcagatggaaaacttctgatatcaataatagaagttgcaaactttggttggggagcaacttctattttttattcagttcattcttaatcagaccaattcctttcattatagtgtctgttctggaaagctgtaattgttttgcgcagtcttcaatatcctgcttttctgcctttgtgagacgaatattaagcttttctgtacgtgatacatcttgtaaaggtgggcgtcctgtgcgtgaagacatttgtttccctcccttcttttgtcctcgcataatttattatatattatgggcgtacataagtcaagcactattttcaaaaatttcctgtccatgattttaaggtagctggctaattcgccgagggatatatcagaggggttgataattgttagttcgtcctccatctcatgaaaacggttaatgtaccgcgcggtgaactctgtccccttctgtcctgtcagtttgtgggcaatgaactcgcagcccttctttgtgacaaggtagcagggcattgtcttattctgttctgtgatataggttgattctgtgaagaaatcggagagtccaattttggcttctcctaattgggacacataattccgaatatctctcaataatttagaatggtctttaccaaccatctgcgatacctcaatagatgtaagaacctgtccgattttagacataatgatacctcctatgaaaaatatgttgattttcccataagaggctgatataataatacttgtcaatctcttgtgggattggtgccttgagtagtcgtatcgggtcgtcaaactggaaacggctactcttttaattttcttattcctcgtctgatagcttctgctttttcgactttctctttctcacaatactctttcagtatttctgagtgttccctatcaagccttacagttacctgttcagatttaggattgtcagactttggtctgcccatttgtgacatattcttttcacctcactttttgaatgtcactaagtatagtatagtttatgaatgtcaaaaagtcaatacccaaaataaaaaaaacaacagattttttctactgtccttttctctttaagtaacactctatcatttcctttcaatgcccatacaaaatcaatatatttttgtcaatagttttacttacggctttattttattggcacttaatgatatactcataattaaaacactaggaggtataccatgagtatttttgatttcttccataaaaagcagactattccacaccatacttgtgaaaccattaaacgacaaatacagattttaacagaaagcataaatcttgtgaacgattcctgcgatgtccaaactgttttgaaacaatatgatatcacctgccagacattagaacatctgtcaaaatatacagatgaagaaatgcaggcagcgggttttaaaactctcggttcattatcagaatccctttctaatatattagctgataaggaaaacattatcaatcaagccatacaacgaaactttgacaaggaaattgattcacttactacaactgatggaaaagcaaaggctcttgacactttgtataactccatacgcatcatggatggactagatagtgtaaacatttcttttttagatgccctatatcaggagcgaaaaaacaatctttgtataacaatacaatttggacagttcacgcctctaccaagtaaacagttcaaaacacaaactggtctcgacattgaatcagaagaatatgttcaattgtcaacatctggtgacgaaaatgtatgtcccatgtgcgcacaatttgaaggaaaaatattccctgttaatgatgccccgaaacttcccttgtgcccaagttgtgcatgtgcctatatgcactactataaaaatgatttaccaagtaatgtagtaatcagtcataagaatgattttattttgccatcaaaacatacccctttattttataagcatcagcagatcatttataatgaacctgacattaacaaacaaatacgcttatgtgaaaacgacttaaataaattatctgaatttatgacaccatatattaatgctaattttaatgccccacaagaattggtttgcagagatttgcttcctgaactctatatgcggcttggaaaatgggacaaggcagaaaaagcaatcaagcgatgcattgaagctaatgcctattatcctgataatggttctgaggaacttctttatttggctacttttcgcaaagtcgccacacaggctatttcttacataacacagaatcctggatgtcttcagcgaaacatttataaagcattaccctatgaaacagacgaaaaagaatgtttaaaacattttcttcgaaacagcctattaatcaccaaagaaaaatttaataatacaaacaaactgtatatcccaaaagcataaacctatgtttgtgctttcggtacttcattttatcgtaataattcttctttgtcattatttacaagatttaaaagagccacttgcaaaacaagtagctctaattttctatagcctcaatacttctccagcacagctagtaaaattggtacttgcactaaattgcctgatatgctatcaagtacatatgttccattttctcttttcaatacaaattcaatcaattcttctccattagtctgttgaaactttaatcctattttattatatttgatttgatcaacgtatacaccaagtccgctcattgaatttaaacattgtttatatactgcctgtacaccctccgaagtagccgaataaggttcctgggaaacaagttgcaaatatttttccccatctataagcactaaacaatcctctccaaaatatgttgctgcccatgccctagcaaatgaatgttttctatcttcatctaatttttgtgttttttcatctagcaattcttttttcgttttttcataatcctgtttcaacgtttccaattgttgacgtgaagcttccaattcttcctccagttcatttttttctgtttggagcttatcataatccttttgtgatacaccacaccctacacatgttccaaaaatgatacagcacataattactataatttttatttttctcatactcatatcctccgtttttgggatattataacatgtaatgtaaaaaaatggaataatatagtacaaaaaaagaaatctgctgcaacagatttcctcaatatgatttagaattttacaaatactattttgccacctttataatcaaaaatgaacatttatattatctgttataattgttcgcacttacttccaagacatctgcaacttcttcttattctttgcacaatccgataaatacagattgataagcgtctggtatggtatgcccgattcctcgctctgcactttgaaaaaatccactgtatcgttatcaatatttatcgttatctgctttttcaacctttttacatatgggttcttcctcggattcaaactttcaatatcatactcctctttcattgatttcacctcctacatatctgttatatgtttttatttcacttttggttgctttccttgctgaaatgattctgatgatgttatcatcgccacggtagcaatgactcacaatgcacaggttttcatgtttggtaattccaagtattaggaaccgctcttcctccatagagtgttctgggtcgtcaaacatgattgcatcatcatcataaaataccgttgccgcttcttcaaaatcaattccatgcttagctttattgattttgtttttattttcgtcccattcaaaacttatatattccataattgtattataattatttagtaattatttgtcaatacccaaattaaaaaagacagcagttttttctactgtccttcttttcttaggaagctggctaactcacttaatgagatttcaatgtatctgttgtatcatttattcagttgtcacaaaattcagtataatcaatttgaacttttaccatattctaataacgccgccaatggacgacttcacaccgatgcgtccaaactccgaacaatccggcattttcttattaatcacatatgccaatgcctgcccttcttccaagtcctcacgatctaactcccactcggaattatcccgcttcacatacaaaaggctatatagatcgccaatgttcgtaagctggtggataacatgataaacaaccacctcatgttccttctcgaactcatttaccattgcctgttcctcatcatctagccaatacaaaattcccatatgttccgacttattcagcttttcttctcccttaaactcattcagcacattcttatgcaccttcaaaacttccagtctctttactgcctctgctcttgtttcctcaatatttcttactgccatatacatttcctcctgttttaagttgttctactgatatttaacttatacgcatcacaaatggaaaattcgagtttataaatagttttgctaaaatttggaattttctatttttccccctatcttgctacttttcgacaatcgcccgattgatacaaccatctgaccgcctactgcttcgcttagtttaggtatttgtttagcgtgaacgcatcgtaacactacctgctggagtgtcaatagttttatagaaaatattttttggaaatttcgagttttttcacagatttatccactatatccacaaatatatatgtctttatatacgattacgaccataatgacagtacgagcaataactttccagcaaattattctatgaacttttcttgtgactacctaccggattgtcctgtaaacatatttgcacacaataaaaaaagacttattcttaaatttgatgaacaggtcttttcttaaaaatattgtgaaaaatccgcgtttaaatgacttttataattatggtcacacttgtttaaatgtctgacgtgtatttttacctacaataccatctaccgtaagtcctaacctgcgctgtgcttctcggattgccatatccgatttagaaccaatgacaccatcaatttcagatttgtccagcaatccaaaacgccacagacaccactgtacccattttgcaccttctccttttgtgcctttccttatattcgttgttggttctttatatgggttactttctggtgttatcgctacatttgttggcgctgttttcacaccaagccgttcattcacttgttctgcaatataactatgcagattatacaggtagttccctggacaggctttcgctgcaaaatctctatgtacagtcatattgcaatcatgcaaatgatttattctatctgttttatttgtgctccatactaactttttaatagaatttctcttacaaatatctgttacaagttcaatcaagcttgctatagccatattggatacatgccaatcgggggctccaccatcatttgcaacttcaatagtaactgctctatggtcatttttcgcattcgacgatgcccatgacctgtctttttcctcaacatacaatgcaattcgtccatctgttcctatcccataattagaagatgcttttctggaagacgaagcaaatacatttccacatgtttcaatagatagattccctgccatacagtgaattgtaatagtgtcaattttatgattccttggagatgtcttattgggacttatttttctataaactaccagcggactattggtaaatctcatttcttttttcctcactttcttatatctgtctaatctcgttcaaaaaaatagggtggcaaattgccaccctgaactatgtttcattataataatttcaaatctactcctaaacggtctttcctttcttcagaaaaaccgcttacaaaatctttgacagccttatttctctctaacattatcctcatttcctcaagagcatcatcaaccatcatacttactatttcaaagctaattgtatttctaagccagggaaactttgtgataaacatatcgtaaacatatcttagcttcagtttgccagtgccagaacctaattctttttcagccttcgtcacagcccatagcatccattcctttaaggactttagttgttcagtacttgactttttaataaagatatacacactatatccagctcctgccaaaatagcggtaactgcaaccatcacataccagttgttaatgaaaaaatccataacttcctcctattctgacatgttttcttcaaaactattgtcactctgcttttccatgtcttcttggtacttcctatcttctaattcatactgtcgttctttctgtcgctcttttgttgtcttaatccaccccatgatgccgcattcccctccacacactgtaaacacacaagtgcataaggtatctgggattgcaccagtgatgtaatataaaaccagcattataacaataaaagcaagcagaaatacagcaataaatattagaataatgtccattgttcttttctgtttcttcactctacctgcctcctataatccaatttttgcaaaaacaatggcgagaacagcaccaagtatggcagtaccaatatatgccattgccttacgccatttctcgccgtctctattttcaatttttgccagacggttgctttgttgttcttgaactttcagcatgttttccatattatcagccagcttttgtgtagctgctgttaagttggaaatctccttaatactttcttccagattattaatgcgatgattctgtcggtcattttcttctttgagtcggtcattttccgactccatcagctccttaaattgctctaattctctgtgtgttactggattatccattcctttttctcctttcttccttttttgcgcataaataaacgcatatcaacaccaaatttctcttttggtgttgatatgcggtcatttagggcaataaatagtaatttagcaggggtatattacttaggtactggcaccagttttgatataaagtctaaatttcctgacagttatcaaaactttacagtagataattttattgctggcggactttcgggcaacgttgtagccgccaagtcttatacagatggaataagtggtgtacctgtagcacgcgccaatggttttagtattagtaaatcttataatgcccagacaggcgtattagaaataagcggcaatacacagcaaattaggcaaggatatgataaggctgttaatataacatacggaaatgcagcacaaacgatacagacaagctgttggttagttataggagagattcagcaaccttaattttaactaaagactataagaaccctaaaaccttatttatacactttcgatatttcctataataacataaaccttattttctgaagtatacgcaatgccatgcccgctggcactataagaattgtaaaatgtaaggattccagtactattattgtactctgtataccaattaccagaaccactatgctgttctgccgctccttgtgaccaccctatcttgccaggcataacaatgaaattatccgctgttaaattggagtaaaggttaggtagtttttgagccacatttacagttttttcagatctaaggtagctgccaagataatatacttcattgtgttctaaattactatttttcgtcctaagcttactcgctgtgttataatatcctgccactggcactgtcagatatgtataggtgtcgtcttgcgtggctgtagcatcttgtgttgtactccctttgtttgccattgttccactggtctctacattactgctattcgtaaaagtctttccagacagcacatctcctgcccctgctgttccctttttagtcgcgcttacacccgcattatatccacccttatagtttgcactgtctgcatttactcgcccatctgcggcgctgactcccgcattatacccacctttatagttggcactgtttgcattcactcgggcatctgcatctgtaacacctttattgtatgcgcctgcaccagatacatgtccctgaccattatggtagcctgctggaattgttacattgccactccctgtagtcgctcctgtccacgctcctttgtttggcattgttccgctggctcctacactactgccatttgtaaaggtctttccagacagcacatctcctgctcctgctgttccctttttggtcgcgcttacccctgcattatagccgcccctatagttggcactgtctgcgtttactcgcccatctgccgcgcttactcctgcattataggcacccgctccagatacatgtccctgaccattatgatatcctgctgggattaccacgtttccattccctgtagttgctccggtccatgctcctttattgggcattgtacctataattaagctgccttttaccccagctttttttccagctaaaatgtctcctgctcctgcatctgccccgctggtatccatattattgttactgcctgttattcctaatattgtattcccctttacaagttttgctgctgtaagaccaattaggtttgctattgtggtgtatgcagcatacagatagtttcctacaccgtatttccctgttgctggtacttttaactttatacgcttgtttgttccatctatagatgcctctgcgttatagtctgctgtgcctgtcttatctgcaagggtgcctgtaatctttgttccagcagcgttatgtgccgtatatccactcaaaagggtttctgctgtaactgtatcttttgatggatctattaaatctagcacttttcctaatagctgttcccaagtttcatttgcagatgatgttattcccaaggcagtaagattttccgcaagctgttttttcttctcactgccaagctgaaaaacctcgtttattgcaccgataacatcttctgactgaatgccttctgcaaattctggtccctctgtattgtcatcactcccaaggtacttgtccattctcttttttaataccaaatcattctgaagtaactgcttttctgatttggtcttattctcatagttcacaaggtctgcccgctctacaataagaatcttatcagaaaatactggattatcacttgtataatatttcataacatttccccctttttaaaagatatcttctagcacataggtttgttccacatcatcatctttacctttgcgggtaaagtttttaatgcagataatatcgccgttctcatcatacaacccaatttcgcttatctcttcccctgcaagttcattgcctatgagcgtgcactcgtaacggcatatcgtatcttctggaaaagtatagccatcaattggttttctgtataattcatttttcaaagctgcctgactatctggtggttctatcaccgttccatctgtttctacacctccattcccaaatgccattccgacaactttcggaagggttattgctcctgctctagcctttactagcttcattcttgctgctttcgttattactacatttttgtttgtttcactcatct

SEQ ID NO: 2 - protein, 450 aa, Unidentified
  REGION 1..450: note = Bacteriophage strain LSP1
MKKEKFDLEHFPTSGSAQKMLSYVSDGFYDKSYIGKWLYQVMGLEYDKVLDIIEELPVQFFPETATWGLMYHEIKWGLPVRLNLSDEERRSLIYQKRDCRAPMTPYRMEKYLENVTNFEVHIADANDLGVYGFVPSHPNVFKAYFLGEGTLNSKLVHDVLNKLKQSHTIYIINDRIEIGFNNVHLEQLRVACIFILYIPFWGDDIYTGPYCYDGTLLYNAVRKYKIGLLIRYLMHTQTQQHVAYKYNRIKIFIQGREYTNLSLCSFYSISFWGCSVYNGQACYDGTLLYNAVRNYKVCIIVRNYIGILTLQRIVCSRNCTRVLVQNRMQTDLLLYFPYRIRQKEFILKSIVQHLIKLNVVEEINHNSQILSMQVLHKNILNIWHRYKGKVKNNRPRVVDKIKIQMNINSPCGQLGNMQIITQRNVAYYDGTLRYDGTAKYDALYKEERVE

SEQ ID NO: 3 - protein, 372 aa, Unidentified
  REGION 1..372: note = Bacteriophage strain LSP1
MQPEFIRPDFIENNSADEIHQRMMNNLPADIDNMPGGFPYDFTRPAALEKDEFINYHLVRALMIAFPQYAWDNWLDLHGQQVHLERHPPKCASGKVKVTGIPGTIIAEGTIFCTPATDSGPSILFYSTEEKEIEAEGTILIPVLAVESGASSNVPANTVTLMAKPDKNITEVINPEQITGGTERESNDNFYDRIATEYDNSLTFLGNDSDYIRWAKESGAGDCIVIPTAEGPGTVKIVLVDANGQPANDELVHKVYNYIVSPEDRNKRLLPTACAKLICGPATTVKIDYVITGLFYDETTTIKQIEKDFSTAVKAVYTVAKQQNILRYNDVRPIISNIAGVIDFDEFLMNGERENIQLNKEEYPKTGNFQFS

SEQ ID NO: 4 - protein, 145 aa, Unidentified
  REGION 1..145: note = Bacteriophage strain LSP1
MSQPLFPVVQVPEFIPQNTRYDMEYRRSAKWDPVTNDFVRDGANRIVECDGKEAYAIWCFKIAQTERYRCLAYPGSIGVEMERALDNDDVETVESMVQRTITEALMVNPRTEDVLDFEFSWEGDSMHCKFKVKGVDCDKEITITI

SEQ ID NO: 5 - protein, 127 aa, Unidentified
  REGION 1..127: note = Bacteriophage strain LSP1
MEGFDGHPGTAKLATVLSERMRKETESPFVLDFGEIQGNYSLVTNTFPVPIPKGDYSVCRSINGYPLGTSESSWIGHIQGDKHIHDNPSGFWGHSHDVLLPELKAGDRVLVAWVQSEAVVIDVIEQS

SEQ ID NO: 6 - protein, 415 aa, Unidentified
  REGION 1..415: note = Bacteriophage strain LSP1
MIDLANIKYYAVVMDASGNQYNIGDFIQNLGWEENENEISMRLFFTVRNYETAKGYLSSLIKPGCLIGIFATDGVVSEEVARGYVENWNQVEKNSENSLKCTCYDELYKLQKSQDNRYYPSGTGTKSAIEEIFNDWEILQGDYKGPNASHGKTVCNNKYLSDIILEFLDDAVKKGEKKCMIQAAKGYTSVIPWGSNKTVYVFCVENTQSFSKNISTENLITRVKVVGQADKQGKHSVEATLNGAVQYGIRQRIYTRGKEETLSDANLAAQKILEDEGKIEKKMAVQAPDVPFIRKGDLVYIISSITSSYYYVKSIQHNAESYNMSMDLEYVESEEISGDSDNGQEKKAYQVGDIVNFHGGTHYVSSYSDAKGYPASAGKAKITIKNGSGKAHPWHLIHTDSGSNVYGWVDDGTFD

SEQ ID NO: 7 - protein, 226 aa, Unidentified
  REGION 1..226: note = Bacteriophage strain LSP1
MDIKLIPVGSGAKFTFPALPEKIQGKYGAKYQSFDIISQGTVKIPKGTSISEFSWDGVFFGKSKKMEPIVRQNSWKEPKECVKILSNFIKNETVLNLIVTGTWINMDVTISSFQARPIGAYGNVEYSITFVQKKPLEIYTTEELGIVKTAPRNDSGDSSKGGSTYTVVSGDTLWGIAAKKLGSGSKWTVIYDANADTIEAEAKKHKKSSSDHGHWIWPGEVLTIPG

SEQ ID NO: 8 - protein, 1561 aa, Unidentified
  REGION 1..1561: note = Bacteriophage strain LSP1
VRTIAETLRIEIPIETVDKTEPEISNLIRKLGNLETAAEKTGTSSQKAGERVSQFDKQAQKTEKGLAKWAKEKYEILLEAKDKIAPILSTIGSGLKNFAGKTWNVTMRAVDLITSPVRGIINLLKNPVFQAGAVLGVSIGLKDTIDTYKGFEAAMSQVKAISGATDSEMTKLTNKAKEMGATTKFTASESAEAFNYMAMAGWKTEDMMGGIEGILSLAAASGESLGTTSDIVTDALTAFGMKASEAGHFADVMAVAASNSNTNVSLMGETFKYAGAMAGTLKYSIEDVALATGLMANAGIKGNMSGTALNSIFTRLSTNTHGATDALKKLGIAYFNSDGSARAFGDIMKELRDATADFTDEQKANLANTVAGTYAQKGFLAILNATTEDYEKLSKAVNNADGAAAKMAEIMMDNLQGAITLLQSAADGVKISFGGRMAPYIRSFAEWLTEQMPMIESALDELMNWVDTKVDQMQRKFDKLTKTKKWKDANFFGKVKIAWDEFIAEPFLEWWNGTGKEKFAQNIGNGIGTGLRTGIMTLLGMNISEISDEGVSFGAAFAKGFSEGFDFDTVSGKLWQGFKGMFSSAGKLLPGGESAGLSSVLSAIVLSKVAKLFIGMAKGGVSIGKGLFGANAATGTSLMGSFLGSAAAGTGLLGKSGLLAINLGAGNLAGGASMSAGALSATGMAAGGGAIAAGVTLISSALDAYKAIKSDNKEESKAYGESAAWKAGGVVTGAVTGAAIGSFIPGIGTAVGALVGAGVGGITGWIKGNKIKEEYQEEYQKNVEEMQKEAQKAQKVFEATGFAIEDVTFKNKALVQAMNDSEVSASQFALMFQEECANVAKKAFGDISLSLAEVKKVASEITFGNMAEGLNMFAQVTSDAEAALNSLELSVSDLKKENWKVGLNMELSETDKDSYKGAIENFLNASQTFIDDNHYQATVALKLLTGEEADTSSLDSYYEGVKNRVNDLSAELTDTMNAALEDGIIIPDEAKALEELQSQIAEITGKIAKAKTDAEFQALQIKHKGAALDADSFSAVQVEAQEQKSAGEKQYYNALVETITNLNLQYTYGTITDAEYQDKYEQAMAGYQANIEELDVNVRTFNLDSIATAFEEQLSDFMPEMEGSLSEKLSEVLNNTIIEHPNITAWTDTDIIKWIGLDKLNIDSADQATIALDLKQTALAVPEETKRQLIANYLKQVPTAEEIRDAVDWSSLTNNDVSNMLQQVGVVIPDNGIKQVDMPLEEWARQFGKDYDELIDDYAEQMSNSLVDNKSVENLSAFTKQYMPNFSASDDARNIKDIMAQYGPVSNERYDELIKEWEDTGVDFGTALSHGASATLLDSLPLLRTDMQTALEAAIADPFFINPSINMTPTYNITTPTLPTVNQETDGHAAGGYVSGGPQLSWLAEEGYGEFVIPTNPSRRSRALELYKQAGDALGVSAHASGGYVGGSISHNTASSYNLFNDANKYASTAYNETTQDNYNEETVQVYEPVSAERQSSLSSSPIQVNVSLAPEFVIHGGDGQSEENIMQIIRRHMKELADELNGEIAATLEDVFSNMPLKEA

SEQ ID NO: 9 - protein, 16 aa, Unidentified
  REGION 1..16: note = Bacteriophage strain LSP1
TGTCTTTCTGCTGATA

SEQ ID NO: 10 - protein, 49 aa, Unidentified
  REGION 1..49: note = Bacteriophage strain LSP1
LLHHIFQTTGIPPDEFYQKSKGVQAFMLASMRITLDSRTKEGEDNSRDT

SEQ ID NO: 11 - protein, 197 aa, Unidentified
  REGION 1..197: note = Bacteriophage strain LSP1
MSKDFRKGNFTKGVTMGESDTTNEATQKVEEQEITEYETNEEGTKILIRANEEDFIQGLIDAAEYASEETQRIEIVREGRLYFAFHVRPLSSQEYEKCKKKYTKYVRNKQLGMKLPEDTDRIKYQSAIIYEATVEEDRKNLWDNHKVWNALNAKEDRIMNGLDVIEYSLKAGEKDKVLEAIDKLSGYDDNLEEVAKN

SEQ ID NO: 12 - protein, 154 aa, Unidentified
  REGION 1..154: note = Bacteriophage strain LSP1
MRNERAAGDSRHGRTGKDGAFYNKDGVLLATVEQFTSNVSWNNAKYSVLGDAQEHETANTFSVSLTMSQVVVEDDAFIEELMEALETQVMPVWDFQGSLLGRNGSEERVIYRECIPSGQVDIQNVTTGDVIKRNWNFFVNRAPKLQSLLGIDRD

SEQ ID NO: 13 - protein, 469 aa, Unidentified
  REGION 1..469: note = Bacteriophage strain LSP1
MAGTFILGETKVRPGSYFNIQKKGNNAVAGIMNGVTAVIFKADFGPLNTAVELNAEDGYEKIFGTGLTTDAMREAIAGGAKTIIACRVGNGGTQGTITLKDVNDSDTLCITAKYPGEKNFMVTVREKLSDSSLKECIFYAGTTEFEKVEFASGEGEAKALADALAVSKNFKAELKQNKERAVLEMISQRAFTSGTNPETTTGDYANAFAQIEPYEFNTVCLDTEDTEIHLLLQSFVNRIFDAGSLAQAVVAEKHTVDLETRMKHAAALNDEKMNYVLNAWIEEQGTVIDGYQTAARIAGMIGAVSSGSSLTHTVITGFSEILEKLTNTEMISAEKKGCIVLSYNKVKQVWIDNAINTLITPADNQDDGWKKIRRVKTRFELIRRINTTTDDLVGKVDNDNNGRATVISQVQGVGDSMREEGKLVACAVTESSAYKADGDSAWFDIDVIDKDSMEHIYLTFLFRFSTNEE

SEQ ID NO: 14 - protein, 89 aa, Unidentified
  REGION 1..89: note = Bacteriophage strain LSP1
MAKETLKETKTVETTDIKVEVTQEQAPKKEQRTLQESIYSVSELAANARKVFGVRQECIEAALKAAGKTECTVTEAKGIVEKFMKREVQ

SEQ ID NO: 15 - protein, 291 aa, Unidentified
  REGION 1..291: note = Bacteriophage strain LSP1
MILEELIYKRFICSKNLIENLASFGGGPAIFSTEPPEESQEGWGGNTQYPQIVYNFDLLANEERHSAGTLFVSLLCQNTTEVMPEQIEPFIKDCLRDVILKPEGGTPYCFAWARTDAFTIDEKKGNTTIGSEVRFDILEYPSQETSDPDPIMATNKYIKELYPECFIMGYDQMEEITEATGKRPVIYCRLVSVDKSEETNTVAWLDGRIAVHILCPESETRLKMAAVIANRMSLDGEIIMLDHSPMFIKRLQANYKSDYLKDGQIFVTGHYGLLRYKEKPHSLAAAHVRYK

SEQ ID NO: 16 - protein, 111 aa, Unidentified
  REGION 1..111: note = Bacteriophage strain LSP1
MALIMEKPTAAFDMSQIKRGDLLWGKHCTWNEGKAGFVTTAAEQQLIVQYYPGIGNVTNHFVIPVSEVIDGQWEIRWSADMTEIKEYEVETDKEQQETKGVEDNDIRRTDL

SEQ ID NO: 17 - protein, 154 aa, Unidentified
  REGION 1..154: note = Bacteriophage strain LSP1
MKLWADGKNGSAGSAIHATIKEQVSEVNRKVISRGVRAVNAIRNAELEVLKGQRSGKVYRKPFTKHATYTASAPGEAPARRTGNLRMHWNGQVRSENSSNGGVAIVAELESQEYYAGILENGTNDGKIAPRPFVEKIKEKAMPEILKIYSEPYT

SEQ ID NO: 18 - protein, 112 aa, Unidentified
  REGION 1..112: note = Bacteriophage strain LSP1
MYVRPGNLFKDFIVEENKQVVTGTGRVANSHSGDGTKILKGCLAETSDEERTNHSQKDHVVTHTIVQAGSPKAKRTDKLILGERVFYIVDIDDTGMLGISTIYYAEERQDVK

SEQ ID NO: 19 - protein, 136 aa, Unidentified
  REGION 1..136: note = Bacteriophage strain LSP1
MAKGTYTYDPGNVKELGKDRMRFELGDVMVEGCSDTTALTDEEIQAAIETYPKSWKKAKLMLLESLCRRFSYEVSTKTGPLTLELQERAKLWREDYDKLKKEVSAECSVPHFSKQAACKPPYFYTGMQRNERAKSR

SEQ ID NO: 20 - protein, 212 aa, Unidentified
  REGION 1..212: note = Bacteriophage strain LSP1
MRLIANKPCSFGGKKFYIGNEIPENLVADARLQEKMGVITIVNDSMGVADKQSATLFTQEQVDKMLAEAIEEAVNNTITEMEQKQKELQEALQQSAAERKEVDSETLMETVMIDIVIDSDRENEQHMTVLAKSEEIQQVFSIMQLNVDEGAKAIADVKSENVLILLHAADSRKTVKNAAKEQADKLFSIKAASNESTSGNKATGTNTEGVDT

SEQ ID NO: 21 - protein, 342 aa, Unidentified
  REGION 1..342: note = Bacteriophage strain LSP1
MAKRTAASIQADIAKGAFRPHTALSNMALAYYQSDEKSFAKTIFPICPVSLSSDNYYIFDKEDLLRDNWHRKPAYGKVDPAVLSEHMDTYACVVDQMIMGIDQIRQTDLNRRMGPRITDPKQQRTKTMASQANIHQDAWFARSFFKKGVWGQELTGVDSTIPTSGQFIKFSNANSDPVSFMDEKKTAMEESTGRMPNRLALGVNVFNALKKHPAILERVKYGGSTANPASVTLNVLAQLFGIDRITVQRSIMNKAELGQTAKMEYIGDPNAFLLAYATDSPSIDEPSAGYIFTWDMLGNGNILPILSYLGENGTHSEFIEGLMAADMKKTADDLAMFFADAV

SEQ ID NO: 22 - protein, 140 aa, Unidentified
  REGION 1..140: note = Bacteriophage strain LSP1
VGNRNFNGVQVNQSVTIVEQAGAAIEDVRNRIMAYDKDGNVVLAADGSTVLIGVALIEAGINDISGVESGKVNVGDDVDIQIKDIGYILAGGEIAKGNEVTASNGLAVKAESGNYVVGIALSTVAKDDYCRVQIAKYQKA

SEQ ID NO: 23 - protein, 440 aa, Unidentified
  REGION 1..440: note = Bacteriophage strain LSP1
MINQKAFVNRCLFVLYIPQKGDKKVATKLRNLKIKKVDFVDEGANPDADIKMRKKKDREEPKNSSSILKKLFGFIGKAAGMNQEEIDSAVAEIQKGDSISFSEKVNEVNNQKICDEIWDIGYALQSSLCSILNDDELDSTSAAAAMQESLDEFYAVVQESIRKWSSGRSASIVKKNEDISEAELEIMKSAVERLNETIEKAAKEESEKEEPDNNQKPKGEEKEMGMKIDKSKLTDAERVFLENIEKRYGVDEGDNTVDGGASAPLEQLLPPVSTEPTVVKSVTQPVPNVVASVVQTEEPDSIYKGLHPAVRAELESLKKFREDAEARELTEVAKRYVIIGKKEEELVPMLKSLKAAGGTAYNDMIAVLDQAVAAVEKSGVFSEIGKSGHGSTAGAAEAKIETIAKGYMEKNSSLDYAAAVARAWEDHPELMSEYETEAGF

SEQ ID NO: 24 - protein, 165 aa, Unidentified
  REGION 1..165: note = Bacteriophage strain LSP1
LGTGPFFIFIFERGRKDKVKKFSELIEKQNEVLKGRFKITKSDDDRRLAFGWASVSIRADGELIEDWQGDIIAPCELEEAAYEYVRLYGEGGEMHERGGVAILIESVVFTEDKMAAMGIPVGTLPVGWWIGFKVTDSGVWEKVKDGTYTMFSIEGEAERIEIDEA

SEQ ID NO: 25 - protein, 60 aa, Unidentified
  REGION 1..60: note = Bacteriophage strain LSP1
MENMVQRLRTDEELLEMRKEWKELDIEKSFPLFHFETYLDIEDYKKRVRKQLDDYKKQKK

SEQ ID NO: 26 - protein, 62 aa, Unidentified
  REGION 1..62: note = Bacteriophage strain LSP1
MEKSGIMNFLKTDKELQDMRKRWKELEIEEPFPPFNYDEYNGTSGYKEAIRKLLIDYEKASC

SEQ ID NO: 27 - protein, 527 aa, Unidentified
  REGION 1..527: note = Bacteriophage strain LSP1
MGLRIIPPKRVQKVKTANSQEVLERLEEYLENNCEEPVQILCGFWEDQQNAITYQELRQAVLDGTLSKEAIELWRQDYSKLISGRLKGLWTNAIIAGASGQPILDDKTFVFNTQTPGMLKWIQTRGAQLVTACTSEQKEAISILLAKKMKEGHTVDELSRLIRPCIGLTKGDAQAAAKLYDSIIENLKKEHPRMKPENMQRKALDATCKYAEKKHRQRAMTIAQTESAFAYNRGADEGIRQAQQQGYLGTVKKRWSTSGDDRVCSTCQALEGIEIEMDKSFPFKGRMLFPGQDLLPPAHPRCACAVEYIEVENHSELIESPMRTGDEWIDASIDDYDSSLINTNGDEAMSIRLLMSHDEVKYEKNETLNLPFVYNSDRDVILYNPYAENANDYDMRFALTHENAHRVDYLFLHSWENENFRRAMDNTESLIISNLDGLEKLLQNDDYGSDFAFNDLISSLTRGKANGMLFIGHTPEYLASDYKRSLEVFANITCIDMSESAIRKEFDGILKELYEAYKEIIAWKSLV

SEQ ID NO: 28 - protein, 462 aa, Unidentified
  REGION 1..462: note = Bacteriophage strain LSP1
MANNKEIGRIGQRRYGGNIYEEFLYELRGKRGIEVYREMSENDDIVGAILFSIEMLVRQCNWNVQPGGDTAKDKEAAEFVESCMNDMQDTWIDTISEILSFLTYGWSFHEIVYKRRMGNTKDTRTKSKFNDGLIGWKKLPIRAQETLYQWEYDNEDNLLGMTQMPPPDFGTFTIPMEKALLFRTKSRKNNPEGRSILRNAYRSWYFKRRIQEIEGIGIERDLAGLPVLYGPDGVDLWDKNMPENADILAGLESMVRNIRRDEMEGVVLPFGYKLELLSSGGTRQFDTNAIVNRYDTRIAMTVLADFIFLGHDKTGSWALSSDKTELFAVAIGAFLDIICETFNSQGIPSLIDINGQYFSGITEYPKMTHGDIEDADITKVAAFIKDMTGIGVLIPDDGLEDYIRQVGHLPDRTYSDVREIDRTRQEQQEQNQPPEPKTAAGTEPDEEIQDNKVEEAKKRLGR

SEQ ID NO: 29 - protein, 604 aa, Unidentified
  REGION 1..604: note = Bacteriophage strain LSP1
VAQKDLKPVRTKEEAKERGRNGGIKSGEVRRAKKTMRETAKVLMSMEVVGQKNKLNLEAFGIKKGDQNYQTAVVVRLMQKALVEGDTSAIRLIGELTGDLNRFSFIPEEESEIVEITYPAINLPNNGRDKKNVFELAPQAGPQTQFMASSADIIIYGGAAGGGKTYALLLESLRHKDVRGFGAVIFRHNYNQITAEGGLWDASNKIFGQIPDAHSRKSPKLHWRFDGGAKLSFAHIERDEDLKSWQGTEIAYIGFDELTHFTKHQFLYMLSRNRTTCGIRPYVRATCNPDADSWVAEFINWWIDQETGYAIPERSGQVRWMVVLNDVIYWENTPEELAKKYEVNVEDCKSVTFIASYLENNKILMESDPGYLANLKAMTEVDMERLLRGNWKIKAAAGLMFKRTKVNMLEVLPTDVVLWARGWDLAATSEDEDGEPAYTAGVLIGKRKNGRYIVADVINRRLDSAEVRELIKMTCIADKAKYGRVITRLPQDPGQAGKAQAQSFLKFLSGFAVKILPESGDKVTRAEPFSAQWLGLEGMDKGNVDILAADWNEMYFNQLESFPQSQFKDMVDASSSAFSEIENGWTYSAPPSDSNLGRSSYWRK

SEQ ID NO: 30 - protein, 83 aa, Unidentified
  REGION 1..83: note = Bacteriophage strain LSP1
MVVRYGKLVKQSDIVREAVKAGEWKKALRIAKDFRINVTKAQRDTMARAYECIVHPEFYKQIGIDIPRAIEKGIEVVSCLYGE

SEQ ID NO: 31 - protein, 79 aa, Unidentified
  REGION 1..79: note = Bacteriophage strain LSP1
MKVKVKVYDGVKYKEESKKVAEIEYQIEGFEVVTGDRAIEIGLETDENSRDEYNEYLVLDLGNGETAAFCNSHVDIFRL

SEQ ID NO: 32 - protein, 37 aa, Unidentified
  REGION 1..37: note = Bacteriophage strain LSP1
MDKKIRKLTKVIRALVQLALEIGTLIAVIKMIIESIQ

SEQ ID NO: 33 - protein, 219 aa, Unidentified
  REGION 1..219: note = Bacteriophage strain LSP1
MAYNLMTKNGHNMYDMASMLQKAIRRSNPNLAGYAAYELFGNFHTYMWKRLVVVSAEDCYGIMTKEVIALKLADDFCNKGRKGYDKDPLFAAKAITLLCLARKNRDACYVACNFMLPDRILDESEIEHIDITKCHLGVEGIPDWVFDVHTLTGKRNGKTDLDMTIEEQAALEPKQMSLFDDCSWENYYTWARNQGKVGNKEWADFEQFKRGRKLEPDQY

SEQ ID NO: 34 - protein, 240 aa, Unidentified
  REGION 1..240: note = Bacteriophage strain LSP1
MKKEDKLTLGIYVQSYHRYNKILTQDLLEQCVYVVRASEAELYRKAGVENVWAVPDEEVNNAIRTYWWIVDHAPEDIVFIADDDIEDMMYRLDDTTRLNKDKDTIMAEIERIAQMMVDLNVGYACIDATGIPYGYDGEFAFKGTSGSLKWVYKKVLKARPDENCKYNYDLDLVLQELLYNRIILKPRYIICKDYQDVNAGGDSSKLRQDQIDSIENMKRKWGKYFKYNYKNNKPQINVPR

SEQ ID NO: 35 - protein, 236 aa, Unidentified
  REGION 1..236: note = Bacteriophage strain LSP1
MMITIHQPCYLPYLGVFHKIWKADAFVFLDDAQYSNGYVFEWNRIKTPQGECRLKIPLHKKFGQTLLEVTPNDDLKWQYKHIKMVEMNYKKAPYFSELFPAYKEIVNKCYDNLAQLNIALMNFFLFWFGWNEKKVYYTSNWKLESRAEARVIEICKRLGADVYLSGTGGRNYQKDVHFQENGIKLVYQEWESMEYQQLWGDFLPYMSILDYAMNEGHDIDSHFKKMEKVIADEKRR

SEQ ID NO: 36 - protein, 222 aa, Unidentified
  REGION 1..222: note = Bacteriophage strain LSP1
MKYLVVAAHPDDEVLGAGATMHKAVTNGDEVYVCLLSHWSPTRDDNLEDGIASSHAILGVKKSYIGDFGCMRFKDEDHHAIVRFIESAIKDCQPDILITHHPADVHVDHGITSECCIEASKLPMRQIVNIQPIRKIMYMEVPSSTDWNVSTTNGFFTPNIFVGISEKDLSEKVKAVGVYKDVIRKAPHPRSREVINAMAVLRGSQSGLLMAESFQMVFGLGV

SEQ ID NO: 37 - protein, 177 aa, Unidentified
  REGION 1..177: note = Bacteriophage strain LSP1
MDLFGDFGIFLVDEHDNVIAGNQRSVILARRDPDIEVDVKRLIGYSEAELRSINIMDNTHAGEWDLELLADWTADLNLDLGIDLNNESPEERKIEDMELIRYEKYNYVMIVCKSEIDYNDLIRKLGIEGRKVAITKKRKIKARAIWYDQMKAQIVEKLEKVESPLENSKKDEGEKQA

SEQ ID NO: 38 - protein, 131 aa, Unidentified
  REGION 1..131: note = Bacteriophage strain LSP1
MPKSPNKSIDRSSSSVFFFEIFLGLPKLCFRSPTFILHTSILFIVYYALLLCYSSSFFPIAKKTVHFKTVLLVNWYTTQQNAQGNYPWALFLCFNKEKLYGFVSVVTFDWLIPLHHILFLNATYSFSTCNL

SEQ ID NO: 39 - protein, 191 aa, Unidentified
  REGION 1..191: note = Bacteriophage strain LSP1
METKNEIHMYVLTQEQINQIAAIAGKEAVQTFRAEQAKVEKKRAREENKVNKTKKMLSSYRRIKATLSDEARFTEEEQIDLRWKFIQDLMGNARETVSKSERIIQNGEKRRQEDLYYVYRIEKAIELYHEECEKSGNKEAKRRYRELSMMYLEDREYSVQEISTVENISEKTVYKDLGIACGILAVYLLGI

SEQ ID NO: 40 - protein, 128 aa, Unidentified
  REGION 1..128: note = Bacteriophage strain LSP1
MSKWIRELSPKDKKYGDGWCGDLDRCYRLDRKYIVMTRLIKTNIGIVEHFCIRNRDNTDISWSEKQKIKNDMAGRKRTAIEVFPSEERLIDEAGMYHLWVLPEDYELPFGLHKNDCKTEPIERELMLK

SEQ ID NO: 41 - protein, 59 aa, Unidentified
  REGION 1..59: note = Bacteriophage strain LSP1
VTVETAKNYLRFMERKRMKNEKYNRRFYDDARELSALVSLAEFGNKDISFKAINVIKKI

SEQ ID NO: 42 - protein, 67 aa, Unidentified
  REGION 1..67: note = Bacteriophage strain LSP1
MKKHESRLEREKKTSHRMGFVKGIECTKEIITNELKDAIQNGEIIIAKGSGRLFAIINSVGVLRTED

SEQ ID NO: 43 - protein, 118 aa, Unidentified
  REGION 1..118: note = Bacteriophage strain LSP1
MSKKTVVFDFDGVIHSYTSGWKGADVIPDPVVEGIQSTINSLRQEDYEVIIVSTRCACPEGMSAVKKYLADNNIVVDAVMKEKPPAICYIDDRAICFDGHPETLLEKVRNFTPWNKAL

SEQ ID NO: 44 - protein, 232 aa, Unidentified
  REGION 1..232: note = Bacteriophage strain LSP1
LIDVEATKREKARQLRYKKPIVRNLNLDSIKEELLDIQGKCEDVRWYFEDEDDTLINALDGDEDEAYEFKMMFGDLCSECEQLRWDLEEEFVPECFDQFFVAVGAGEDYGGLLGHDSYEGDYFRLVCSDTAAEDQSRKILKQYTKDNLIEAAMRCFRVYQSFIALTYRYDCLKASMDILRDQNTCYLQMVKQIEELYNKAEKDTIEFKYPFGKEMDKLDLFLNKLPQEAWIQ

SEQ ID NO: 45 - protein, 55 aa, Unidentified
  REGION 1..55: note = Bacteriophage strain LSP1
MRKEIKSSEIKEIILNPAYVINEDFANLKIQGFPVRWMREDEGSDKDIFTLIVDE

SEQ ID NO: 46 - protein, 310 aa, Unidentified
  REGION 1..310: note = Bacteriophage strain LSP1
LIDVDSHNFPNLPLMKLSAWHKQQGDSVEWYIPTQHGFPFPPMDKIYMSKVFSFTPDYEYFVNADEVIKGGSGYCIKLVDGKEVYHKENDSQLSYEIEHIYPDYSLYSELTKDTAYGFMSRGCPRGCDFCHVKAKEGHKAYKVADLSEFWRGQKNIVLLDPNPIACKDWEDILGQLIDSKSWVDFSQGVDIRLMTEEKAEMIKRIKTKNIHFAWDRYGDKEKVIPKFKEFKRVTQWDKRKLTVYVLTNFDTTIEQDLEHIYILRDLGYWPYVMIYDKEHTKSTDIVRKMQRWVNMRAVFEKTKRFEDYKY

SEQ ID NO: 47 - protein, 159 aa, Unidentified
  REGION 1..159: note = Bacteriophage strain LSP1
MKALTIWQPWAGAVAAGIKKNETRSWSTKYRGQIAIHSAMKAIHHTWSNLYMSDEAREVIIRRMKLPEIIDGPVTFPMGYVLAIADLVDCIQITSEYITTLTEDELALGDYTLGRYAWKLENVKLLPKPIPTKGRQGFWNWEPDKELKKDLGVKILELD

SEQ ID NO: 48 - protein, 196 aa, Unidentified
  REGION 1..196: note = Bacteriophage strain LSP1
MSIGEQVKELRYKADIFEKSGCAVDGIVKAFREAADTIEALSAKLAATTPKRPLEYEEHEDYDEGICPICGEPVCHVYAYCPGCGQHLKWGSGTEKSEVVNMEQSVEDCSGWIYCGDGKNLPEEHDSMFAKLKGTEKWDSAMFEKTSDDVNVTVEFENGERKTKTLHTVDGKWNGGQRGVKFKVIAWQPLPEPYRQ

SEQ ID NO: 49 - protein, 68 aa, Unidentified
  REGION 1..68: note = Bacteriophage strain LSP1
MKVAELIRQLEPYRDFDIEARVHLEVMEEELQKRTYKFPCDTYNAELSIDDIGHSDKVVLIGVEIMED

SEQ ID NO: 50 - protein, 106 aa, Unidentified
  REGION 1..106: note = Bacteriophage strain LSP1
MKEHIKAFNMPFEAWDKDIVLGKPHIYVGMYCESGGCIGEFKIVWDNVGIQLQAYDDSWEVLSHMPELIDLMGRIQIEGLKPTIEEFAEMLKGIGFKDRTERERSV

SEQ ID NO: 51 - protein, 182 aa, Unidentified
  REGION 1..182: note = Bacteriophage strain LSP1
MSEVLNGWIEKIRVSSAEIVVHGTKKKPYYEIKYRDLSDGQIHVGYSSYNLDFLFEWLEKCFEVVEKDKASLNEQIRELRDIYEYGAPHKEKDRVIKEAADTIEFLSAKLADMEQSRKDYSEEWIPCKRRMPEVGRPVLVCDKQGNVCVRAVTSNINGVKNWSQNKADVIAWRELLEPYHEP

SEQ ID NO: 52 - protein, 151 aa, Unidentified
  REGION 1..151: note = Bacteriophage strain LSP1
VEGKGDMERLTYKTNGKYTNTTAEQAPPYAIDCDIDTAITKLAEYEDLEEQGKILKLSCTEVYESSGDYVYYIFEDEIVECINCGVQIEADGNIVFTLDANEKIFPYRKPDSENDLSPEDWCEETITLSVCEWGKTVFLIREEAALKLKKG

SEQ ID NO: 53 - protein, 76 aa, Unidentified
  REGION 1..76: note = Bacteriophage strain LSP1
MENTGQSAGRLLIGEILHNLPSITRRNLLYGRKRKVKVKMSCTAYVIVDEDVQGNMEIEDVEDIEDIDDFEVIQRD

SEQ ID NO: 54 - protein, 48 aa, Unidentified
  REGION 1..48: note = Bacteriophage strain LSP1
MKSMRSCKGKYWSRWKEVRKYLIVTLALIAEKCCKTEWRILVKVRADY

SEQ ID NO: 55 - protein, 258 aa, Unidentified
  REGION 1..258: note = Bacteriophage strain LSP1
VKFIASCSYGKDNLKTIDVVLNVLKLPLDEIVTVDVMFNNTISAYYPEVEEFRQKADEIIKNRYGFEIKHLRADLTYENRFYQVRGERAKKENRGKIYGFPIVRGAWCNSDLKMSAIEKYKTQLGESFWYIGYATDEKKTERQEKIINCTNLNMYPLVKAGLTEKDCYEWCKKNDLLSPIYESFSRDGCWFCHYQTLNQLRDLRKNHPDKWEVMLRLDRDSPKTFRPDGTTIHDLEARFASEDSQMTLEDFIKIGGEE

SEQ ID NO: 56 - protein, 41 aa, Unidentified
  REGION 1..41: note = Bacteriophage strain LSP1
LKRLKRQIEGNKLVTQSCARLYFGIMKKMNEAYSEMFDGKE

SEQ ID NO: 57 - protein, 103 aa, Unidentified
  REGION 1..103: note = Bacteriophage strain LSP1
MAFQKERMSSYVDRGNKLIADGKTPEAMKLVEKGLQHYSEKIISAISPYAKADAGLIVLVLRHLADEVERGNVGAKELYEGMKKCINAPSLTEIEKVKKANRR

SEQ ID NO: 58 - protein, 215 aa, Unidentified
  REGION 1..215: note = Bacteriophage strain LSP1
MTKTEVKKVIAVMMATYPNYKPINVDIVVEVWTDMLGRYTYEQVNMALKAYILSDTSGFAPSIGQIVEKLQLFIGNEELNEMAAWGLVLKAMRNSIYHSEEEFAKLPPVVQKAVVSPGQLREWAMAEDVDGTWMNVTQSNFMRTYRAELSQEKTLEKLPPDILKLTGNSNEQTPVIQTNQETLSVSEERKIAEQSASPIPERLKERFAKLMGRAV

SEQ ID NO: 59 - protein, 306 aa, Unidentified
  REGION 1..306: note = Bacteriophage strain LSP1
MHICKKKRGEKSQMAIYRNVHLSFWTDNKVADDFTPEDKYFYLYLFTNPQTNLCGCYEVSLKQIANQTGYNRDTIERLIERFEKVHKVIVYSKESKEILLLNWYKYNWTKSEKFLTGLDKSIREVKDKEFKRYLLEIRSNADTVSIPYANGMDTTVTFSFTDIYSKDNNRDLNKDNKDISKDNSKNNSKGKNKIPKKSKVEIYYPNDEKLNQTFMDFLDMRNQMKKSMTDRAISLAMSKLQKLSQGDNEVAIQILEQSIFRNWIGLFPLNDDNSSSRDGGSENRQSQFEYLLNSIKEDEENDQNRS

SEQ ID NO: 60 - protein, 96 aa, Unidentified
  REGION 1..96: note = Bacteriophage strain LSP1
MAGIIINGKKYVLSNQDLLCEAVISRKGALFDFNTQTKRLYYAKGKTFILQTTYDLAGDDEYKVLSQKEAHAFMNKYPSGIHETVYKRYFGEPEEM

SEQ ID NO: 61 - protein, 108 aa, Unidentified
  REGION 1..108: note = Bacteriophage strain LSP1
MKVSKTIPEKKETGRDAAIAELFCEYVKSMEDKPRHTTAAETEADMYDQFCVGAFPDNVKLQSELYDKMMNVAVEFEESGFIAGFKTAMKLLDNVALLRGRETTVWQA

SEQ ID NO: 62 - protein, 57 aa, Unidentified
  REGION 1..57: note = Bacteriophage strain LSP1
LGIQKGTKLTKNPKNNTFKLRLDEITMDKLEFLSKKQQISKADVIRKGIDVQFEQEK

SEQ ID NO: 63 - protein, 284 aa, Unidentified
  REGION 1..284: note = Bacteriophage strain LSP1
LLILQQEFEKGTVRNVNANNLVHIGEADISVKEYNGQRVVTFKDIDVVHGRPEGTARKRFSDNRERFIEGKHFIKITASEFRTTIGNMDLRQQNDVNLVTERGYLMLVKSFTDDLAWEVQDQLVDNYFRVRQLVDDLSPQMKLLYGLCDQLAQAEREAKEAKKIADSAKEVASKAYEVTENIKDAVKPILDNWREEINTKFNRIQKSCGTEFNVLRNEMYSELECRAGCDLGTRLRNKKQRMSDSGCTKTEIKNTNRMDIIDEDKKLREIFSKIVSEYEIKYCA

SEQ ID NO: 64 - protein, 244 aa, Unidentified
  REGION 1..244: note = Bacteriophage strain LSP1
MYELVKVNYETEPSTVSARELHKKLEVKYDFARWVNSNFKEFVENVDFSRGHIDVIGNQYGGTQTIDDYNLSIDMAKHLCLMSKSEKGKMCRQYLIDLEKAWNTPEQIMARVLKMADKAIDKLKMDKAILLEDVKRMKPKEIFADAVSVSNTSILIGELAKILKQNGIDIGQNRLFEWMRGNGYLIKRKGTDYNMPTQKSMELDLFEIKERTIANPDGSVRITKTVLVSGKGQQNFINKFLVVA

SEQ ID NO: 65 - protein, 57 aa, Unidentified
  REGION 1..57: note = Bacteriophage strain LSP1
MAAVGFCCRKGGLRFTRTATSGNEVVMWSRVRDTWQIWACTQVRTMILCKRDGNIRI

SEQ ID NO: 66 - protein, 215 aa, Unidentified
  REGION 1..215: note = Bacteriophage strain LSP1
MSEEKMREPMIYAAICGVMEDIKAVGKDALNKTQGFKYRSIDAVMNALNPAMIKHKIFCAPEVIEQIREERTTGKGSTLIYSVCKMKYRFFAVDGSYVDAVVVGEGMDSGDKATNKAMSVAFKYACFQTFCIPTENLIDDPDADTPELNRKNSGNKQSKDLDNLRNELSNELVRTGYTWSSVIKKYKVDSVEHLTSLQLKDCLKKIKSVEDKAVS

SEQ ID NO: 67 - protein, 311 aa, Unidentified
  REGION 1..311: note = Bacteriophage strain LSP1
VEKAIQDKLAEYEGIVFTEDSKASARKEVASLRAEKKSLQDNMRDAKTQYMKPWENFEAQAKKLIGLYDEPIDLINGQIQAFEKKRIAEKKQLIQKIYKCVPEDLQVYISLERIYNPKWENATYKEKDIKADIAEFVHKTENDLNTITSMQSEAVEKALNIYKDSLSLTDAITYINNYEQQKQQIFAREQEKQRQEEVEKIRREEREKMLAEQRAREAQEAALRQAEAEKEAVLRQAEAEKEEALRQAEAEKIAAVEQAREDAAQEVIESFIPDIEGEENLYEYRISLSNDAKEKLEMYMDSVGIEWEMIE

SEQ ID NO: 68 - protein, 60 aa, Unidentified
  REGION 1..60: note = Bacteriophage strain LSP1
MICQIEDQMVVDEAWDWNESMLPERVRLKRERQAYEEAERENRNEWYSSSNYAGECEDYL

SEQ ID NO: 69 - protein, 59 aa, Unidentified
  REGION 1..59: note = Bacteriophage strain LSP1
MIITTIMCEVEDCAYRIDGSCSKSLICITPKTFSAFDRGQREWGPVCEDYKEVDNDLSD

SEQ ID NO: 70 - protein, 59 aa, Homo sapiens
MEQEVLKILSSGKCLDRYKRVLHLLNGEDSDKYKKKQPPRKVFLGWGCFFKNKSVLIIT

SEQ ID NO: 71 - protein, 79 aa, Homo sapiens
MFKACKAELVPTKQAALELGVSTATLRSWMKHQHINIGVYDKKDGSSRAACYIYRSMLDAEKRRLMGDKYGARSFKDSV

SEQ ID NO: 72 - protein, 53 aa, Unidentified
  REGION 1..53: note = Bacteriophage strain LSP1
VTQQQLKQAADPLNTKPLKSDNIELGSVIPLSDGRKGIVVEEVELCSKHVKQS

SEQ ID NO: 73 - protein, 178 aa, Unidentified
  REGION 1..178: note = Bacteriophage strain LSP1
MNNLRKTTITTLEIAEMMEVSHADILKKLEGRKDRKGYIQVISEGQMSVADYFVKSVYIDAQGKERPCYEVTKLGCDFLANKFTGEKGVLFTARYVKRFHDMENAIQQKVYETKATSLGEVASYTKEMDRRMEKQGTEPWKICEAFKMVSEQFGIILPVDFVKVPKYNQMNLFNMEEK

SEQ ID NO: 74 - protein, 56 aa, Unidentified
  REGION 1..56: note = Bacteriophage strain LSP1
MLTESKRNSIGEVIDKLTRLDKVGMMIIMSNAMALLARQELDKDNASQDTTKQTDL

SEQ ID NO: 75 - protein, 134 aa, Unidentified
  REGION 1..134: note = Bacteriophage strain LSP1
LTVGERLRYLRKTKLGYKNCDDFGVRIGLSGSNIRNIETGRINLTDRVVSDICNVFSVNENWLRNGGDDKDIFLELNEDEELAMCTQMLIDSEVDDTIANMIKKFICIYEKLDTDSKKVLMNIADEMINNLKKE

SEQ ID NO: 76 - protein, 38 aa, Unidentified
  REGION 1..38: note = Bacteriophage strain LSP1
MNDKSEVLKLIQKLDEEKEKKFIIQILIMIKTHLSGRH

SEQ ID NO: 77 - protein, 357 aa, Unidentified
  REGION 1..357: note = Bacteriophage strain LSP1
MKNPNGYGGIVDLGKNRRRPIAVRVPNGVKLTEDCREIPQYKYLGYFERTPQGKKDAQLLLAQYNAGMPVKTSNVNSCPTFAERYKLWLDRHLKHVESQKGTVSNQLSGSYNAAFKRCQTVHSKKINCIKYQDIQDIADNAASMSSSSVHNLNIVLFNVFDFARKQKYIEENFIGDIEFNYKKKANSIHETFTEDELSILWKNADDANTRIILIMIYTGLRIEELLSMHCKNVFLAEKYMIGGIKTEAGKDRIIPISNKILPFISQLYCENLYLIRHGKKRYSRPNFLNNIWNPTMNKFNLNHLPHDTRYTCATLMDRANVNQNCIKEILGHSKGDITSKVYIKKNLEDLICAINMI

SEQ ID NO: 78 - protein, 445 aa, Unidentified
  REGION 1..445: note = Bacteriophage strain LSP1
MNIFSSYKVKIKNYNKIFEETVRIYREAVSFFIDICDKEWDMLEPLKNLERCRKIEELSLHTKKNLNPKYDFNSKFYKMPGYLRRSAINTAAGCYSSYRSNLKNWEGNQLGRKPRLQLDRNVMPTLYKDNMYIRTGDDTARIKIFHRNDWIWLDIRLNNQDVRYIKSHCAFKKEYVPTLKKQGKCWYLVFPFEDKVKFEDVDIQDQTICSVDLGLNNNATCSIMQSDGTVAARKFINLATEKDHLYKALNRVKKVQQNGVRKTPILWKHVNDINTDISRKTAKQIIDFVVLHNADVIVFEHLDTSGKKRGSKKQRLHLWRKQEIQKIVEHRAHILGIRISRICAWNTSRLAFDGSGKVERGEYYQNGERKYNYSICLFKNGKQYNCDLNASYNIGARYFIRGLLKSDSVRKRLPSQTKDSDYGTGTTRTLSTLIRLNADLCRKTV

SEQ ID NO: 79 - protein, 16 aa, Unidentified
  REGION 1..16: note = Bacteriophage strain LSP1
TGTCTTTCTGCTGATA

SEQ ID NO: 80 - protein, 74 aa, Unidentified
  REGION 1..74: note = Bacteriophage strain LSP1
MDKIKKYVERFFKSKPEDRNYSLTTEDVFALFKEIQSADGNGLFYAIVSLFRYGYAKGYRVAQAEMKKKARKTA

SEQ ID NO: 81 - protein, 197 aa, Unidentified
  REGION 1..197: note = Bacteriophage strain LSP1
LRVWGSKKIKENLIMVVKKIQPVELSANLVFRLKNLYHAKLDEFIAIVDNKREILFVNKKVPQEDIDGFLEVILFPEYGVADNEAKGPFLDYVYYKYGHHTCRELLDAHTWRMKQKTEQKAKDTAKTIIPLIEKQIDSEIPIANYDEALLQQIYCAGYDRKKKTPKNITDFGSVYLFYLGCLVGAGLAKGESYYGQD

SEQ ID NO: 82 - protein, 96 aa, Unidentified
  REGION 1..96: note = Bacteriophage strain LSP1
MAGIIINGKKYVLSNQDLLCEAVISRKGALFDFNTQTKRLYYAKGKTFILQTTYDLAGGDEYRVLSQKEAHAFMNKYPSGIHETVYKRYFGEPEEM

SEQ ID NO: 83 - protein, 104 aa, Unidentified
  REGION 1..104: note = Bacteriophage strain LSP1
MKVSRTIPESRDAAIAELFCEYVKSMEDKPRHTTAAETEADMYDQFCVGAFPDNVKLQSELYDKMMNVAVEFEESGFIAGFKTAMKLLDNVALLRGREAAVWQA

SEQ ID NO: 84 - protein, 257 aa, Unidentified
  REGION 1..257: note = Bacteriophage strain LSP1
MHKIKDISSQPLVEWGGQVVITTSQLAEVYGTTTKNVSNNFSRNKDRFTEGKHYFVLQGEELKEFMRVSAESGLPTTTSIAYLWTRRGASRHCKILGTDKAWEQFDYLEDNYFDRQNRTPQFPTTIQGQIQLLAQGHVELEAKIDSVKADLEQFKNDMPILGVEESLITSAVKKRGVECLGGKQSNAYQDKSLRGRLYSDLHRELRRQFGGISTYKAIKRNQTNTAVMIIQNYVPPLVLAETIEIINAQQTLESLGK

SEQ ID NO: 85 - protein, 40 aa, Unidentified
  REGION 1..40: note = Bacteriophage strain LSP1
LKEASHSDMIDLSDGKLLISIIEVANFGWGATSIFYSVHS

SEQ ID NO: 86 - protein, 194 aa, Unidentified
  REGION 1..194: note = Bacteriophage strain LSP1
MSKIGQVLTSIEVSQMVGKDHSKLLRDIRNYVSQLGEAKIGLSDFFTESTYITEQNKTMPCYLVTKKGCEFIAHKLTGQKGTEFTARYINRFHEMEDELTIINPSDISLGELASYLKIMDRKFLKIVLDLCTPIIYNKLCEDKRREGNKCLHAQDAHLYKMYHVQKSLIFVSQRQKSRILKTAQNNYSFPEQTL

SEQ ID NO: 87 - protein, 54 aa, Unidentified
  REGION 1..54: note = Bacteriophage strain LSP1
  SITE 54: note = misc_feature - Xaa can be any naturally occurring amino acid
MSQMGRPKSDNPKSEQVTVRLDREHSEILKEYCEKEKVEKAEAIRRGIRKLKEX

SEQ ID NO: 88 - protein, 404 aa, Unidentified
  REGION 1..404: note = Bacteriophage strain LSP1
MSIFDFFHKKQTIPHHTCETIKRQIQILTESINLVNDSCDVQTVLKQYDITCQTLEHLSKYTDEEMQAAGFKTLGSLSESLSNILADKENIINQAIQRNFDKEIDSLTTTDGKAKALDTLYNSIRIMDGLDSVNISFLDALYQERKNNLCITIQFGQFTPLPSKQFKTQTGLDIESEEYVQLSTSGDENVCPMCAQFEGKIFPVNDAPKLPLCPSCACAYMHYYKNDLPSNVVISHKNDFILPSKHTPLFYKHQQIIYNEPDINKQIRLCENDLNKLSEFMTPYINANFNAPQELVCRDLLPELYMRLGKWDKAEKAIKRCIEANAYYPDNGSEELLYLATFRKVATQAISYITQNPGCLQRNIYKALPYETDEKECLKHFLRNSLLITKEKFNNTNKLYIPKA

SEQ ID NO: 89 - protein, 211 aa, Unidentified
  REGION 1..211: note = Bacteriophage strain LSP1
MYYIIPFFYITCYNIPKTEDMSMRKIKIIVIMCCIIFGTCVGCGVSQKDYDKLQTEKNELEEELEASRQQLETLKQDYEKTKKELLDEKTQKLDEDRKHSFARAWAATYFGEDCLVLIDGEKYLQLVSQEPYSATSEGVQAVYKQCLNSMSGLGVYVDQIKYNKIGLKFQQTNGEELIEFVLKRENGTYVLDSISGNLVQVPILLAVLEKY

SEQ ID NO: 90 - protein, 71 aa, Unidentified
  REGION 1..71: note = Bacteriophage strain LSP1
MKEEYDIESLNPRKNPYVKRLKKQITINIDNDTVDFFKVQSEESGIPYQTLINLYLSDCAKNKKKLQMSWK

SEQ ID NO: 91 - protein, 105 aa, Unidentified
  REGION 1..105: note = Bacteriophage strain LSP1
MEYISFEWDENKNKINKAKHGIDFEEAATVFYDDDAIMFDDPEHSMEEERFLILGITKHENLCIVSHCYRGDDNIIRIISARKATKSEIKTYNRYVGGEINERGV

SEQ ID NO: 92 - protein, 138 aa, Unidentified
  REGION 1..138: note = Bacteriophage strain LSP1
MYMAVRNIEETRAEAVKRLEVLKVHKNVLNEFKGEEKLNKSEHMGILYWLDDEEQAMVNEFEKEHEVVVYHVIHQLTNIGDLYSLLYVKRDNSEWELDREDLEEGQALAYVINKKMPDCSEFGRIGVKSSIGGVIRIW

SEQ ID NO: 93 - protein, 267 aa, Unidentified
  REGION 1..267: note = Bacteriophage strain LSP1
MRFTNSPLVVYRKISPNKTSPRNHKIDTITIHCMAGNLSIETCGNVFASSSRKASSNYGIGTDGRIALYVEEKDRSWASSNAKNDHRAVTIEVANDGGAPDWHVSNMAIASLIELVTDICKRNSIKKLVWSTNKTDRINHLHDCNMTVHRDFAAKACPGNYLYNLHSYIAEQVNERLGVKTAPTNVAITPESNPYKEPTTNIRKGTKGEGAKWVQWCLWRFGLLDKSEIDGVIGSKSDMAIREAQRRLGLTVDGIVGKNTRQTFKQV

SEQ ID NO: 94 - protein, 130 aa, Unidentified
  REGION 1..130: note = Bacteriophage strain LSP1
MDFFINNWYVMVAVTAILAGAGYSVYIFIKKSSTEQLKSLKEWMLWAVTKAEKELGSGTGKLKLRYVYDMFITKFPWLRNTISFEIVSMMVDDALEEMRIMLERNKAVKDFVSGFSEERKDRLGVDLKLL

SEQ ID NO: 95 - protein, 88 aa, Unidentified
  REGION 1..88: note = Bacteriophage strain LSP1
MDIILIFIAVFLLAFIVIMLVLYYITGAIPDTLCTCVFTVCGGECGIMGWIKTTKERQKERQYELEDRKYQEDMEKQSDNSFEENMSE

SEQ ID NO: 96 - protein, 109 aa, Unidentified
  REGION 1..109: note = Bacteriophage strain LSP1
MDNPVTHRELEQFKELMESENDRLKEENDRQNHRINNLEESIKEISNLTAATQKLADNMENMLKVQEQQSNRLAKIENRDGEKWRKAMAYIGTAILGAVLAIVFAKIGL

SEQ ID NO: 97 - protein, 143 aa, Unidentified
  REGION 1..143: note = Bacteriophage strain LSP1
MRINKRISTPNFSFGVDMRSFRAINSNLAGVYYLGTGTSFDIKSKFPDSYQNFTVDNFIAGGLSGNVVAAKSYTDGISGVPVARANGFSISKSYNAQTGVLEISGNTQQIRQGYDKAVNITYGNAAQTIQTSCWLVIGEIQQP

SEQ ID NO: 98 - protein, 597 aa, Unidentified
  REGION 1..597: note = Bacteriophage strain LSP1
MKYYTSDNPVFSDKILIVERADLVNYENKTKSEKQLLQNDLVLKKRMDKYLGSDDNTEGPEFAEGIQSEDVIGAINEVFQLGSEKKKQLAENLTALGITSSANETWEQLLGKVLDLIDPSKDTVTAETLLSGYTAHNAAGTKITGTLADKTGTADYNAEASIDGTNKRIKLKVPATGKYGVGNYLYAAYTTIANLIGLTAAKLVKGNTILGITGSNNNMDTSGADAGAGDILAGKKAGVKGSLIIGTMPNKGAWTGATTGNGNVVIPAGYHNGQGHVSGAGAYNAGVSAADGRVNADSANYRGGYNAGVSATKKGTAGAGDVLSGKTFTNGSSVGASGTMPNKGAWTGATTGSGNVTIPAGYHNGQGHVSGAGAYNKGVTDADARVNANSANYKGGYNAGVSAADGRVNADSANYKGGYNAGVSATKKGTAGAGDVLSGKTFTNSSNVETSGTMANKGSTTQDATATQDDTYTYLTVPVAGYYNTASKLRTKNSNLEHNEVYYLGSYLRSEKTVNVAQKLPNLYSNLTADNFIVMPGKIGWSQGAAEQHSGSGNWYTEYNNSTGILTFYNSYSASGHGIAYTSENKVYVIIGNIESV

SEQ ID NO: 99 - protein, 130 aa, Unidentified
  REGION 1..130: note = Bacteriophage strain LSP1
MSETNKNVVITKAARMKLVKARAGAITLPKVVGMAFGNGGVETDGTVIEPPDSQAALKNELYRKPIDGYTFPEDTICRYECTLIGNELAGEEISEIGLYDENGDIICIKNFTRKGKDDDVEQTYVLEDIF

SEQ ID NO: 100 - DNA, 16 bp, synthetic construct
  misc_feature 1..16: note = Primer
  source 1..16: mol_type = other DNA
gtttgatcctggctca

SEQ ID NO: 101 - DNA, 16 bp, synthetic construct
  misc_feature 1..16: note = Primer
  source 1..16: mol_type = other DNA
gtgccagcmgccgcgg

SEQ ID NO: 102 - DNA, 20 bp, synthetic construct
  misc_feature 1..20: note = Primer
  source 1..20: mol_type = other DNA
ccgtcaattcmtttragttt

SEQ ID NO: 103 - DNA, 16 bp, synthetic construct
  misc_feature 1..16: note = Primer
  source 1..16: mol_type = other DNA
taccttgttacgactt

SEQ ID NO: 104 - DNA, 26 bp, synthetic construct
  misc_feature 1..26: note = Primer
  misc_feature 21..26: note = Each "n" is independently a, g, t or c.
  source 1..26: mol_type = other DNA
gccggagctctgcagatatcnnnnnn

SEQ ID NO: 105 - DNA, 20 bp, synthetic construct
  misc_feature 1..20: note = Primer
  source 1..20: mol_type = other DNA
gccggagctctgcagatatc

SEQ ID NO: 106 - DNA, 17 bp, synthetic construct
  misc_feature 1..17: note = Primer
  source 1..17: mol_type = other DNA
gttttcccagtcacgac

SEQ ID NO: 107 - DNA, 17 bp, synthetic construct
  misc_feature 1..17: note = Primer
  source 1..17: mol_type = other DNA
caggaaacagctatgac

SEQ ID NO: 108 - DNA, 34 bp, synthetic construct
  misc_feature 1..34: note = Primer
  source 1..34: mol_type = other DNA
ggaattccatatgagatttaccaatagtccgctg

SEQ ID NO: 109 - DNA, 34 bp, synthetic construct
  misc_feature 1..34: note = Primer
  source 1..34: mol_type = other DNA
ccggaattccacttgtttaaatgtctgacgtgta

(19) United States
(12) Patent Application Publication
(10) Pub. No.: US 2024/0035001 A1
(43) Pub. Date: Feb. 1, 2024

(54) BACTERIOPHAGE INFECTING DIABETES-INDUCING BACTERIUM AND USE THEREOF

(71) Applicants: NATIONAL INSTITUTE OF ADVANCED INDUSTRIAL SCIENCE AND TECHNOLOGY, Tokyo (JP); AJINOMOTO CO., INC., Tokyo (JP)

(72) Inventors: Keishi KAMEYAMA, Kawasaki-shi (JP); Hideyuki TAMAKI, Tsukuba-shi (JP); Hiroyuki KUSADA, Tsukuba-shi (JP); Yoichi KAMAGATA, Tsukuba-shi (JP)

(73) Assignees: NATIONAL INSTITUTE OF ADVANCED INDUSTRIAL SCIENCE AND TECHNOLOGY, Tokyo (JP); AJINOMOTO CO., INC., Tokyo (JP)

(21) Appl. No.: 18/356,448

(22) Filed: Jul. 21, 2023

Related U.S. Application Data
(63) Continuation of application No. PCT/JP2022/002100, filed on Jan. 21, 2022.

(30) Foreign Application Priority Data
Jan. 22, 2021 (JP) 2021-009193

(51) Int. Cl. C12N 7/00; C12Q 1/04; C12N 9/36; A61K 35/76

(52) U.S. Cl. CPC C12N 7/00; C12Q 1/04; C12N 9/2462; C12Y 302/01017; A61K 35/76; C12N 2795/14071

(57) ABSTRACT

A bacteriophage which consists of the nucleotide sequence represented by SEQ ID NO: 1 or a nucleotide sequence having 90% or more identity thereto, as its genome, is capable of infecting and lysing a diabetes-inducible bacterium belonging to Fusimonas intestini,containing a cyclic single-stranded DNA.

### CROSS REFERENCES TO RELATED APPLICATIONS

This application is a continuation of International Patent Application No. PCT/JP2022/002100, filed on Jan. 21, 2022, and claims priority to Japanese Patent Application No. 2021-009193, filed on Jan. 22, 2021, both of which are incorporated herein by reference in their entireties.

### REFERENCE TO A SEQUENCE LISTING

In accordance with 37 CFR § 1.833-1835 and 37 CFR§ 1.77(b) (5), the specification makes reference to a Sequence Listing submitted electronically as a .xml file named “548961US_ST26.xml”. The .xml file was generated on Jul. 13, 2023 and is 216,947 bytes in size. The entire contents of the Sequence Listing are hereby incorporated by reference.

### BACKGROUND OF THE INVENTION

### Field of the Invention

The present invention relates to bacteriophages that infect a bacterium capable of inducing the onset of diabetes (hereinafter also to be referred to as “diabetes-inducible bacterium”) and constituent elements thereof, as well as use thereof. More particularly, it relates to novel bacteriophages that can infect a diabetes-inducible bacterium belonging to Fusimonas intestini and lyse the same, lytic enzymes derived from the phage, as well as suppression of its host bacterium using the same and prophylaxis and treatment of diabetes caused by the host bacterium.

### Discussion of the Background

Diabetes is a refractory disease that causes various complications since metabolic disorders including chronic hyperglycemia persist as a main pathology. Diabetes is classified based on the causal factor into type 1 diabetes, type 2 diabetes, diabetes due to genetic abnormality, diabetes associated with other disease or conditions, gestational diabetes and the like. Type 1 diabetes and type 2 diabetes are multi-factor disease developed by single nucleotide polymorphism (SNP), which is one of genetic polymorphisms rather than gene abnormality, added with various environmental factors. While what causes the onset of diabetes is not completely clear, it is generally considered that type 2 diabetes is developed when people genetically prone to diabetes (genetic factor) acquire a lifestyle easily leading to diabetes (environmental factor).

As one of the environmental factors involved in the onset of diabetes, involvement of enteric bacteria has been clarified in recent years. For example, it has been reported that administration of antibiotics, probiotics or prebiotics to an animal model of type 1 or type 2 diabetes improves conditions of diabetes.

Kameyama et al. comprehensively examined bacterial flora in the feces of obesity model mice having an abnormally high level of fasting blood glucose and compared with that of normal mice. As a result, they found that a specific bacterium is predominantly present therein, succeeded in isolation of this bacterium from cecal contents of an obesity model mouse, and designated as strain AJ110941 (see WO 2013/146319 and Kameyama, K. and Itoh, K., Microbes Environ., 29(4): 427-430(2014), which are incorporated herein by reference in their entireties). The present inventors carried out phylogenetic analysis and detailed analyses of morphological, physiological and biochemical characteristics for strain AJ110941, and identified that the strain is a bacterium of a novel genus and species belonging to Lachnospiraceae and designated as Fusimonas intestini (see Kusada, H. et al., Sci. Rep., 7: 18087 (2017), which is incorporated herein by reference in its entirety).

Kameyama et al. also colonized the bacterium in the intestine of a germ-free obesity model mouse and investigated phenotypes of the mouse. As a result, they found that the bacterium causes reduced insulin secretory ability and hyperglycemia in the mouse and identified that the bacterium is a diabetes-inducible bacterium (see WO 2013/146319 and Kameyama, K. and Itoh, K., Microbes Environ., 29(4): 427-430(2014), which are incorporated herein by reference in their entireties). Furthermore, Patent Literature 1 also describes a method of detecting the bacterium using a 16S ribosomal RNA sequence specific to the species, and a prophylactic/therapeutic vaccine using a processed material of the bacterial cells.

While antibiotics are mainly used for eradication of pathogenic enteric bacteria, no antibiotic capable of selectively eradicating Fusimonas intestini is known. Since an enteric bacterial flora plays an important role in homeostasis of its host including intestine immunity, we must be careful to use an antibiotic for the prophylaxis/treatment of lifestyle-related diseases, which are not directly life-threatening diseases, also taking a risk of emergence of resistant bacteria into account.

In the meantime, in recent years, phage therapy, which utilizes a bacteriophage (also to be simply referred to as “phage”) for the control of a pathogenic bacterium such as multidrug-resistant bacterium, has attracted attention (see, e.g., JP-A-2011-50373, WO 00/69269, and WO 2007/007055, which are incorporated herein by reference in their entireties). While a phage attacks (infects and kills) a bacterium, it is a virus harmless to human. A wide variety of phages occurs in nature, phages that adapt enteric bacteria also exist in their intestine. A phage recognizes and binds to a receptor on the surface of its host bacterium, injects the viral genome into the bacterium, produces a large amount of daughter phages utilizing the host's system, and disrupts the cell wall by lytic enzymes to kill the bacterium. Since a phage has a high host specificity, it has less impact on a resident enteric bacterial flora. Also, a risk of emergence of a resistant bacterium is lower as compared with an antibiotic. Furthermore, it is advantageous in that it easily proliferates and a large amount of phages can be prepared at a low cost. In the United States, a phage spray, which is sprayed onto the surface of meat for preventing listerial food poisoning, has already been approved as a food additive by the U.S. Food and Drug Administration (FDA). Phage preparations designed to clinical application have also been rapidly developed in Europe and the United States.

However, it is not easy to isolate and identify a phage strain capable of infecting and lysing a pathogenic bacterium of interest, due to its high host specificity, and a lytic phage against a diabetes-inducible bacterium belonging to Fusimonas intestini such as strain AJ110941 has never been reported.

### SUMMARY OF THE INVENTION

Accordingly, it is an object of the present invention to isolate and identify a novel bacteriophage capable of infecting and lysing a diabetes-inducible bacterium belonging to Fusimonas intestini, and provide a suppressor of a diabetes-inducible bacterium belonging to Fusimonas intestini or an agent for the prophylaxis and/or treatment of diabetes caused by the bacterium.

To carry out the above-mentioned object, the present inventors tried to isolate a bacteriophage infecting strain AJ110941 from the strain. At first, a phage fraction was purified from fractionated solutions of the culture supernatant of strain AJ110941 by density gradient centrifugation. Electron microscopy revealed that a bacteriophage in a typical form of the Caudovirales, which has a structure consisting of head and tail (head-tailed structure), is present therein. Then, a genomic nucleic acid was extracted from the purified phage solution and treated with various nucleases, which revealed that the phage genome is a single-stranded DNA. The genomic DNA was amplified by PCR and cloned into a vector, and its genomic sequence was determined. As a result, the phage genome was a cyclic single-stranded DNA whose genome size was about 62 knt. By ORF prediction and annotation, 98 ORFs were found out, some genes characteristic in phage such as tail fibers that play an important role in host specificity, and lytic enzymes were annotated. The genome size of the phage was extremely larger as compared with a general single-stranded DNA virus, whereas all of the Caudovirales having a similar viral shape comprise a linear double-stranded DNA, suggesting that the phage is an extremely new virus in systematics.

In fact, systematic analyses based on its genomic sequence demonstrated that the phage is phylogenetically completely different from known single-stranded DNA viruses. The present inventors designated this novel phage strain as strain LSP1.

Next, the present inventors added the bacteriophage strain LSP1 to a medium of Fusimonas intestini strain AJ110941 and cultured the same, in order to verify whether or not the phage causes lysis of the strain AJ110941. As a result, in the strain LSP1 addition group, the growth of strain AJ110941 was remarkably suppressed, and the disruption of the cellular morphology due to lysis of strain AJ110941 was confirmed by microscopy. The above-mentioned results demonstrated that stain LSP1 is capable of at least lysing strain AJ110941.

Furthermore, the host specificity of strain LSP1 was verified by infection test using various type strains belonging to the Lachnospiraceae other than Fusimonas intestini. As a result, strain LSP1 did not infect these type strains and was shown to be a phage capable of specifically infecting and lysing Fusimonas intestini.

The present inventors also cloned a DNA fragment of ORF92 having a high homology to endolysins from the genomic DNA of strain LSP1 into an expression vector, and a recombinant protein was produced using Escherichia coli as a host. When the protein was added to strain AJ110941 ab extra, it was revealed that it was rapidly lysed. Since an endolysin typically has a catalytic domain at the N-terminal side and a peptidoglycan substrate-binding domain at the C-terminal side, which determines host specificity, it was suggested that the recombinant endolysin derived from strain LSP1 can lyse a diabetes-inducible bacterium belonging to Fusimonas intestini, similar to LSP1 phage particles.

Furthermore, the present inventors demonstrated that the endolysin derived from strain LSP1 has a lytic activity against not only Fusimonas intestini but also various multidrug-resistant bacteria, and also clarified that it has a biofilm formation-inhibitory effect on drug-resistant bacteria that form a biofilm as one of their drug-resistance mechanisms.

That is, the present invention is as follows.

(1) A bacteriophage capable of infecting and lysing a diabetes-inducible bacterium belonging to Fusimonas intestini, comprising a cyclic single-stranded DNA, which consists of the nucleotide sequence represented by SEQ ID NO: 1 or a nucleotide sequence having 90% or more identity thereto, as its genome.

(2) The bacteriophage according to (1), comprising at least a nucleotide sequence encoding any of the amino acid sequences in the following (a) to (c):
(a) the amino acid sequence represented by SEQ ID NO: 99;
(b) an amino acid sequence having 95% or more identity with the amino acid sequence of (a);
(c) an amino acid sequence wherein 1 to several amino acids are substituted, deleted, inserted or added in the amino acid sequence of (a).

(3) The bacteriophage according to (2), further comprising 97 kinds of nucleotide sequences encoding any of the 97 kinds of amino acid sequences, respectively, in the following (d) to (f):
(d) 97 kinds of amino acid sequences represented by SEQ ID NOs: n, wherein n is an integer of 2-98;
(e) 97 kinds of amino acid sequences each having 95% or more identity with the respective amino acid sequences shown in (d);
(f) 97 kinds of amino acid sequences shown in (d), wherein 1 to several amino acids are substituted, deleted, inserted or added in one or more sequences thereof.

(4) The bacteriophage according to any of (1) to (3), wherein the diabetes-inducible bacterium is Fusimonas intestini strain AJ110941 (FERM BP-11443).

(5) The bacteriophage according to any of (1) to (4), comprising a cyclic single-stranded DNA, which consists of the nucleotide sequence represented by SEQ ID NO: 1, as its genome.

(6) A protein of any of the following (a) to (c):
(a) a protein consisting of the amino acid sequence represented by SEQ ID NO: 93;
(b) a protein comprising an amino acid sequence having 95% or more identity with the amino acid sequence of (a), and capable of lysing a diabetes-inducible bacterium belonging to Fusimonas intestini;
(c) a protein comprising an amino acid sequence wherein 1 to several amino acids are substituted, deleted, inserted or added in the amino acid sequence of (a), and capable of lysing a diabetes-inducible bacterium belonging to Fusimonas intestini.

(7) The protein according to (6), wherein the diabetes-inducible bacterium is Fusimonas intestini strain AJ110941 (FERM BP-11443).

(8) A nucleic acid encoding the protein according to (6) or (7).

(9) A method of producing the protein according to (6) or (7), comprising synthesizing the protein in an expression system comprising the nucleic acid according to (8) in a form capable of expressing the same.

(10) An agent for suppressing a diabetes-inducible bacterium belonging to Fusimonas intestini, comprising the bacteriophage according to any of (1) to (5), the protein according to (6) or (7), or a protein obtained by the method according to (9).

(11) The agent according to (10) for removing or reducing the diabetes-inducible bacterium from the intestine of an animal.

(12) The agent according to (11) for the prophylaxis or treatment of diabetes in the animal.

(13) A method of detecting a diabetes-inducible bacterium belonging to Fusimonas intestini in a sample, comprising contacting the bacteriophage according to any of (1) to (5) with the sample, and detecting infection of the bacteriophage with the diabetes-inducible bacterium.

(14) The method according to (13), wherein a lytic enzyme gene of the bacteriophage is inactivated.

(15) An agent for suppressing a bacterium resistant to one or more antibiotics, comprising the protein according to (6) or (7), or a protein obtained by the method according to (9).

(16) The agent according to (15), wherein the bacterium can form a biofilm.

(17) A method for the prophylaxis or treatment of diabetes in an animal, comprising administering to the animal the bacteriophage according to any of (1) to (5), the protein according to (6) or (7), or a protein obtained by the method according to (9).

(18) The bacteriophage according to any of (1) to (5), the protein according to (6) or (7), or a protein obtained by the method according to (9) for use in the prophylaxis or treatment of diabetes in an animal.

(19) Use of the bacteriophage according to any of (1) to (5), the protein according to (6) or (7), or a protein obtained by the method according to (9) in the manufacture of an agent for the prophylaxis or treatment of diabetes in an animal.

### Advantageous Effects of Invention

The bacteriophage of the present invention has an ability that infects and lyses a diabetes-inducible bacterium belonging to Fusimonas intestini, including strain AJ110941. Therefore, the prophylaxis and/or treatment of diabetes will become possible by suppressing intestinal colonization of the diabetes-inducible bacterium and the proliferation and physiological action thereof. The present invention enables a selective eradication of an enteric bacterium causing a diabetes.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 is an electron micrograph showing the morphology of LSP1 phage. The scale bar shows 100 nm.

FIG. 2 shows the genomic structure of LSP1 phage. The figure shows ORFs configuration, dinucleotide bias, GC content, and GC skew from outside.

FIG. 3 shows a phylogenetic tree of whole single-stranded viruses (A) and an enlarged view thereof around LSP1 phage (B).

FIG. 4 shows an effect of LSP1 phage on the growth of Fusimonas intestini strain AJ110941. -Δ-: medium alone was added; -♦-: inactivated strain LSP1 was added; -▪-: strain LSP1 was added.

FIG. 5 is microscope photographs showing lysis of Fusimonas intestini strain AJ110941 by LSP1 phage. They show that a medium alone was added; an inactivated strain LSP1 was added; and strain LSP1 was added from the left. The scale bar shows 10 μm.

FIG. 6 shows the enzyme activity of LysF derived from LSP1 phage against Fusimonas intestini. The time course change in the turbidity (OD₆₀₀) of Fusimonas intestini cultured broth after LysF addition is shown. -Δ-: buffer alone was added; -▪-: inactivated LysF was added; -●-: LysF was added.

FIG. 7 shows that LysF derived from LSP1 phage has a lytic activity against Fusimonas intestini. The cellular morphology of Fusimonas intestini after LysF addition was observed by microscopy. Nucleus was stained using SYBR Green I (green), cellular membrane was stained using FM4-64 (red). The scale bar shows 5 μm.

FIG. 8 shows lytic activities of a recombinant LysF against multidrug-resistant bacteria (Acidovorax sp. MR-S7, M2, M6). A. The time course changes of bacterial cell contents (OD₆₀₀ value) after LysF+EDTA or EDTA alone (control) was added to each bacterial cell suspension are shown. B. The cellular morphologies at 6 hr after LysF+EDTA or EDTA alone (control) was added were observed by microscopy. Nucleus was stained using SYBR Green I (green), cellular membrane was stained using FM4-64 (red). The scale bar shows 5 μm.

FIG. 9 shows inhibitory effects of a recombinant LysF on biofilm formation in multidrug-resistant bacteria (Acidovorax sp. MR-S7, M2, M6).

### DETAILED DESCRIPTION OF THE PREFERRED EMBODIMENTS

I. The phage of the present invention

The present invention provides a bacteriophage capable of infecting and lysing a diabetes-inducible bacterium belonging to Fusimonas intestini (hereinafter also to be referred to as “the phage of the present invention”). In the present specification, a “lytic phage” means any phage capable of infecting a host bacterium (namely, binding the bacterial surface and delivering its own genomic DNA into the bacterial cell) and lysing the host bacterium, when its purified phage particles are contacted with the host bacterium. Accordingly, the lytic phages may also encompass phages that cause lysogenization under specific conditions and can be integrated into the host genome as a prophage. Since a representative example of the lytic phages of the present invention, strain LSP1, was isolated from the culture supernatant of a pure culture of its host strain AJ110941, the possibility that it lysogenizes the host strain cannot be denied, but an open reading frame (ORF) having a homology to a repressor gene that is considered to be necessary for lysogenization has not been found in comparison with known viral genomes. In infection test of the purified phage particles, they lyse strain AJ110941. Thus, there is no conclusive evidence that the phage can cause lysogenization.

### (A) Diabetes-Inducible Bacterium

A diabetes-inducible bacterium belonging to Fusimonas intestini, which is a host of the phage of the present invention, is an anaerobic Gram-positive bacterium belonging to Firmicutes, Clostridia, Clostridiales, Lachnospiraceae, Fusimonas, a long bacillus and an enteric bacterium having a pili or flagellum-like structure. More detailed mycological characteristics of Fusimonas intestini are described in the above-mentioned WO 2013/146319 and Kameyama, K. and Itoh, K., Microbes Environ., 29(4): 427-430(2014), which are incorporated herein by reference in their entireties. Alternatively, a bacterium belonging to Fusimonas intestini can be identified by its 16S ribosomal DNA having 98% or more identity with the 16S ribosomal DNA of strain AJ110941. The strains with 16S ribosomal DNA having 98% or more identity with the 16S ribosomal DNA of strain AJ110941 include, for example, WT ctrl D2 F09, NOD dss A5 D06 and NOD dss A2 C04 clones described in PLoS One, 7: e30273 (2012), 16saw23-2 g06.p1k and 16saw23-1b01.p1k clones described in PLoS Biol., 5: 2177-2189 (2007), and the like.

In the present specification, the “diabetes-inducible bacterium” means a bacterium having an activity inducing either or both of insulin resistance and reduced insulin secretory capacity. The presence or absence of insulin resistance-inducing activity and reduced insulin secretory capacity-inducing activity can be determined by, for example, the methods described in WO 2013/146319, which is incorporated herein by reference in its entirety.

As a representative example of diabetes-inducible bacteria belonging to Fusimonas intestini, strain AJ110941 can be mentioned. The strain AJ110941 was internationally deposited under accession No. FERM BP-11443 in the National Institute of Advanced Industrial Science and Technology, the International Patent Organism Depositary (Central 6, 1-1-1 Higashi, Tsukuba, lbaraki) [currently, the National Institute of Technology and Evolution, NITE Patent Microorganisms Depositary (2-5-8, Kazusakamatari, Kisarazu-shi, Chiba)] under the Budapest Treaty (acceptance date: Nov. 30, 2011).

### (B) Bacteriophage

The phage of the present invention contains a cyclic single-stranded DNA, which consists of the nucleic acid sequence represented by SEQ ID NO: 1 that is a genomic DNA sequence of a representative strain thereof, strain LSP1, or a nucleic acid sequence having 90% or more, preferably 95% or more (e.g., 95, 96, 97, 98, 99% or more) identity thereto, as its genome.

As used herein, the “identity” means the proportion (%) of identical nucleotides to all overlapping nucleotide residues in the optimal alignment where two nucleotide sequences are aligned using a mathematical algorithm known in the pertinent technical field (preferably, the algorithm is such that a gap can be introduced into one or both of the sequences for the optimal alignment). In the present specification, the “identity of nucleotide sequence” can be calculated using homology calculation algorithm blastn of NCBI BLAST (National Center for Biotechnology Information Basic Local Alignment Search Tool), and under default Scoring Parameters (Match/Mismatch Scores=1,-2; Gap Costs=Linear).

The strain LSP1 has a genome size of about 62 knt and has 98 ORFs. When the ORFs located in order from the 5′-terminal of the nucleotide sequence represented by SEQ ID NO: 1 are called ORF1 to ORF98, ORF92 and ORF93 are assumed to encode the cell wall peptidoglycan-degrading enzyme endolysin and the transmembrane protein holin, respectively. Therefore, the phage of the present invention is considered to have a lytic mode of lysing host bacteria by the action of holin and endolysin lytic enzyme. In addition, ORF98 is assumed to encode a protein constituting the tail fiber that is important for host specificity and that interacts with and binds to receptors on the surface of host cells. Information relating to ORFs in the LSP1 genome is shown in Tables 1-1 and 1-2. This information is a summary of what is considered rational based on a comprehensive judgment of the results of analyses from many directions, using analysis software such as PHAST, PHASTER, BLAST, InterPro, and pfam.

TABLE 1-1

| ORF # | Direction | Start | Stop | Length (nt) | Pfam | InterPro | Gene description |
|---|---|---|---|---|---|---|---|
| ORF1 | −1 | 1 | 1353 | 1,356 | DUF2313 | IPR018755 | hypothetical protein |
| ORF2 | −1 | 1366 | 2484 | 1,119 | Baseplate J | IPR006949 | baseplate J-like protein |
| ORF3 | −1 | 2499 | 2935 | 438 | DUF2634 | IPR020288 | hypothetical protein |
| ORF4 | −1 | 2953 | 3336 | 384 | — | — | hypothetical protein |
| ORF5 | −1 | 3416 | 4663 | 1248 | Prophage tail | IPR01572 | hypothetical protein |
| ORF6 | −1 | 4623 | 5353 | 681 | LysM | IPR018392 | peptidoglycan-binding protein |
| ORF7 | −1 | 5357 | 10042 | 4,686 | Glycine zipper | IPR010090 | tail tape measure protein |
| ORF8 | 1 | 5559 | 5574 | 16 | — | — | attL |
| ORF9 | −1 | 10017 | 10166 | 150 | — | — | hypothetical protein |
| ORF10 | −1 | 10190 | 10783 | 594 | Phage TAC 5 | IPRO14986 | tail assembly chaperone protein |
| ORF11 | −1 | 10856 | 11320 | 465 | Phage tail tube protein | IPR018989 | hypothetical protein |
| ORF12 | −1 | 11334 | 12743 | 1410 | Phage sheath | IPR007667 | tail sheath protein |
| ORF13 | −1 | 12744 | 13013 | 270 | — | — | hypothetical protein |
| ORF14 | −1 | 13024 | 13899 | 876 | — | — | hypothetical protein |
| ORF15 | −1 | 13874 | 14209 | 336 | DUF5026 | IP8032207 | hypothetical protein |
| ORF16 | −1 | 14221 | 14685 | 465 | — | — | hypothetical protein |
| ORF17 | −1 | 14682 | 15038 | 357 | — | — | hypothetical protein |
| ORF18 | −1 | 15035 | 15445 | 411 | — | — | hypothetical protein |
| ORF19 | −1 | 15445 | 16083 | 639 | — | — | hypothetical protein |
| ORF20 | −1 | 16094 | 17122 | 1029 | — | — | major capsid protein |
| ORF21 | −1 | 17141 | 17563 | 423 | DUF2190 | IPR011231 | hypothetical protein |
| ORF22 | −1 | 17581 | 18903 | 1323 | — | — | hypothetical protein |
| ORF23 | −1 | 18919 | 19416 | 498 | serine protease [symbol] | IPR027924 | hypothetical protein |
| ORF24 | −1 | 19422 | 19604 | 183 | — | — | hypothetical protein |
| ORF25 | −1 | 19705 | 19893 | 189 | — | — | hypothetical protein |
| ORF26 | −1 | 19875 | 21458 | 1584 | [symbol] like F like protein | IPR006528 | [symbol] morphogenesis protein |
| ORF27 | −1 | 21531 | 22919 | 1389 | — | — | portal protein |
| ORF28 | −1 | 22931 | 24775 | 1845 | Terminase6 | IPR004921 | terminase |
| ORF29 | −1 | 24855 | 25106 | 252 | — | — | hypothetical protein |
| ORF30 | −1 | 25118 | 25357 | 240 | — | — | hypothetical protein |
| ORF31 | −1 | 25660 | 25773 | 114 | — | — | hypothetical protein |
| ORF32 | −1 | 25879 | 26538 | 660 | — | — | recombination factor protein |
| ORF33 | −1 | 26623 | 27345 | 723 | — | — | hypothetical protein |
| ORF34 | −1 | 27329 | 28039 | 711 | WbqC | IPR014985 | hypothetical protein |
| ORF35 | −1 | 28043 | 28702 | 660 | PIG-L | IPR003737 | N-acetylglucosaminylphosphatidylinositol |
| | | | | | | | deacetylase |
| ORF36 | −1 | 28708 | 29241 | 534 | — | — | hypothetical protein |
| ORF37 | 1 | 29216 | 29611 | 396 | — | — | hypothetical protein |
| ORF38 | −1 | 29630 | 30205 | 576 | — | — | hypothetical protein |
| ORF39 | −1 | 30324 | 30710 | 386 | — | — | hypothetical protein |
| ORF40 | −1 | 30732 | 30911 | 180 | — | — | hypothetical protein |
| ORF41 | −1 | 30955 | 31158 | 204 | — | — | hypothetical protein |
| ORF42 | −1 | 31254 | 31610 | 357 | — | — | tail protein |
| ORF43 | −1 | 31651 | 32349 | 699 | — | — | hypothetical protein |
| ORF44 | −1 | 32383 | 32550 | 168 | — | — | hypothetical protein |
| ORF45 | −1 | 32601 | 33533 | 933 | — | — | radical SAM protein |
| ORF46 | −1 | 33530 | 34009 | 480 | [symbol] | IPR007374 | hypothetical protein |
| ORF47 | −1 | 34082 | 34672 | 591 | — | — | hypothetical protein |
| ORF48 | −1 | 34677 | 34883 | 207 | — | — | hypothetical protein |
| ORF49 | −1 | 34910 | 35239 | 321 | — | — | hypothetical protein |
| ORF50 | −1 | 35309 | 35857 | 549 | DUF551 | IPR007539 | hypothetical protein |

[symbol]

indicates data missing or illegible when filed

TABLE 1-2

| ORF # | Direction | Start | Stop | Length (nt) | Pfam | InterPro | Gene description |
|---|---|---|---|---|---|---|---|
| ORF51 | −1 | 35870 | 36307 | 438 | — | — | hypothetical protein |
| ORF52 | −1 | 36340 | 36537 | 198 | — | — | hypothetical protein |
| ORF53 | −1 | 36534 | 36680 | 147 | — | — | hypothetical protein |
| ORF54 | −1 | 36677 | 37453 | 777 | PAPS reduct | IPR002500 | phosphoadenosine phosphosulfate reductase |
| ORF55 | −1 | 37606 | 37731 | 126 | | | hypothetical protein |
| ORF56 | −1 | 37700 | 38013 | 312 | — | — | hypothetical protein |
| ORF57 | −1 | 38157 | 38804 | 648 | Inhibitor G39P | IPR024424 | Loader and inhibitor of phage [symbol] |
| ORF58 | −1 | 38785 | 39666 | 882 | — | — | replication protein |
| ORF59 | −1 | 39825 | 40115 | 291 | — | — | hypothetical protein |
| ORF60 | −1 | 40103 | 40429 | 327 | — | — | hypothetical protein |
| ORF61 | −1 | 40549 | 40722 | 124 | — | — | hypothetical protein |
| ORF62 | −1 | 40859 | 41713 | 855 | ORF6N | IPR018873 | anti-repressor protein |
| ORF63 | −1 | 41881 | 42615 | 735 | AntA/ANT | IPR013357/IPR005039 | anti-repressor protein |
| ORF64 | −1 | 42637 | 42810 | 174 | — | — | hypothetical protein |
| ORF65 | −1 | 42863 | 43510 | 648 | EEF | IPR007499 | ssDNA-binding protein |
| ORF66 | −1 | 43507 | 44442 | 936 | DUF1351 | IPR009785 | hypothetical protein |
| ORF67 | −1 | 44453 | 44635 | 183 | — | — | hypothetical protein |
| ORF68 | −1 | 44619 | 44798 | 180 | — | — | hypothetical protein |
| ORF69 | −1 | 44815 | 44994 | 180 | — | — | hypothetical protein |
| ORF70 | −1 | 44963 | 45202 | 240 | — | — | hypothetical protein |
| ORF71 | −1 | 45175 | 45336 | 162 | — | — | hypothetical protein |
| ORF72 | −1 | 45377 | 45913 | 337 | Phage pRha | IPR014054 | hypothetical protein |
| ORF73 | −1 | 45966 | 46136 | 371 | — | — | hypothetical protein |
| ORF74 | 1 | 46301 | 46705 | 405 | [symbol] | IPR001387 | transcriptional regulator |
| ORF75 | −1 | 46713 | 46829 | 117 | — | — | hypothetical protein |
| ORF76 | 1 | 47192 | 48265 | 1074 | Phage integase | IPR052104 | integrase |
| ORF77 | −1 | 48563 | 49900 | 1338 | — | — | transposase |
| ORF78 | 1 | 49058 | 49073 | 16 | — | — | attR |
| ORF79 | −1 | 50144 | 50368 | 225 | — | — | hypothetical protein |
| ORF80 | −1 | 50355 | 50906 | 552 | — | — | hypothetical protein |
| ORF81 | −1 | 51043 | 51333 | 291 | — | — | hypothetical protein |
| ORF82 | −1 | 51321 | 51635 | 315 | — | — | hypothetical protein |
| ORF83 | −1 | 51658 | 52431 | 774 | ORF6N/ORF6C | IPR018873/IPR018878 | anti-repressor protein |
| ORF84 | 1 | 52459 | 52581 | 123 | — | — | hypothetical protein |
| ORF85 | −1 | 52600 | 53184 | 585 | Phage pRha | IPR014054 | anti-repressor protein |
| ORF86 | −1 | 53307 | 53468 | 162 | — | — | transcriptional regulator |
| ORF87 | 1 | 53715 | 54929 | 1215 | — | — | hypothetical protein |
| ORF88 | −1 | 55043 | 56678 | 636 | — | — | hypothetical protein |
| ORF89 | −1 | 55796 | 56011 | 216 | BrnA antitoxin | IPR025328 | antitoxin |
| ORF90 | −1 | 55995 | 56312 | 318 | BrnT toxin | IPR007460 | toxin |
| ORF91 | −1 | 56490 | 56906 | 417 | — | — | hypothetical protein |
| ORF92 | −1 | 57423 | 58226 | 804 | Amidase Z/PG binding1 | IPR002502/IPR092477 | N-acetylcinnamoyl-L-alanine amidase |
| ORF93 | −1 | 58310 | 58702 | 393 | — | — | phage holin protein |
| ORF94 | −1 | 58712 | 58978 | 267 | — | — | hypothetical protein |
| ORF95 | −1 | 59012 | 59341 | 330 | — | — | hypothetical protein |
| ORF96 | 1 | 59366 | 59797 | 432 | — | — | hypothetical protein |
| ORF97 | −1 | 59834 | 61627 | 1794 | — | — | tail fibre repeat-containing protein |
| ORF98 | −1 | 61643 | 62035 | 393 | DUF3731 | IPR022225 | tail-collar fiber protein |

[symbol]

indicates data missing or illegible when filed

(In the Tables, in “Direction”, ORFs encoded in chain consisting of the nucleotide sequence represented by SEQ ID NO: 1 are shown as “1”, and those encoded in - chain are shown as “-1”.)

In a preferred embodiment, the phage of the present invention is further characterized in that it contains a nucleotide sequence (i.e., ORF) encoding any of amino acid sequences in the following (a) to (c):
(a) the amino acid sequence represented by SEQ ID NO: 99;
(b) an amino acid sequence having 95% or more (e.g., 95, 96, 97, 98, 99% or more) identity with the amino acid sequence of (a);
(c) an amino acid sequence wherein 1 to several (e.g., 2, 3, 4, 5, 6) amino acids are substituted, deleted, inserted or added in the amino acid sequence of (a).

As used herein, the “identity” means the proportion (%) of identical amino acids to all overlapping amino acid residues in the optimal alignment where two amino acid sequences are aligned using a mathematical algorithm known in the pertinent technical field (preferably, the algorithm is such that a gap can be introduced into one or both of the sequences for the optimal alignment). In the present specification, the “identity of amino acid sequence” can be calculated using homology calculation algorithm NCBI BLAST (National Center for Biotechnology Information Basic Local Alignment Search Tool), and under the following conditions (expectancy=10; accept gap; matrix=BLOSUM62; filtering=OFF).

The amino acid sequence of the above-mentioned (a) corresponds to the amino acid sequence of a protein that constitutes the tail fiber encoded by ORF98 of the strain LSP1 and is assumed to be important for the host specificity of the phage strain.

When the phage of the present invention contains an ORF encoding the above-mentioned amino acid sequence (b) or (c), the protein composed of the amino acid sequence does not impair the host specificity for diabetes-inducible bacterium belonging to Fusimonas intestini.

The substitution of amino acids in the above-mentioned (c) is preferably substitution with similar amino acids. The “similar amino acid” means amino acids having similar physicochemical properties and, for example, amino acids classified in the same group such as aromatic amino acids (Phe, Trp, Tyr), aliphatic amino acids (Ala, Leu, Ile, Val), polar amino acids (Gln, Asn), basic amino acids (Lys, Arg, His), acidic amino acids (Glu, Asp), amino acids having a hydroxyl group (Ser, Thr), amino acids having a small side chain (Gly, Ala, Ser, Thr, Met) and the like can be mentioned. It is predicted that the substitution with such similar amino acids does not change the phenotype of the protein (that is, conservative amino acid substitution). Specific examples of the conservative amino acid substitution are well known in the technical field and are described in various documents (e.g., Bowie et al., Science, 247: 1306-1310 (1990), which is incorporated herein by reference in its entirety).

In another preferred embodiment, the phage of the present invention is further characterized in that it contains 97 kinds of ORFs the same or substantially the same as ORFs 1 to 97 of the strain LSP1. That is, the phage contains 97 kinds of nucleotide sequences (i.e., ORFs) encoding the amino acid sequences shown in the following (d) to (f):
(d) respective amino acid sequences represented by SEQ ID NOs: n, wherein n is an integer of 2-98;
(e) amino acid sequences each having 95% or more (e.g., 95, 96, 97, 98, 99% or more) identity with the respective amino acid sequences shown in (d);
(f) 97 kinds of amino acid sequences shown in (d), wherein 1 to several (e.g., 2, 3, 4, 5, 6) amino acids are substituted, deleted, inserted or added in one or more sequences thereof.

The amino acid sequences of the above-mentioned (d) correspond to the amino acid sequences of the predicted proteins respectively encoded by the ORFs 1 to 97 of the strain LSP1.

When the phage of the present invention contains each ORF encoding the above-mentioned amino acid sequence (e) or (f) and a protein is actually produced from the ORF, it has the same function as that of the corresponding protein of the strain LSP1. The substitution of amino acid in the above-mentioned (f) is preferably a substitution with a similar amino acid.

In a particularly preferred embodiment, the phage of the present invention is strain LSP1 containing a cyclic single-stranded DNA, which consists of the nucleotide sequence represented by SEQ ID NO: 1, as its genome.

The phage of the present invention can be obtained by recovering a phage fraction from sewage of general household and the like or drain from a sewage disposing faculty and the like, or a biological sample (e.g., feces, intestinal content etc.) derived from an animal wherein Fusimonas intestini is colonized in its intestine, using conventional methods, recovering a single plaque by a plaque assay method, and isolating a bacteriophage by filtration, density gradient centrifugation and the like. The phage can be further purified by methods known per se such as ultracentrifugation. Since Fusimonas intestini strain AJ110941 is internationally deposited as mentioned above, one of the phage of the present invention, strain LSP1, can be easily isolated from a culture solution of the deposited bacterium.

Whether the obtained bacteriophage corresponds to the phage of the present invention is determined by, after confirming that it has a head-tail structure in morphological observation using an electron microscope and has a genomic structure of cyclic single-stranded DNA by isolating genomic DNA, for example, obtaining a fragment containing ORF98 or the like that encodes a protein that constitutes the tail fiber by using, for example, a PCR method or the like, examining the homology with the corresponding ORF of the strain LSP1, determining the whole genome of those that satisfy the criteria of the phage of this patent (that is, sequence identity of 95% or more when aligned with blastx), and examining whether they have 90% or more identity with the genomic sequence of the strain LSP1.

The phage of the present invention can be proliferated using a general proliferation method of bacteriophage. For example, a large amount of the phage of the present invention can be prepared by culturing a host Fusimonas intestini (e.g., strain AJ110941 etc.), inoculating the phage of the present invention thereto after sufficiently proliferating same and continuing cultivation.

A host Fusimonas intestini can be cultured by, for example, as described in Patent Literature 1, performing standing culture using an anaerobic medium (e.g., EG medium, modified GAM broth etc.) under anaerobic conditions (e.g., at an oxygen concentration of 1 ppm or less) at 30-40° C., preferably about 37° C. The culture may be a liquid culture or solid culture. The timing of inoculation of the phage of the present invention is not particularly limited, for example, it is preferable to inoculate when the bacterium is grown up to a bacterial concentration of about 0.1 at OD₆₆₀. The culture can be performed until almost all of the bacterial cells are lysed (about 6-30 hr after inoculation).

The phage can be fractionated and purified from the obtained plaque or culture supernatant in the same manner as mentioned above. The purified phage obtained can be stored, for example, at 4° C. for about 1 month. It is also possible that the phage can be stably stored for a long time using methods known per se such as low temperature storage in a glycerin solution or salt solution; cryopreservation in liquid nitrogen, deep freezer, or dry ice; freeze-dry and the like.

### (C) Agent for Suppressing Diabetes-Inducible Bacterium

Since the phage of the present invention can specifically infect and lyse a diabetes-inducible bacterium belonging to Fusimonas intestini, the phage can be selectively eradicated from a subject in which the bacterium is present, by contacting the phage with the subject. Therefore, the present invention also provides an agent for suppressing diabetes-inducible bacterium belonging to Fusimonas intestini, comprising the phage of the present invention (hereinafter also to be referred to as the “suppressor of the present invention”).

Herein, “suppression” means suppressing at least proliferation of a host bacterium, preferably, reducing the number of the bacteria in a subject, more preferably, removing the bacterium from the subject.

The phage of the present invention can be formulated alone, or together with an additive acceptable pharmaceutically or for food or feed processing. Alternatively, the phage can be contained as a pharmaceutical additive or food or feed additive in a pharmaceutical composition or food or feed.

When the phage of the present invention is provided as a medicament or pharmaceutical additive, the medicament or a pharmaceutical composition containing the pharmaceutical additive can be formulated, for example, into powder, granule, pill, soft capsule, hard capsules, tablet, chewable tablet, quick-integrating tablet, syrup, liquid, suspension, suppository, injection and the like.

For example, compositions for oral administration include a solid or liquid dosage form, specifically tablet (including sugar-coated tablet, film-coated tablet), pill, granule, powder, capsule (including soft capsule), syrup, emulsion, suspension and the like. Such composition is produced by a known method, and may contain an additive generally used in pharmaceutical field, for example, excipient, binder, disintegrant, lubricant and the like. Examples of the excipients include animal and plant oils such as soybean oil, safflower oil, olive oil, germ oil, sunflower oil, beef tallow, and sardine oil, and polyhydric alcohols such as polyethylene glycol, propylene glycol, glycerol, and sorbitol, surfactants such as sorbitan fatty acid ester, sucrose fatty acid ester, glycerol fatty acid ester, polyglycerol fatty acid ester, purified water, lactose, starch, crystalline cellulose, D-mannitol, lecithin, gum arabic, sorbitol solution, carbohydrate solution and the like. Examples of the binders include hydroxypropylmethylcellulose, hydroxypropylcellulose, gelatin, pregelatinized starch, polyvinylpyrrolidone, poly(vinyl alcohol) and the like. Examples of the disintegrants include carmellose calcium, carmellose sodium, croscarmellose sodium, crospovidone, low-substituted hydroxypropylcellulose, cornstarch and the like. Examples of the lubricants include talc, hydrogenated vegetable oil, waxes, those derived from natural products such as light silicic anhydride, and derivatives thereof, stearic acid, magnesium stearate, calcium stearate, aluminum stearate, and the like.

Furthermore, sweeteners, coloring agents, pH adjusters, flavors, various amino acids, and the like can also be added to the above-mentioned composition. In addition, tablets and granules may be coated by a well-known method. A liquid preparation may be in the form of being dissolved or suspended in water or other suitable medium at the time of administration.

As the composition for parenteral administration, injection, suppository, and the like are used. Injection can be prepared, for example, by suspending or emulsifying the phage of the present invention in a sterile aqueous solution or an oily solution generally used for injection. As the aqueous solution for injection, for example, saline, isotonic solution containing glucose and other auxiliary agents, and the like are used. As the oily solution, sesame oil, soybean oil and the like are used. Suppositories used for rectal administration can be prepared by mixing the phage of the present invention with an ordinary suppository base.

Furthermore, when provided as a pharmaceutical product or pharmaceutical product additive, the phage of the present invention may be used in combination with other drugs such as antibiotics, antidiabetic drugs, and the like, depending on the target disease. The phage of the present invention and the concomitant drug may be formulated as a single composition (combination agent) or provided as separate compositions. When provided as separate compositions, the phage of the present invention and the concomitant drug can be administered to the subject at the same time or with time difference, by the same route or different routes.

When the phage of the present invention is provided as a food (or feed) or food additive (or feed additive), the food (or feed) or a food (or feed) containing the additive is not particularly limited as long as it is in an orally ingestible form such as solution, suspension, powder, solid molded product, or the like. Specific examples include supplements (powder, granule, soft capsule, hard capsules, tablet, chewable tablet, quick-integrating tablet, syrup, liquid, etc.), drinks (carbonic acid drinks, lactic drinks, sports drinks, fruit juice drinks, vegetable drinks, soy milk beverages, coffee drinks, tea drinks, powder drinks, concentrated drinks, nutritional beverages, alcoholic drinks, etc.), dairy products (yogurt, butter, cheese, ice cream, etc.), confectionery (gummi, jelly, gum, chocolate, cookie, candy, caramel, Japanese confectionery, snack, etc.), convenience foods (instant noodle, retort food, can, microwave food, instant soup or miso soup, freeze-dried food, etc.), oil, oil and fat foods (mayonnaise, dressing, cream, margarine, etc.), wheat flour products (bread, pasta, noodle, cake mix, breadcrumb, etc.), seasonings (sauce, processed tomato seasoning, flavored seasoning, cooking mix, seasoning soy sauce, etc.), and processed meat products (meat ham, sausage, etc.).

Where necessary, various nutritions, various vitamins (vitamin A, vitamin B1, vitamin B2, vitamin B6, vitamin C, vitamin D, vitamin E, vitamin K, etc.), various minerals (magnesium, zinc, iron, sodium, potassium, selenium, etc.), dietary fiber, dispersing agent, stabilizers such as emulsifier, sweetener, taste components (citric acid, malic acid, etc.), flavor, royal jelly, propolis, Agaricus and the like can be added to the above-mentioned food (or feed).

The amount of the phage of the present invention to be contained in the suppressor of the present invention may be, for example, 10⁴ to 10¹² phage particles (VLP), preferably 10⁵ to 10¹¹ VLP, per mL.

Bacterial cells or processed bacterial cells of other useful microorganisms can be further added to the phage of the present invention. Examples of such other microorganisms include, but are not limited to, lactobacillus belonging to Lactobacillus, Streptococcus, Leuconostoc, Pediococcus, Lactococcus, Enterococcus, Bifidobacterium, and the like, yeast, Bacillus, Clostridium butyricum, Aspergillus oryzae, and the like. These concomitant microorganisms can be contained in the suppressor of the present invention not only in the form of viable cells, but also in the form of dead cells or crushed cells, cell extracts, cell components, and the like, as long as the effectiveness thereof is found.

The amount of the concomitant microorganism may be, for example, 10⁴ to 10¹² colony forming units (cfu), preferably 10⁵ to 10¹¹ cfu, per mL.

The suppressor of the present invention can be used for, for example, removing or reducing a diabetes-inducible bacterium belonging to Fusimonas intestini from an animal in which the bacterium is colonized (or having a risk which the bacterium will be colonized) in its intestine. Such agent for removing or reducing the diabetes-inducible bacterium can be applied to human or other mammals, in a form of a pharmaceutical (including veterinary) composition, food or feed and the like.

Since a diabetes-inducible bacterium belonging to Fusimonas intestini has an ability inducing insulin resistance and/or reduced insulin secretory capacity, removal or reduction of the bacterium from the intestine in a mammal that has conditions of insulin resistance and/or reduced insulin secretory capacity caused by the bacterium improves these conditions, and exerts therapeutic and progression-suppressing effects on a patient (sick animal) that has already developed diabetes, or preventing and delaying effects on the onset of diabetes in a diabetes-high risk group.

When the suppressor of the present invention is used as an oral pharmaceutical composition, food or feed, the suppressor can be orally ingested by human or other mammals (e.g., dog, cat, mouse, rat, hamster, guinea pig, rabbit, swine, bovine, goat, horse, sheep, monkey, etc.) at a daily administration (ingestion) amount of, for example, 10⁴ to 10¹² phage particles (VLP), preferably 10⁵ to 10¹¹ VLP, once per day or in several divided doses. On the other hand, when the suppressor of the present invention is used as a parenteral pharmaceutical composition, the above-mentioned daily dose can be administered parenterally (e.g., rectal administration) once per day or in several divided doses.

When the suppressor of the present invention is used as a food, the food can be marketed with an indication that it is used for the elimination or reduction of diabetes-inducible bacteria belonging to Fuzimonas intestini in the intestine, and the prophylaxis and/or improvement of diabetes caused by the bacteria. As used herein, the “indication” refers to all acts of informing consumers of the above-mentioned use. Any indication that can remind or analogize the above-mentioned use falls under the “indication” in the present invention, regardless of the purpose of the indication, the content of the indication, the object, medium, and the like to be indicated. However, it is preferable to use an expression that allows consumers to directly recognize the above-mentioned use. Specific examples include the act of describing the above-mentioned use on a product or the packaging of a product relating to the food of the present invention, the acts of assigning, handing over, or exhibiting or importing for the purpose of assigning or handing over a product or the packaging of a product with description of the above-mentioned use, and the acts of displaying or distributing the above-mentioned use described in advertisements, price lists, or transaction documents relating to the product, or describing the above-mentioned use in the information with those contents and providing the information by an electromagnetic (Internet, etc.) method.

On the other hand, the indication is preferably approved by the government (for example, indication approved based on various systems established by the government, and performed in a manner based on such approval). In particular, indication on packaging, containers, catalogs, pamphlets, advertising materials at sales sites such as POP, and on other documents is preferred.

In addition, for example, indication of health food, functional food, enteral nutrition food, food for special dietary uses, food with nutrient function, quasi-drugs, and the like can be recited as examples. Also, other indications approved by the Ministry of Health, Labor and Welfare, such as foods for specified health use, and indication approved by a system similar thereto can be exemplified. Examples of the latter include indication of foods for specified health use, indication of foods for specified health use under certain conditions, indication of influence on the structure or function of the body, indication of disease risk reduction, and the like. To be specific, typical examples include indication of foods for specified health use (particularly, indication of health use) stipulated in the Ordinance for Enforcement of the Health Promotion Law (Ministry of Health, Labor and Welfare Ordinance No. 86, Apr. 30, 2003), and indications similar thereto.

In another embodiment of the present invention, the suppressor of the present invention can be used as a sterilizer and disinfectant for removing diabetes-inducing bacteria belonging to Fusimonas intestinalis that are mixed in a food ingested by humans and a feed ingested by other animals (including not only livestock feed but also pet food, etc.), as well as raw materials thereof (hereinafter, these are also collectively referred to as “foods, etc.”), or a sterilizer and disinfectant for removing the bacteria present in the equipment used for processing, cooking, and preserving foods, etc., and the bacteria present in the environment to which foods, etc. are exposed during the process thereof.

When the suppressor of the present invention is used as the above-mentioned sterilizer and disinfectant, it can be in the dosage form of, for example, a liquid or powder. The liquid may be provided in the form of spray, atomizer, or the like. Alternatively, it can also be provided in the form of a woven fabric, knitted fabric, non-woven fabric, or the like impregnated with the liquid.

When the suppressor of the present invention is used as the above-mentioned sterilizer and disinfectant, for example, it can be brought into contact with an object to be sterilized/disinfected by, for example, spraying, misting, immersion, wiping, or coating.

### II. Endolysin Preparation

The phage of the present invention is considered to take a lytic mode of destroying the cell wall of the host bacterium by the action of holin (encoded by ORF93 in strain LSP1), a transmembrane protein, and endolysin (encoded by ORF92 in strain LSP1), a degrading enzyme of cell wall peptidoglycan. Endolysin generally has a catalytic domain on the N-terminal side and a peptidoglycan substrate-binding domain on the C-terminal side, and the substrate-binding domain is specific for host cell wall structure. Thus, the protein alone can exhibit some degree of host specificity. In fact, when Uniprot blastx was run on the viral genome database using the nucleotide sequence of ORF92 of strain LSP1 as a query, the region from the N-terminal to about 200 nucleotides showed high amino acid homology with other bacteriophage-derived endolysin, but the region on the C-terminal side did not show high amino acid homology with known proteins. Therefore, the present inventors subcloned a fragment containing ORF92 from the genomic DNA of the strain LSP1, produced endolysin in Escherichia coli, added the recombinant enzyme to the medium of the strain AJ110941, and confirmed that bacteriolysis occurs rapidly. Accordingly, the present invention also provides a protein of any of the following (a) to (c) (hereinafter also to be referred to as “the endolysin of the present invention”):
(a) a protein consisting of the amino acid sequence represented by SEQ ID NO: 93;
(b) a protein comprising an amino acid sequence having 95% or more (e.g., 95, 96, 97, 98, 99% or more) identity with the amino acid sequence of (a), and capable of lysing a diabetes-inducible bacterium belonging to Fusimonas intestini;
(c) a protein comprising an amino acid sequence wherein 1 to several (e.g., 2, 3, 4, 5, 6) amino acids are substituted, deleted, inserted or added in the amino acid sequence of (a), and capable of lysing a diabetes-inducible bacterium belonging to Fusimonas intestini.

The amino acid sequence of the above-mentioned (a) corresponds to the amino acid sequence of endolysin encoded by ORF92 of the strain LSP1.

When the endolysin of the present invention contains the amino acid sequence of the above-mentioned (b) or (c), the region on the C-terminal side of the amino acid sequence (for example, an amino acid sequence of about 70 amino acid residues on the C-terminal side), which is regarded as the cell wall-binding domain, may be more conserved (for example, 98% or more, preferably 99% or more, identity with corresponding region in the amino acid sequence represented by SEQ ID NO: 93, or 1 or 2 amino acids are substituted, deleted, inserted or added in a corresponding region in the amino acid sequence represented by SEQ ID NO: 93), but is not limited thereto. The substitution of amino acids in the above-mentioned (c) is preferably substitution with similar amino acids.

The endolysin of the present invention can be produced, for example, by designing suitable primers to cover the endolysin-encoding region and cloning endolysin cDNA by PCR using the genomic DNA isolated from the phage of the present invention as a template, and digesting same with restriction enzymes when desired, or after adding an appropriate linker, inserting same into an expression vector suitable for expression in a host cell, introducing same into the host cell, culturing the host cell to allow for the expression of endolysin in the cell or medium.

In another embodiment, a DNA encoding the full length of the endolysin can also be constructed by connecting chemically synthesized partially overlapping oligo DNA short chains by using PCR method or Gibson Assembly method. The advantage of constructing full-length DNA by a combination of chemical synthesis and PCR method or Gibson Assembly method is that codons to be used can be designed over the entire length of CDS according to the host into which the DNA is introduced. When heterologous DNA is expressed, an increase in the expression level of protein can be expected by converting the DNA sequence into codons frequently used in the host organism. The data of codon usage frequency in the host to be used can be obtained from, for example, the genetic code usage frequency database (http://www.kazusa.or.jp/codon/index.html) published on the website of Kazusa DNA Research Institute, or documents showing codon usage frequency in each host can also be referred to. By referring to the obtained data and the DNA sequence to be introduced, codons with less usage frequency in the host from among the codons used in the DNA sequence, can be converted to codons that code for the same amino acid and have high usage frequency.

As vectors for producing recombinant endolysin, for example, Escherichia coli-derived plasmids (e.g., pBR322, pBR325, pUC12, pUC13, pCold); Bacillus subtilis-derived plasmids (e.g., pUB110, pTP5, pC194); yeast-derived plasmids (e.g., pSH19, pSH15); insect cell expression plasmids (e.g., pFast-Bac); animal cell expression plasmids (e.g., pA1-11, pXT1, pRc/CMV, pRc/RSV, pcDNAI/Neo); bacteriophages such as X phage and the like; insect virus vectors such as baculovirus (e.g., BmNPV, AcNPV); animal virus vectors such as retrovirus, vaccinia virus, adenovirus, and the like; and the like are used.

Any promoter may be used as long as it is suitable for the host used for gene expression. For example, when the host is Escherichia coli, trp promoter, lac promoter, recA promoter, λP_{L} promoter, lpp promoter, T7 promoter, cspA promoter and the like are preferred. When the host is Bacillus subtilis, SPO1 promoter, SPO2 promoter, penP promoter, and the like are preferred. When the host is a yeast, Gal1/10 promoter, PHO5 promoter, PGK promoter, GAP promoter, ADH promoter, and the like are preferred. When the host is an insect cell, polyhedrin promoter, P10 promoter, and the like are preferred. When the host is an animal cell, SRα, promoter, SV40 promoter, LTR promoter, CMV (cytomegalovirus) promoter, RSV (Rous sarcoma virus) promoter, MoMuLV (Moloney mouse leukemia virus) LTR, HSV-TK (simple herpes virus thymidine kinase) promoter and the like are preferred. When the host is a plant cell, CaMV 35S promoter, CaMV 19S promoter, NOS promoter, and the like are preferred.

As the host, for example, Escherichia coli, Bacillus subtilis, yeast, insect cell, insect, animal cell, and the like are used.

An expression vector can be introduced by a known method (e.g., lysozyme method, competent method, PEG method, CaCl₂ coprecipitation method, electroporation method, microinjection method, particle gun method, lipofection method, Agrobacterium method, and the like) according to the kind of the host.

Cells transfected with vectors can be cultured by a known method according to the kind of the host.

The endolysin produced by a conventional method can be recovered from the obtained culture of the host cell and purified using a protein separation technique known per se.

Alternatively, the endolysin of the present invention can also be synthesized in vitro using a cell-free transcription/translation system known per se using the above-mentioned endolysin-encoding DNA as a template.

The endolysin obtained as described above can lyse any diabetes-inducible bacterial strain belonging to Fusimonas intestini, and has bacteriolytic activity particularly against the strain AJ110941.

As described above, since the endolysin of the present invention can rapidly induce bacteriolysis by simply contacting the surface of a diabetes-inducible bacterium belonging to Fusimonas intestini, it can be used to suppress diabetes-inducible bacteria, like the phage of the present invention. Therefore, the present invention also provides a suppressor of a diabetes-inducible bacterium belonging to Fusimonas intestini, containing the endolysin of the present invention (hereinafter also to be referred to as “the suppressor (II) of the present invention”).

The endolysin of the present invention, like the phage of the present invention, can be formulated as it is or together with additives acceptable for pharmaceutical, or food or feed processing. Alternatively, the enzyme can be blended as a pharmaceutical additive or food or feed additive into a pharmaceutical composition or food or feed.

As the additives acceptable for pharmaceutical, or food or feed processing that are blended when the endolysin of the present invention is provided as a pharmaceutical composition, food, or feed, those described above with respect to the suppressor containing the phage of the present invention can be similarly recited as examples.

The amount of the endolysin of the present invention contained in the suppressor (II) of the present invention is, for example, 0.1 to 100 wt %.

The suppressor (II) of the present invention removes or reduces the diabetes-inducible bacteria belonging to Fusimonas intestini from the intestine of an animal. Therefore, it can be administered to (ingested by) patients (animal patients) suffering from diabetes caused by the bacteria, as a pharmaceutical composition, food or feed for treating and suppressing the progress in the patients (animal patients) and for preventing and delaying the onset in diabetes high-risk groups.

When the suppressor (II) of the present invention is used as an oral pharmaceutical composition, food or feed, the suppressor can be orally ingested by human or other mammals (body weight 60 kg) at a daily administration (ingestion) amount of, for example, 0.1 to 1000 mg, preferably 1 to 500 mg, once per day or in several divided doses. On the other hand, when the suppressor of the present invention is used as a parenteral pharmaceutical composition, the above-mentioned daily dose can be administered parenterally (e.g., rectal administration) once per day or in several divided doses.

In another embodiment of the present invention, the suppressor (II) of the present invention can be used as a sterilizer and disinfectant for removing diabetes-inducing bacteria belonging to Fusimonas intestinalis that are mixed in a food ingested by humans and a feed ingested by other animals (including not only livestock feed but also pet food, etc.), as well as raw materials thereof (hereinafter, these are also collectively referred to as “foods, etc.”), or a sterilizer and disinfectant for removing the bacteria present in the equipment used for processing, cooking, and preserving foods, etc., and the bacteria present in the environment to which foods, etc. are exposed during the process thereof.

As the dosage form and use form when the suppressor (II) of the present invention is used as the above-mentioned sterilizer and disinfectant, those described above with respect to the suppressor of the present invention can be preferably used in the same manner.

The endolysin of the present invention has bacteriolytic activity not only against diabetes-inducible bacteria belonging to Fusimonas intestini, but also against bacteria exhibiting resistance to one or more existing antibiotics. Therefore, the present invention also provides a suppressor of an antibiotic resistant bacterium, containing the endolysin of the present invention (hereinafter also to be referred to as “the suppressor (III) of the present invention”). The resistant bacterium that can be suppressed by the suppressor (III) of the present invention is not particularly limited and may be Gram-positive bacterium or Gram-negative bacterium. Examples thereof include methicillin-resistant Staphylococcus aureus (MRSA), multidrug-resistant Gram-positive cocci such as vancomycin-resistant enterococci (VRE), extended spectrum β-lactamase (ESBL)-producing bacteria, AmpC-type β-lactamase-producing bacteria, metallo-β-lactamase-producing bacteria, multidrug-resistant Pseudomonas aeruginosa (MDRP), and multidrug-resistant Gram-negative bacilli such as multidrug-resistant Acinetobacter (MDRA). In one preferred embodiment, the resistant bacteria include Gram-negative bacteria belonging to Acidovorax, which exhibit strong resistance to β-lactam antibiotics.

A biofilm is a collection of microorganisms wrapped in an extracellular matrix, and is closely related to the development of multidrug resistance in bacteria. The endolysin of the present invention has an inhibitory activity against biofilm formation. Thus, the suppressor (III) of the present invention is effective in suppressing resistant bacteria having biofilm-forming ability as one of their drug-resistance mechanisms.

The suppressor (III) of the present invention is formulated in the same manner as the suppressing agent (II) of the present invention, and can be administered to mammals infected with a bacterium resistant to one or more antibiotics by the same use method and in the same dosage, or can be applied (e.g., coated, sprayed, etc.) to an object (e.g., food, feed, or any other environmental substance) that is or may be contaminated with the bacterium. When the object to be suppressed is a Gram-negative bacterium, EDTA can be included in the formulation, or an EDTA solution can be simultaneously administered or added at the time of use, in order to improve outer membrane permeability and achieve permeation of endolysin to the cell wall existing inside the outer membrane. The amount of EDTA to be added is not particularly limited as long as it is sufficient for endolysin to permeate to the cell wall. For example, it can be appropriately selected such that the EDTA concentration at the site where the bacterium to be suppressed exists is 2 to 5 mM.

### III. Method for Detecting Diabetes-Inducible Bacterium

The present invention also provides a method of detecting a diabetes-inducible bacterium belonging to Fusimonas intestini in a sample, comprising contacting the phage of the present invention with the sample, and detecting infection of the bacteriophage with the diabetes-inducible bacterium (hereinafter also to be referred to as “the detection method of the present invention”).

The “sample” used in the detection method of the present invention is, for example, any sample collected from a living organism or nonliving object in which the presence of a diabetes-inducible bacterium belonging to Fusimonas intestini is suspected. Examples thereof include, but are not limited to, feces and intestinal contents collected from animals (e.g., mammals such as human, dog, cat, mouse, rat, hamster, guinea pig, rabbit, swine, bovine, goat, horse, sheep, monkey, and the like) suspected of containing the bacterium in their intestines.

In the detection method of the present invention, infection of the phage of the present invention with the diabetes-inducible bacterium can be detected by, for example, labeling the phage of the present invention with a labeling substance and detecting bacterial cells containing the labeling substance. For example, phage particles whose genomic DNA is labeled with ³²P can be obtained by culturing a host bacterium in a medium containing a ³²P-labeled phosphorus-containing substance and infecting the bacterium with the phage of the present invention to produce daughter phages. Alternatively, for example, using yeast or the like as a host, a marker protein gene such as a fluorescent protein (e.g., GFP, etc.) is inserted into an appropriate position (e.g., a position where it can be expressed as a fusion protein with a coat protein that constitutes the head or tail) on the phage genome by homologous recombination to prepare a recombinant phage that expresses the marker protein. The phage can then be contacted with a sample to infect the target diabetes-inducible bacterium.

Since the phage of the present invention is lytic, it is necessary to perform detection of bacterial cells that replicate and retain the phage in cells, before lysis begins. However, the bacteriolytic activity can be eliminated or attenuated by inactivating, for example, the gene of endolysin which is a lytic enzyme, or the gene of holin which is a transmembrane protein for efficiently delivering endolysin to the cell wall. Therefore, bacterial cells that have intracellularly accumulated a labeling substance can be easily detected by using phages in which the genes have been inactivated. Methods for inactivating lytic enzyme (including holin) gene include, for example, inserting a selection marker gene (e.g., drug resistance gene, auxotrophy-complementing gene, etc.) into the gene using homologous recombination, and selecting phage genomes in which the gene has been knocked out, by using drug resistance or proliferation in a nutrient-deficient medium as an index.

When a diabetes-inducible bacterium belonging to Fuzimonas intestini is detected from a sample by the detection method of the present invention, the bacterium can be exterminated from the animal, instrument, or other environment from which the sample was collected, by the above-mentioned suppressor of the present invention or the suppressor (II) of the present invention.

Other features of the invention will become apparent in the course of the following descriptions of exemplary embodiments which are given for illustration of the invention and are not intended to be limiting thereof.

### EXAMPLES

### Reference Example 1 Culture of Fusimonas Intestini AJ110941

This strain is an obligately anaerobic fermentative Gram-positive bacterium, and is a heterotrophic bacterium that grows well in a modified GAM broth medium (manufactured by Nissui Pharmaceutical Co., Ltd.), which is a medium for anaerobic bacteria (Sci Rep, 7.1: 18087 (2017), which is incorporated herein by reference in its entirety). The procedure for medium preparation and culture conditions are as follows.

First, 41.7 g/L of the modified GAM broth medium powder was added to ultrapure water sufficiently deaerated with N₂/CO₂ gas (80:20, v/v) and dissolved completely. The pH of the medium was adjusted to pH 7.8-8.0 using NaOH solution. The medium was dispensed into a glass vial, the liquid layer and air layer were sufficiently deaerated with N₂/CO₂ gas (80:20, v/v), and tightly sealed with a butyl rubber stopper and an aluminum cap (manufactured by Nichiden Rika Glass Co., Ltd.). The vial was sterilized with high pressure steam at 121° C. for 20 min and then stored at 4° C. until use.

A strain AJ110941 glycerol stock solution (5 mL) was inoculated into 45 mL of GAM medium and statically cultured at 37° C. The growth of this strain was directly confirmed by measurement of medium turbidity and microscopic observation. For passage culture, 1% of the preculture solution in which growth of the strain AJ110941 was confirmed was inoculated into a new GAM medium.

### Example 1 Purification and Isolation of LSP1 Phage

Phage particles were purified, concentrated, and separated from the culture supernatant of the F. intestini strain AJ110941 as follows.

First, the culture solution of the strain AJ110941 was centrifuged (8,000 rpm, 10 min) to remove bacterial cells. This culture supernatant was filtered through a filter with a pore size of 0.22 μm (manufactured by Merck Millipore). The phage fraction that passed through the filter was concentrated using a tangential flow filtration device (manufactured by Spectrum). The hollow fiber module used had a cutoff molecular weight of 300 kDa, and the membrane material used was modified polyethersulfone (manufactured by Spectrum), which has high filtration efficiency and high recovery rate. The phage solution after concentration was treated with DNase I (manufactured by Takara) and RNase A (manufactured by Nippon Gene) at 37° C. for 3 hr to remove DNA and RNA present outside the phage particles.

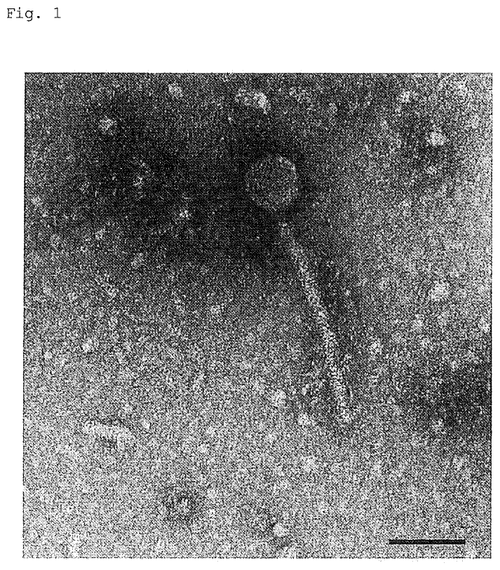

The concentrated phage solution was purified by density gradient centrifugation. An OptiSeal tube (manufactured by BECKMAN COULTER) was filled with an aqueous solution of Iodixanol (product name: OptiPrep, manufactured by Axis-Shield) with a density gradient of 30, 35, and 40% from the lower layer to the upper layer, and a phage concentrate was overlaid on the top layer. Centrifugation was performed under the conditions of 55,000 rpm, 24 hr, and 4° C. using an ultracentrifuge Optima MAX-TL and an MLA-50 rotor (manufactured by BECKMAN COULTER). After centrifugation, the phage fraction was recovered with a 21G needle (manufactured by TERUMO) and treated again with DNase I and RNase A at 37° C. for 3 hr. Furthermore, 40% Iodixanol aqueous solution, phage solution, and 30% Iodixanol aqueous solution were layered in this order from the lower layer of the OptiSeal tube, and centrifugation was performed again under the conditions of 55,000 rpm, 24 hr, and 4° C. A phage fraction was recovered with a 21G needle and stored at 4° C. until use. Morphological observation using electron microscope revealed that the LSP1 phage had a typical viral morphology belonging to the order Caudovirales with a head-tail structure (FIG. 1).

### Example 2 Purification and Sequence Analysis of LSP1 Phage Genome

Purification and sequence analysis of the phage genome were performed from the phage solution purified in Example 1.

First, the purified phage solution was treated again with DNase I and RNase A at 37° C. for 3 hr (DNA and RNA outside the phage particles were removed by 3 times in total of nuclease enzymatic treatments). Nucleic acid was extracted using Phage DNA Isolation Kit (manufactured by Norgen Biotech) according to a conventional method. A PCR experiment targeting the 16S rRNA gene confirmed that the genomic DNA of the host bacterium was not mixed. The PCR primers used therefor are as follows.
10F: 5′-GTTTGATCCTGGCTCA-3′ (SEQ ID NO: 100)
530F: 5′-GTGCCAGCMGCCGCGG-3′ (SEQ ID NO: 101)
907R: 5′-CCGTCAATTCMTTTRAGTTT-3′ (SEQ ID NO: 102)
1500R: 5′-TACCTTGTTACGACTT-3′ (SEQ ID NO: 103)

Next, in order to clarify the properties of the extracted phage genome, the LSP1 genome was treated with various nuclease enzymes (18 kinds of restriction enzymes (ApaI, BamHI, ClaI, EcoRI, EcoRV, DraI, HincII, HindIII, KpnI, NdeI, NotI, PstI, SacI, SalI, SmaI, SpnI, XbaI, XhoI, all manufactured by Takara), DNase I, RNase A, and P1 nuclease (manufactured by FUJIFIIM Wako Pure Chemical Corporation)), and the band patterns of agarose gel electrophoresis were confirmed. The genome was not degraded at all by restriction enzymes and RNase A, whereas degraded by DNase I and P1 nuclease. Therefore, this phage genome was determined to be single-stranded DNA. Amplification and purification of single-stranded DNA were performed with reference to previous study (Proc. Natl. Acad. Sci. USA, 102. 36: 12891-12896). The polymerase enzyme used was 3′-5′ exo-Klenow DNA polymerase (manufactured by New England Biolabs), and the primer sequences are as follows.
FR26RV-N: 5′-GCCGGAGCTCTGCAGATATCNNNNNN-3′ (SEQ ID NO: 104)
FR20RV: 5′-GCCGGAGCTCTGCAGATATC-3′ (SEQ ID NO: 105)

The PCR product was purified using a QIAquick gel extraction kit (manufactured by Qiagen) and cloned into pT7Blue T-vector (manufactured by Novagen). Escherichia coli DH5α competent cells (manufactured by GMbiolab) were transformed with this plasmid. E. coli transformants retaining the insert DNA were selected by blue-white determination and colony PCR method, and plasmid DNA was extracted using QIAprep Spin Miniprep Kit (manufactured by QIAGEN).

The insert DNA was sequenced using the extracted plasmid DNA as a template and using Applied Biosystems 3130/3130xl Genetic Analyzers (manufactured by Applied Biosystems). The sequences of the primers used are as follows.
M13 primer M4: 5′-GTTTTCCCAGTCACGAC-3′ (SEQ ID NO: 106)
M13 primer RV: 5′-CAGGAAACAGCTATGAC-3′ (SEQ ID NO: 107)

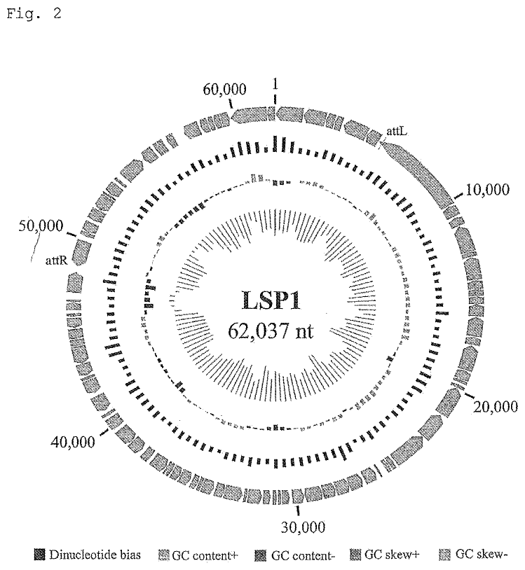

Using the decoded sequence information, homology search was performed by NCBI BLAST (https://blast.ncbi.nlm.nih.gov/Blast.cgi) (Non-redundant protein sequence was referenced for the database). New PCR primers were prepared from the obtained sequence information, and the entire phage genome was decoded by the primer walking method. The nucleotide sequence was assembled using GeneStudio software. As a result, the genome of this phage was found to be cyclic single-stranded DNA with a size of 62,037 nt and a G+C content of 38.83% (FIG. 2). Prediction and annotation of Open Reading Frames were performed using the NCBI ORF Finder (http://www.ncbi.nlm.nih.gov/projects/gorf/), PHAge Search Tool (PHAST, http://phast.wishartlab.com/), and PHAge Search Tool Enhanced Release (PHASTER, www.phaster.ca). As a result, 98 ORFs were found from the phage genome (Tables 1-1 and 1-2). Although most of these ORFs have unknown functions, genes characteristic of phage were annotated, such as genes involved in phage morphogenesis (head-tail structure, filaments), DNA packaging and replication (Terminase and Portal proteins), gene recombination (Integrase and Transposase), and lysis of host bacterium (endolysin).

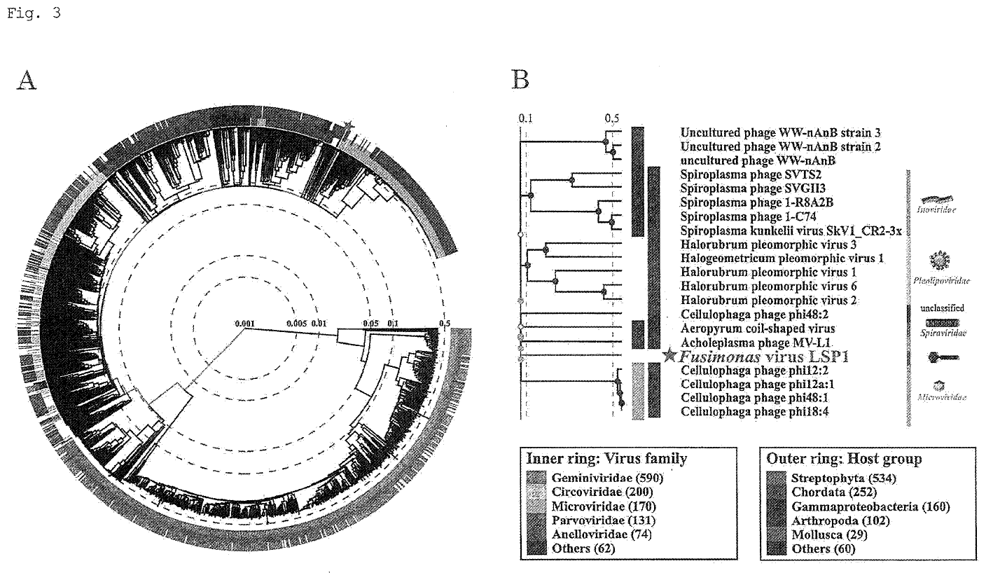

It is known that the genome size of single-stranded DNA viruses is smaller than that of double-stranded DNA viruses, and is generally around 5-10 knt. The genome size of the largest single-stranded DNA virus discovered so far was 24,893 nt (57 ORFs) of Aeropyrum coil-shaped virus, a hyperthermogenic archaeal virus (Proc. Natl. Acad. Sci. USA, 109. 33: 13386-13391 (2012)). Therefore, the phage is currently the single-stranded DNA virus with the largest genome. Furthermore, it has been reported that viruses belonging to the order Caudovirales with a head-tail structure all have a linear double-stranded DNA as their genomes. Even though the phage had a head-tail structure, its genome was a cyclic single-stranded DNA. Since virus phylogeny is based on the morphology and genome structure of virus particles (Viruses 9. 4: 70 (2017)), it was suggested that this phage may be a taxonomically extremely novel virus (Table 2). In fact, when an analysis was performed using the phylogenetic classification server VipTree based on the viral genome (Bioinformatics, 33.15: 2379-2380 (2017)), the phage was certainly phylogenetically completely different from known single-stranded DNA viruses, and was supported to be novel even at the genome level (FIG. 3).

From the above results, it was confirmed that the phage was an extremely novel phage constituting at least a novel Order, and the phage was named strain LSP1.

TABLE 2
comparison of LSP1 phage and known viruses

| Order | New Order | Candovirales | | | | | | |
|---|---|---|---|---|---|---|---|---|
| Family | New Family | Myoviridae | Podoviridae | Siphoviridae | Inoviridae | Microviridae | Pleolipoviridae | Spiraviridae |
| Host | F. intestini | Bacteria and Archaea | | | Bacteria | Bacteria | Archaea | A. pernix |
| Morphology | Head-tailed | Head-tailed | | | Filamentous | Icosahedral | Pleomorphic | Coli-shaped |
| Genome type | Circular | Linear | | | Circular | Circular | Linear/Circular | Circular |
| | ssDNA | dsDNA | | | ssDNA | ssDNA | ss/dsDNA | ssDNA |
| Genome size | 62,037 | 33-244 | 16-70 | 22-121 | 4.5-12.4 | 4.4-6.1 | 7.0-16.0 | 24,893 |
| (kbp/km) | | | | | | | | |
| No. of ORFs | 98 | 40-415 | 20-65 | 36-88 | 4-16 | 8-11 | 9-35 | 57 |
| Cell lysis | Holin and | Holin and peptidoglycan hydrolases | | | No cell lysis | Peptidoglycan | No cell lysis | unknown |
| | peptidoglycan | | | | (growth delay) | synthesis | (growth delay) | |
| | hydrolases | | | | | inhibitor | | |
| Infection | Lytic | Lytic/Lysogenic | | | Chronic | Lytic | Lysogenic | unknown |
| mode | | | | | infection | | | |

### Example 3 Growth Suppression Test of Strain AJ110941 by Addition of LSP1 Phage

In order to verify whether the LSP1 phage purified and isolated in Example 1 infects the diabetes-inducible bacterium F. intestini strain AJ110941 and causes bacteriolysis, F. intestini was infected with purified LSP1 in vitro, and turbidity and cell morphology were analyzed.

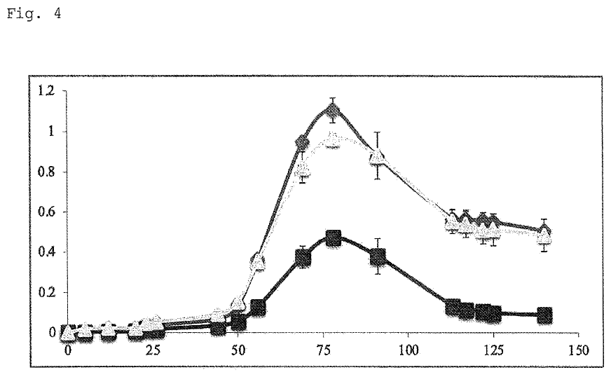

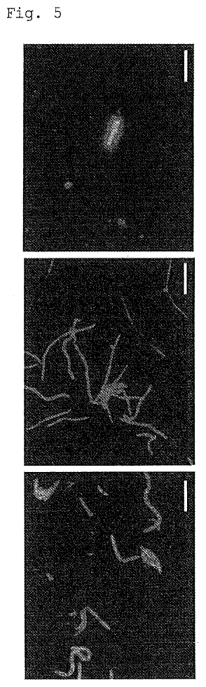

The test was divided into three groups (medium only addition group, inactivated LSP1 addition group, LSP1 addition group), and each was conducted in triplicate. LSP1 phage was inactivated by high-pressure vapor sterilization (121° C., 20 min). As a result of the infection experiment, it was found that the growth of F. intestini was markedly suppressed in the LSP1 addition group as compared with the medium-only addition group and the inactivated LSP1 addition group (FIG. 4). In addition, microscopic observation showed that F. intestini was lysed and the cell morphology was destroyed in the LSP1 addition group (FIG. 5). From the above results, the LSP1 phage was determined to be a lytic phage.

Next, in order to investigate the host specificity of the LSP1 phage, type strains (Anaerostipes caccae JCM13470^{T}, Blautia hydrogenotrophica DSM10507^{T}, Clostridium amygdalinum DSM12857^{T}, Clostridium citroniae RAM16102^{T}, Desulfotamaculum guttoideum DSM 4024^{T}, Eisenbergiella tayi B086562^{T}, Eubacterium fissicatena DSM3598^{T}, Murimonas intestini SRB-530-5-HT^{T}, Ruminococcus gauvreauii 14987^{T}) belonging to the Lachnospiraceae family, similar to F. intestini, were purchased from culture collection institutes, and infection experiments were conducted in the same manner as above. Since LSP1 did not show any infection or bacteriolytic activity against these type strains, it was suggested that this phage may be a phage that specifically infects F. intestini.

### Example 4 Storage Stability of LSP1 Phage

It is generally known that phage solutions can be stably stored at 4° C., and the concentrated and purified LSP1 solution was also stored at 4° C. until use. When the infectivity of F. intestini strain AJ110941 was confirmed using the LSP1 solution stored at 4° C. for one month, the bacteriolytic activity was found. Thus, it was shown that LSP1 can be stably stored at 4° C. for at least one month.

### Example 5 Lysis of Strain AJ110941 by LSP1 Phage-Derived Endolysin

A recombinant plasmid was produced by inserting the PCR-amplified ORF92 (lysF) gene region into the NdeI/EcoRI site of the high expression vector pColdII (manufactured by TaKaRa) (see below for primer sequences).

Escherichia coli DH5α competent cells were transformed with this plasmid by a heat shock method. Escherichia coli transformants were selected by colony PCR, and plasmid DNA was purified from the culture solution of the positive clones by using QIAprep Spin Miniprep Kit (manufactured by QIAGEN).

Forward:
(SEQ ID NO: 108)
5′-GGAATTCCATATGAGATTTACCAATAGTCCGCTG-3′
(NdeI site)
Reverse:
(SEQ ID NO: 109)
5′-CCGGAATTCCACTTGTTTAAATGTCTGACGTGTA-3′
(EcoRI site)

Escherichia coli BL21 (DE3) pLysS competent cells (manufactured by BioDynamics Laboratory) were transformed with the obtained plasmid. The obtained transformant colonies were cultured by liquid-culture at 37° C. until the OD₆₀₀ value reached around 0.4-0.6. The culture solution was allowed to stand at 15° C. for 30 min, isopropyl β-D-1-thiogalactopyranoside (IPTG) was added at a final concentration of 100 μM, and shaking culture was performed at 15° C. for 24 hr. After collecting the bacterial cells, the cells were ultrasonically disrupted, and the soluble fraction was purified by nickel affinity chromatography to obtain a recombinant endolysin (LysF) solution.

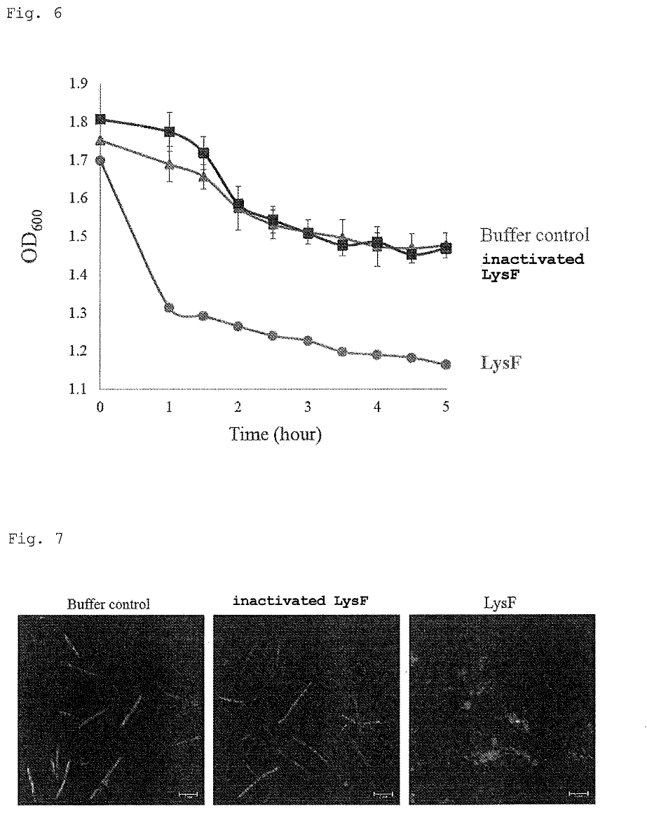

The purified LysF enzyme was added to the culture solution of F. intestini and the turbidity (OD₆₀₀ value) was measured over time. As a result, a significant decrease in turbidity was observed in the LysF-added system as compared with the control to which Buffer was added (FIG. 6). Microscopic observation of the cell morphology of F. intestini after addition of LysF revealed marked bacteriolysis (FIG. 7). Therefrom it was suggested that the experimental result of turbidity reduction shown in FIG. 6 was caused by the lysis of F. intestini by the addition of LysF. On the other hand, the inactivated LysF-added system exhibited turbidity similar to that of the control, and remarkable bacteriolysis was not observed (FIGS. 6 and 7). The above results have clarified that LysF derived from LSP1 phage is a novel endolysin with bacteriolytic activity.

### Example 6 Measurement of Enzyme Activity of Recombinant LysF Against Multidrug-Resistant Bacteria

In recent years, the emergence of multidrug-resistant bacteria that are highly resistant to multiple antibacterial drugs has become a problem around the world, especially in the medical field. Phages and phage-derived endolysins are attracting attention as countermeasures against multidrug-resistant bacteria in the present age when the development of new antibacterial agents tends to be stalled. The present inventors have so far isolated several multidrug-resistant bacteria with extremely high resistance to β-lactam antibiotics from the wastewater processing system of an antibiotic production plant (Kusada, H., Tamaki, H., Kamagata, Y., Hanada, S., & Kimura, N. (2017). A novel quorum-quenching N-acylhomoserine lactone acylase from Acidovorax sp. strain MR-S7 mediates antibiotic resistance. Appl. Environ. Microbiol., 83(13), e00080-17; Kusada, H., Zhang, Y., Tamaki, H., Kimura, N., & Kamagata, Y. (2019). Novel N-acyl homoserine lactone-degrading bacteria isolated from penicillin-contaminated environments and their quorum-quenching activities. Front microbiol, 10, 455.). In this Example, the bacteriolytic activity of the LysF enzyme against 3 strains of the genus Acidovorax (MR-S7, M2, M6) having multidrug resistance was evaluated.

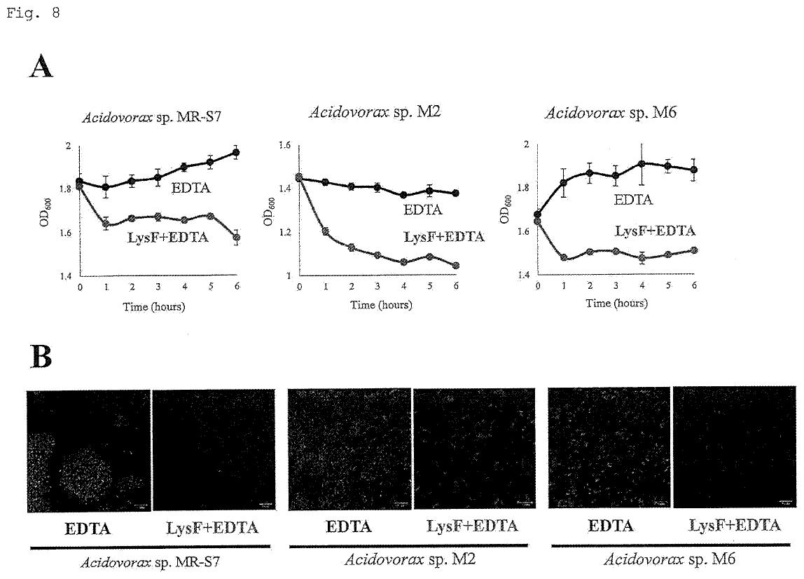

The enzyme activity of LysF was evaluated by monitoring over time the decrease in turbidity of resistant bacteria. Specifically, 3 strains of resistant bacteria were cultured in LB medium until the logarithmic growth phase, and the bacterial cells recovered from the culture solution by centrifugation were resuspended in a buffer. LysF solution and EDTA (final concentration 2.5 mM) were added to this suspension and the mixture was incubated at 30° C. (EDTA is known to improve the outer membrane permeability of Gram-negative bacteria and was added to cause penetration of LysF to the cell wall present inside the outer membrane). The turbidity (OD₆₀₀ value) of these reaction mixtures was measured every hour. As a result, a significant decrease in turbidity was observed in the LysF-added system as compared with the control to which EDTA alone was added (FIG. 8A). In addition, as a result of microscopic observation of various samples 6 hr after the reaction, lysis of cells or a decrease in the number of bacterial cells was confirmed (FIG. 8B). The above results have clarified that LysF derived from LSP1 phage is a novel endolysin showing bacteriolytic activity even for multidrug-resistant bacteria.

### Example 7 Verification Experiment of Inhibitory Effect of Recombinant LysF on Biofilm Formation in Multidrug-Resistant Bacteria

A biofilm is a collection of microorganisms wrapped in an extracellular matrix, and is closely related to the development of multidrug resistance in bacteria. It has been found that multidrug-resistant bacterium Acidovorax sp. strain MR-S7 forms a biofilm under conditions with the addition of acylated homoserine lactone which is a substance for communication between bacteria, and improves resistance to various antibiotics (neomycin, gentamicin, tetracycline, chloramphenicol) (Kusada, H., Hanada, S., Kamagata, Y., & Kimura, N. (2014). The effects of N-acylhomoserine lactones, β-lactam antibiotics and adenosine on biofilm formation in the multi-β-lactam antibiotic-resistant bacterium Acidovorax sp. strain MR-S7. J Biosci Bioeng, 118(1), 14-19.). In this Example, the biofilm formation inhibitory effect of LysF against multidrug-resistant bacteria that form biofilms was verified.

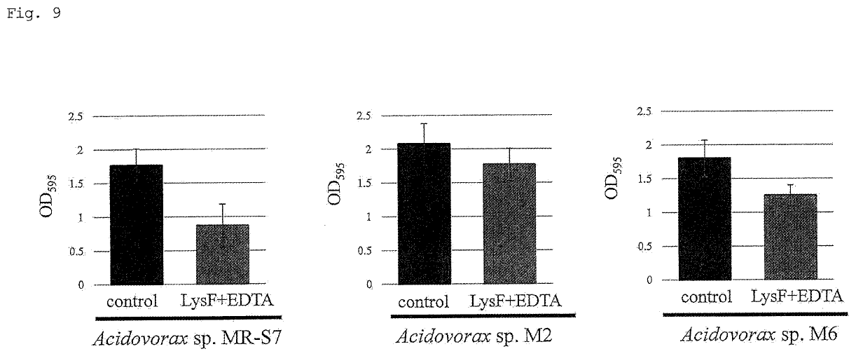

The culture solution of 3 strains of Acidovorax bacteria (MR-S7, M2, M6) precultured in LB medium was inoculated at 1% in new LB medium (using a 96 well plate). N-(3-oxo-octanoyl)-L-homoserine lactone (OC8-HSL) was added to each well to a final concentration of 5 μM, and the cells were statically cultured at 30° C. for 5 days. The shape of the biofilm formed varies depending on the strain, and the strain MR-S7 formed a biofilm in the form of a ring, the strain M2 formed a biofilm on the bottom of the plate, and the strain M6 formed a biofilm on the air-liquid interface. LysF and EDTA (final concentration: 10 mM) were added to these biofilms and statically cultured at 30° C. for 4 hr. Biofilm was quantified according to a conventional method using crystal violet. As a result, it was clarified that the amount of biofilm formation (OD₅₉₅ value) decreased in the LysF-added group as compared with the control group (FIG. 9). From the above, it was found that LysF derived from LSP1 phage also has the effect of inhibiting biofilm formation of multidrug-resistant bacteria.

### INDUSTRIAL APPLICABILITY

The phage of the present invention can specifically infect and lyse diabetes-inducible bacteria belonging to Fusimonas intestini. Therefore, it can selectively remove or reduce diabetes-inducible bacterium from an animal having the bacterium in its intestine, without affecting the survival of enteric bacteria useful for the animal. Also, the endolysin of the present invention can also rapidly lyse diabetes-inducible bacteria belonging to Fusimonas intestini. Furthermore, the endolysin of the present invention also has bacteriolytic activity against drug-resistant bacteria including multidrug-resistant bacteria. Endolysin preparations have further advantages since it is extremely difficult for bacteria to be resistant to endolysin.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A bacteriophage capable of infecting and lysing a diabetes-inducible bacterium belonging to Fusimonas intestini, comprising a cyclic single-stranded DNA, which consists of the nucleotide sequence represented by SEQ ID NO: 1 or a nucleotide sequence having 90% or more identity thereto, as its genome.

2. The bacteriophage according to claim 1, comprising at least a nucleotide sequence encoding any of the amino acid sequences in the following (a) to (c):
(a) the amino acid sequence represented by SEQ ID NO: 99;
(b) an amino acid sequence having 95% or more identity with the amino acid sequence of (a);
(c) an amino acid sequence wherein 1 to several amino acids are substituted, deleted, inserted or added in the amino acid sequence of (a).

3. The bacteriophage according to claim 2, further comprising 97 kinds of nucleotide sequences encoding any of the 97 kinds of amino acid sequences, respectively, in the following (d) to (f):
(d) 97 kinds of amino acid sequences represented by SEQ ID NOs: n, wherein n is an integer of 2-98;
(e) 97 kinds of amino acid sequences each having 95% or more identity with the respective amino acid sequences shown in (d);
(f) 97 kinds of amino acid sequences shown in (d), wherein 1 to several amino acids are substituted, deleted, inserted or added in one or more sequences thereof.

4. The bacteriophage according to claim 1, wherein the diabetes-inducible bacterium is Fusimonas intestini strain AJ110941 (FERM BP-11443).

5. The bacteriophage according to claim 1, comprising a cyclic single-stranded DNA, which consists of the nucleotide sequence represented by SEQ ID NO: 1, as its genome.

6. A protein of any of the following (a) to (c):
(a) a protein consisting of the amino acid sequence represented by SEQ ID NO: 93;
(b) a protein comprising an amino acid sequence having 95% or more identity with the amino acid sequence of (a), and capable of lysing a diabetes-inducible bacterium belonging to Fusimonas intestini;
(c) a protein comprising an amino acid sequence wherein 1 to several amino acids are substituted, deleted, inserted or added in the amino acid sequence of (a), and capable of lysing a diabetes-inducible bacterium belonging to Fusimonas intestini.

7. The protein according to claim 6, wherein the diabetes-inducible bacterium is Fusimonas intestini strain AJ110941 (FERM BP-11443).

8. A nucleic acid encoding the protein according to claim 6.

9. A method of producing the protein according to claim 6, comprising synthesizing the protein in an expression system comprising the nucleic acid encoding the protein in a form capable of expressing the same.

10. An agent for suppressing a diabetes-inducible bacterium belonging to Fusimonas intestini, comprising the bacteriophage according to claim 1.

11. The agent according to claim 10 for removing or reducing the diabetes-inducible bacterium from the intestine of an animal.

12. The agent according to claim 11 for the prophylaxis or treatment of diabetes in the animal.

13. A method of detecting a diabetes-inducible bacterium belonging to Fusimonas intestini in a sample, comprising contacting the bacteriophage according to claim 1 with the sample, and detecting infection of the bacteriophage with the diabetes-inducible bacterium.

14. The method according to claim 13, wherein a lytic enzyme gene of the bacteriophage is inactivated.

15. An agent for suppressing a bacterium resistant to one or more antibiotics, comprising the protein according to claim 6.

16. The agent according to claim 15, wherein the bacterium can form a biofilm.

17. An agent for suppressing a diabetes-inducible bacterium belonging to Fusimonas intestini, comprising the protein according to claim 6.
